(19) United States
(12) Patent Application Publication
(10) Pub. No.: US 2023/0210981 A1
(43) Pub. Date: Jul. 6, 2023

(54) CORONAVIRUS DISEASE 2019 (COVID-19) COMBINATION VACCINE

(71) Applicant: The Wistar Institute of Anatomy and Biology, Philadelphia, PA (US)

(72) Inventors: David Weiner, Merion, PA (US); Ami Patel, Philadelphia, PA (US)

(21) Appl. No.: 18/001,116

(22) PCT Filed: Jun. 9, 2021
(86) PCT No.: PCT/US21/36578

§ 371 Date: Dec. 8, 2022

Related U.S. Application Data
(60) Provisional application No. 63/036,696, filed on Jun. 9, 2020.

(51) Int. Cl. A61K 39/215; A61P 31/14

(52) U.S. Cl. CPC A61K 39/215; A61P 31/14; A61K 2039/53

(57) ABSTRACT

Disclosed herein is a vaccine comprising a Coronavirus disease 2019 (COVID-19) antigen in a combination with an immunoglobulin from post-exposure treatment. The antigen can be a consensus antigen. The consensus antigen can be a consensus spike antigen. Also disclosed herein is a method of treating a subject in need thereof, by administering the vaccine to the subject.

### CROSS REFERENCE TO RELATED APPLICATIONS

This application claims priority to U.S. Provisional Application No. 63/036,696, filed Jun. 9, 2020 which is hereby incorporated by reference herein in its entirety.

### TECHNICAL FIELD

The present invention relates to a combination vaccine for Coronavirus disease 2019 (COVID-19) and a method of administering the vaccine.

### BACKGROUND

Coronaviruses (CoV) are a family of viruses that are common worldwide and cause a range of illnesses in humans from the common cold to severe acute respiratory syndrome (SARS). Coronaviruses can also cause a number of diseases in animals. Human coronaviruses 229E, OC43, NL63, and HKU1 are endemic in the human population.

Coronavirus disease 2019 (COVID-19), known previously as 2019-nCoV pneumonia or disease, has rapidly emerged as a global public health crisis, joining severe acute respiratory syndrome (SARS) and Middle East respiratory syndrome (MERS) in a growing number of coronavirus-associated illnesses which have jumped from animals to people. There are at least seven identified coronaviruses that infect humans. In December 2019 the city of Wuhan in China became the epicenter for an outbreak of the novel coronavirus, severe acute respiratory syndrome coronavirus 2 (SARS-CoV-2). SARS-CoV-2 was isolated and sequenced from human airway epithelial cells from infected patients (Zhu et al., 2020 N Engl J Med, 382:727-733; Wu et al., 2020, Nature, 579:265-269). Disease symptoms can range from mild flu-like to severe cases with life-threatening pneumonia (Huang et al., 2020, Lancet, 395:497-506). The global situation is dynamically evolving, and on Jan. 30, 2020 the World Health Organization declared COVID-19 as a public health emergency of international concern (PHEIC) and on Mar. 11, 2020 it was declared a global pandemic. As of Apr. 1, 2020 there are 932,605 people infected and 46,809 deaths (gisaid.org/epiflu-applications/global-cases-covid-19). Infections have spread to multiple continents. Human-to-human transmission has been observed in multiple countries, and a shortage of disposal personal protective equipment, and prolonged survival times of coronaviruses on inanimate surfaces (Hulkower et al., 2011, Am J Infect Control 39, 401-407), have compounded this already delicate situation and heightened the risk of nosocomial infections. Advanced research activities must be pursued in parallel to push forward protective modalities in an effort to protect billions of vulnerable individuals worldwide. Currently, no licensed preventative vaccine or specific anti-viral therapy is available for COVID-19.

Accordingly, a need remains in the art for the development of a safe and effective vaccine that is applicable to COVID-19, thereby providing protection against and promoting survival of COVID-19 and/or SARS-CoV-2 infection.

### BRIEF SUMMARY OF THE INVENTION

In one embodiment, the invention relates to an immunogenic composition comprising intravenous immunoglobulin (IVIG) generated from one or more host subject vaccinated against Coronavirus disease 2019 (COVID-19) or diagnosed as having COVID-19 and subsequently cured of COVID-19.

In one embodiment, the host subject was vaccinated with a nucleic acid molecule comprising a nucleotide sequence selected from the group consisting of: a nucleotide sequence encoding a peptide comprising an amino acid sequence having at least about 90% identity over an entire length of the amino acid sequence set forth in SEQ ID NO:2; a nucleotide sequence encoding a peptide comprising an amino acid sequence having at least about 90% identity over an entire length of the amino acid sequence set forth in SEQ ID NO:4; a nucleotide sequence encoding a peptide comprising an amino acid sequence having at least about 90% identity over an entire length of the amino acid sequence set forth in SEQ ID NO:6; and a nucleotide sequence encoding a peptide comprising an amino acid sequence having at least about 90% identity over an entire length of the amino acid sequence set forth in SEQ ID NO:8.

In one embodiment, the host subject was vaccinated with a nucleic acid molecule comprising a nucleotide sequence encoding the amino acid sequence selected from the group consisting of SEQ ID NO:2, SEQ ID NO:4, SEQ ID NO:6 and SEQ ID NO:8.

In one embodiment, the host subject was vaccinated with a nucleic acid molecule comprising a nucleotide sequence selected from the group consisting of: the nucleotide sequence having at least about 90% identity over an entire length of the nucleic acid sequence set forth in SEQ ID NO:1; the nucleotide sequence having at least about 90% identity over an entire length of the nucleic acid sequence set forth in SEQ ID NO:3; the nucleotide sequence having at least about 90% identity over an entire length of the nucleic acid sequence set forth in SEQ ID NO:5; and the nucleotide sequence having at least about 90% identity over an entire length of the nucleic acid sequence set forth in SEQ ID NO:7.

In one embodiment, the nucleic acid molecule comprises the nucleic acid sequence selected from the group consisting of SEQ ID NO:1, SEQ ID NO:3, SEQ ID NO:5 and SEQ ID NO:7.

In one embodiment, the immunoglobulin is extracted from a biological sample.

In one embodiment, the biological sample is a plasma, blood, or a combination thereof.

In one embodiment, the immunogenic composition further comprises a nucleic acid molecule comprising a nucleotide sequence selected from the group consisting of: a nucleotide sequence encoding a peptide comprising an amino acid sequence having at least about 90% identity over an entire length of the amino acid sequence set forth in SEQ ID NO:2; a nucleotide sequence encoding a peptide comprising an amino acid sequence having at least about 90% identity over an entire length of the amino acid sequence set forth in SEQ ID NO:4; a nucleotide sequence encoding a peptide comprising an amino acid sequence having at least about 90% identity over an entire length of the amino acid sequence set forth in SEQ ID NO:6; and a nucleotide sequence encoding a peptide comprising an amino acid sequence having at least about 90% identity over an entire length of the amino acid sequence set forth in SEQ ID NO:8.

In one embodiment, the nucleic acid molecule comprises a nucleotide sequence encoding the amino acid sequence selected from the group consisting of SEQ ID NO:2, SEQ ID NO:4, SEQ ID NO:6 and SEQ ID NO:8.

In one embodiment, the nucleic acid molecule comprises a nucleotide sequence selected from the group consisting of: the nucleotide sequence having at least about 90% identity over an entire length of the nucleic acid sequence set forth in SEQ ID NO:1; the nucleotide sequence having at least about 90% identity over an entire length of the nucleic acid sequence set forth in SEQ ID NO:3; the nucleotide sequence having at least about 90% identity over an entire length of the nucleic acid sequence set forth in SEQ ID NO:5; and the nucleotide sequence having at least about 90% identity over an entire length of the nucleic acid sequence set forth in SEQ ID NO:7.

In one embodiment, the nucleic acid molecule comprises the nucleic acid sequence selected from the group consisting of SEQ ID NO:1, SEQ ID NO:3, SEQ ID NO:5 and SEQ ID NO:7.

In one embodiment, the nucleic acid molecule comprises an expression vector.

In one embodiment, the nucleic acid molecule is incorporated into a viral particle.

In one embodiment, the immunogenic composition further comprises a pharmaceutically acceptable excipient.

In one embodiment, the immunogenic composition further comprises an adjuvant.

In one embodiment, the invention provides a method of generating an immunogenic composition comprising intravenous immunoglobulin (IVIG) from one or more host subject, the method comprising: a) administering a nucleic acid molecule encoding a SARS-CoV-2 antigen to the host subject; and b) isolating a biological sample comprising one or more immunoglobulin molecule from the host subject.

In one embodiment, the subject has been diagnosed as having SARS-CoV-2 infection or COVID-19, or has is considered cured of SARS-CoV-2 infection or COVID-19.

In one embodiment, the nucleic acid molecule comprises a nucleotide sequence selected from the group consisting of: a nucleotide sequence encoding a peptide comprising an amino acid sequence having at least about 90% identity over an entire length of the amino acid sequence set forth in SEQ ID NO:2; a nucleotide sequence encoding a peptide comprising an amino acid sequence having at least about 90% identity over an entire length of the amino acid sequence set forth in SEQ ID NO:4; a nucleotide sequence encoding a peptide comprising an amino acid sequence having at least about 90% identity over an entire length of the amino acid sequence set forth in SEQ ID NO:6; and a nucleotide sequence encoding a peptide comprising an amino acid sequence having at least about 90% identity over an entire length of the amino acid sequence set forth in SEQ ID NO:8.

In one embodiment, the nucleic acid molecule comprises a nucleotide sequence encoding the amino acid sequence selected from the group consisting of SEQ ID NO:2, SEQ ID NO:4, SEQ ID NO:6 and SEQ ID NO:8.

In one embodiment, the nucleic acid molecule comprises a nucleotide sequence selected from the group consisting of: the nucleotide sequence having at least about 90% identity over an entire length of the nucleic acid sequence set forth in SEQ ID NO:1; the nucleotide sequence having at least about 90% identity over an entire length of the nucleic acid sequence set forth in SEQ ID NO:3; the nucleotide sequence having at least about 90% identity over an entire length of the nucleic acid sequence set forth in SEQ ID NO:5; and the nucleotide sequence having at least about 90% identity over an entire length of the nucleic acid sequence set forth in SEQ ID NO:7.

In one embodiment, the nucleic acid molecule comprises the nucleic acid sequence selected from the group consisting of SEQ ID NO:1, SEQ ID NO:3, SEQ ID NO:5 and SEQ ID NO:7.

In one embodiment, the biological sample is a plasma, blood, or a combination thereof.

In one embodiment, the invention relates to a method of inducing an immune response against SARS-CoV-2 in a subject in need thereof, the method comprising administering an immunogenic composition comprising intravenous immunoglobulin (IVIG) generated from one or more host subject vaccinated against Coronavirus disease 2019 (COVID-19), or diagnosed as having COVID-19 and subsequently cured of COVID-19, to the subject.

In one embodiment the method further comprises administering a nucleic acid molecule encoding a SARS-CoV-2 antigen to the subject.

In one embodiment the nucleic acid molecule comprises a nucleotide sequence selected from the group consisting of: a nucleotide sequence encoding a peptide comprising an amino acid sequence having at least about 90% identity over an entire length of the amino acid sequence set forth in SEQ ID NO:2; a nucleotide sequence encoding a peptide comprising an amino acid sequence having at least about 90% identity over an entire length of the amino acid sequence set forth in SEQ ID NO:4; a nucleotide sequence encoding a peptide comprising an amino acid sequence having at least about 90% identity over an entire length of the amino acid sequence set forth in SEQ ID NO:6; and a nucleotide sequence encoding a peptide comprising an amino acid sequence having at least about 90% identity over an entire length of the amino acid sequence set forth in SEQ ID NO:8.

In one embodiment the nucleic acid molecule comprises a nucleotide sequence encoding the amino acid sequence selected from the group consisting of SEQ ID NO:2, SEQ ID NO:4, SEQ ID NO:6 and SEQ ID NO:8.

In one embodiment the nucleic acid molecule comprises a nucleotide sequence selected from the group consisting of: the nucleotide sequence having at least about 90% identity over an entire length of the nucleic acid sequence set forth in SEQ ID NO:1; the nucleotide sequence having at least about 90% identity over an entire length of the nucleic acid sequence set forth in SEQ ID NO:3; the nucleotide sequence having at least about 90% identity over an entire length of the nucleic acid sequence set forth in SEQ ID NO:5; and the nucleotide sequence having at least about 90% identity over an entire length of the nucleic acid sequence set forth in SEQ ID NO:7.

In one embodiment the nucleic acid molecule comprises the nucleic acid sequence selected from the group consisting of SEQ ID NO:1, SEQ ID NO:3, SEQ ID NO:5 and SEQ ID NO:7.

In one embodiment, administering includes at least one of electroporation and injection.

In one embodiment, the invention provides a method of protecting a subject in need thereof from infection with SARS-CoV-2 or COVID-19, the method comprising administering an immunogenic composition comprising intravenous immunoglobulin (IVIG) generated from one or more host subject vaccinated against Coronavirus disease 2019 (COVID-19), or diagnosed as having COVID-19 and subsequently cured of COVID-19, to the subject.

In one embodiment, the method further comprises administering a nucleic acid molecule encoding a SARS-CoV-2 antigen to the subject.

In one embodiment, the nucleic acid molecule comprises a nucleotide sequence selected from the group consisting of: a nucleotide sequence encoding a peptide comprising an amino acid sequence having at least about 90% identity over an entire length of the amino acid sequence set forth in SEQ ID NO:2; a nucleotide sequence encoding a peptide comprising an amino acid sequence having at least about 90% identity over an entire length of the amino acid sequence set forth in SEQ ID NO:4; a nucleotide sequence encoding a peptide comprising an amino acid sequence having at least about 90% identity over an entire length of the amino acid sequence set forth in SEQ ID NO:6; and a nucleotide sequence encoding a peptide comprising an amino acid sequence having at least about 90% identity over an entire length of the amino acid sequence set forth in SEQ ID NO:8.

In one embodiment, the nucleic acid molecule comprises a nucleotide sequence encoding the amino acid sequence selected from the group consisting of SEQ ID NO:2, SEQ ID NO:4, SEQ ID NO:6 and SEQ ID NO:8.

In one embodiment, the nucleic acid molecule comprises a nucleotide sequence selected from the group consisting of: the nucleotide sequence having at least about 90% identity over an entire length of the nucleic acid sequence set forth in SEQ ID NO:1; the nucleotide sequence having at least about 90% identity over an entire length of the nucleic acid sequence set forth in SEQ ID NO:3; the nucleotide sequence having at least about 90% identity over an entire length of the nucleic acid sequence set forth in SEQ ID NO:5; and the nucleotide sequence having at least about 90% identity over an entire length of the nucleic acid sequence set forth in SEQ ID NO:7.

In one embodiment, the nucleic acid molecule comprises the nucleic acid sequence selected from the group consisting of SEQ ID NO:1, SEQ ID NO:3, SEQ ID NO:5 and SEQ ID NO:7.

In one embodiment, administering includes at least one of electroporation and injection.

### BRIEF DESCRIPTION OF THE DRAWINGS

FIG. 1A and FIG. 1B depict a comparison of SARS-CoV-2, SARS-CoV and MERS-CoV spike glycoproteins. FIG. 1A depicts representative amino acid alignment of coronavirus spike proteins including 11 SARS-CoV-2 sequences with mutations (GISAID). Grey bars indicates identical amino acids and colored bars represent mutations relative to Wuhan-Hu-1. Receptor binding domain (RBD), Cleavage Site, Fusion Peptide and Transmembrane domains are indicated in red. FIG. 1B depicts schematic representation of design of SARS-CoV-2 DNA vaccine antigen, in comparison with similar betacoronaviruses, SARS-CoV and MERS-CoV spike glycoproteins. Structural models for SARS-CoV-2, SARS and MERS glycoproteins with one chain represented as cartoon and two chains represented as surface. RBD of SARS-CoV-2 is colored yellow.

FIG. 2A through FIG. 2D depict depicts the design and expression of COVID-19 synthetic DNA vaccine constructs. FIG. 2A depicts schematic diagram of COVID-19 synthetic DNA vaccine constructs, pGX9501 (matched) and pGX9503 (outlier (OL)) containing the IgE leader sequence and SARS-CoV-2 spike protein insert. FIG. 2B depicts representative RT-PCR assay of RNA extract from COS-7 cells transfected with pGX9501 & pGX9503. Extracted RNA was analyzed by RT-PCR using PCR assays designed for each target and for COS-7 β-Actin mRNA, used as an internal expression normalization gene. Delta C_{T} (Δ C_{T}) was calculated as the C_{T} of the target minus the C_{T} of β-Actin for each transfection concentration and is plotted against the log of the mass of pDNA transfected.

FIG. 2C depicts representative analysis of in vitro expression of Spike protein after transfection of 293T cells with pGX9501, pGX9503 or MOCK plasmid by Western blot. 293T cell lysates were resolved on a gel and probed with a polyclonal anti-SARS Spike Protein. Blots were stripped then probed with an anti-β-actin loading control. FIG. 2D depicts representative in vitro immunofluorescent staining of 293T cells transfected with 3 μg/well of pGX9501, pGX9503 or pVax (empty control vector). Expression of Spike protein was measured with polyclonal anti-SARS Spike Protein IgG and anti-IgG secondary (green). Cell nuclei were counterstained with DAPI (blue). Images were captured using ImageXpress Pico automated cell imaging system.

FIG. 3 depicts an IgG binding screen of a panel of SARS-CoV-2 and SARS-CoV antigens using serum from INO-4800-treated mice. BALB/c mice were immunized on Day 0 with 25 μg INO-4800 or pVAX-empty vector (Control) as described in the methods. Protein antigen binding of IgG at 1:50 and 1:250 serum dilutions from mice at day 14. Data shown represent mean OD450 nm values (mean+SD) for each group of 4 mice.

FIG. 4A through FIG. 4D depict the humoral responses to SARS-CoV-2 S1+2 and S RBD protein antigen in BALB/c mice after a single dose of INO-4800 BALB/c mice were immunized on day 0 with indicated doses of INO-4800 or pVAX-empty vector as described in the methods. FIG. 4A depicts representative SARS-CoV-2 S1+2 protein antigen binding of IgG in serial serum dilutions from mice at day 14. FIG. 4B depicts representative serum IgG binding endpoint titers to SARS-CoV-2 S1+2 protein. FIG. 4C depicts representative SARS-CoV-2 RBD protein antigen binding of IgG in serial serum dilutions from mice at day 14. FIG. 4D depicts representative serum IgG binding endpoint titers to SARS-CoV-2 RBD protein. Data representative of 2 independent experiments. Data shown represent mean OD450 nm values (mean+SD) for each group of 8 mice (FIG. 4A and FIG. 4B) and 5 mice (FIG. 4C and FIG. 4D).

FIG. 5A through FIG. 5C depict serum IgG from INO-4800 immunized mice compete with ACE2 receptor for SARS-CoV-2 Spike protein binding. FIG. 5A depicts representative soluble ACE2 receptor binds to CoV-2 full-length spike with an EC₅₀ of 0.025 μg/ml. FIG. 5B depicts representative purified serum IgG from BALB/c mice after second immunization with INO-4800 yields significant competition against ACE2 receptor. Serum IgG samples from the animals were run in triplicate. FIG. 5C depicts representative IgGs purified from n=5 mice day 14 post second immunization with INO-4800 show significant competition against ACE2 receptor binding to SARS-CoV-2 S1+2 protein. The soluble ACE2 concentration for the competition assay is ˜0.1 μg/ml.

FIG. 6 depicts IgGs purified from n=5 mice day 14 post second immunization with INO-4800 show competition against ACE2 receptor binding to SARS-CoV-2 Spike protein compared to pooled naïve mice IgGs. Naïve mice were run in a single column. Vaccinated mice were run in duplicate. If error bars are not visible, error is smaller than the data point.

FIG. 7A and FIG. 7B depict humoral responses to SARS-CoV-2 in Hartley guinea pigs after a single dose of INO-4800. Hartley guinea pigs mice were immunized on Day 0 with 100 μg INO-4800 or pVAX-empty vector as described in the methods. FIG. 7A depicts representative SARS-CoV-2 S protein antigen binding of IgG in serial serum dilutions at day 0 and 14. Data shown represent mean OD450 nm values (mean+SD) for the 5 guinea pigs. FIG. 7B depicts representative serum IgG binding titers (mean±SD) to SARS-CoV-2 S protein at day 14. P=0.0079, Mann-Whitney test.

FIG. 8A and FIG. 8B depict serum from INO-4800 immunized guinea pigs mediates the inhibition of ACE-2 binding to SARS-CoV-2 S protein. Hartley guinea pigs were immunized on Day 0 and 14 with 100 μg INO-4800 or pVAX-empty vector as described in the methods. FIG. 8A depicts representative day 28 collected serum (diluted 1:20) was added SARS-CoV-2 coated wells prior to the addition of serial dilutions of ACE-2 protein. Detection of ACE-2 binding to SARS-CoV-2 S protein was measured. Sera collected from 5 INO-4800-treated and 3 pVAX-treated animals were used in this experiment. FIG. 8B depicts representative serial dilutions of guinea pig serum collected on day 21 were added to SARS-CoV-2 coated wells prior to the addition of ACE-2 protein. Detection of ACE-2 binding to SARS-CoV-2 S protein was measured. Sera collected from 4 INO-4800-treated and 5 pVAX-treated guinea pigs was used in this experiment.

FIG. 9A through FIG. 9D depict detection of SARS-CoV-2 S protein-reactive antibodies in the BAL of INO-4800 immunized animals. FIG. 9A depicts representative results for BALB/c mice were immunized on days 0 and 14 with INO-4800 or pVAX and BAL collected at day 21. FIG. 9B depicts representative results for BALB/c mice were immunized on days 0 and 14 with INO-4800 or pVAX and BAL collected at day 21. FIG. 9C depicts representative results for hartley guinea pigs were immunized on days 0, 14 and 21 with INO-4800 or pVAX and BAL collected at day 42. FIG. 9D depicts representative results for hartley guinea pigs were immunized on days 0, 14 and 21 with INO-4800 or pVAX and BAL collected at day 42. Bronchoalveolar lavage fluid was assayed for SARS-CoV-2 Spike protein-specific antibodies by ELISA. Data are presented as endpoint titers (FIG. 9A and FIG. 9C), and BAL dilution curves with raw OD 450 nm values (FIG. 9B and FIG. 9D). (FIG. 9A and FIG. 9C) bars represent the average of each group and error bars the standard deviation. **p<0.01 by Mann-Whitney U test. Data are representative of one experiment for each species with n=5/group

FIG. 10A through FIG. 10C depict rapid induction of T cell responses in BALB/c mice post-administration of INO-4800. BALB/c mice (n=5/group) were immunized with 2.5 or 10 μg INO-4800. T cell responses were analyzed in the animals on days 4, 7, 10 for FIG. 10A and FIG. 10B, and day 14 for FIG. 10C. FIG. 10A depicts representative results for T cell responses that were measured by IFN-γ ELISpot in splenocytes stimulated for 20 hours with overlapping peptide pools spanning the SARS-CoV-2 Spike proteins. FIG. 10B depicts representative results for T cell responses that were measured by IFN-γ ELISpot in splenocytes stimulated for 20 hours with overlapping peptide pools spanning the SARS-CoV Spike proteins. FIG. 10C depicts representative results for T cell responses that were measured by IFN-γ ELISpot in splenocytes stimulated for 20 hours with overlapping peptide pools spanning the MERS-CoV Spike proteins. Bars represent the mean+SD.

FIG. 11A through FIG. 11F depict ELISpot images of IFN-γ+ mouse splenocytes after stimulation with SARS-CoV-2 and SARS antigens. Mice were immunized on day 0 and splenocytes harvested at the indicated time points. IFNγ-secreting cells in the spleens of immunized animals were enumerated via ELISpot assay. FIG. 11A depicts representative images showing SARS-CoV-2 specific IFNγ spot forming units in the splenocyte population at day 4 post-immunization. FIG. 11B depicts representative images showing SARS-CoV-2 specific IFNγ spot forming units in the splenocyte population at day 7 post-immunization. FIG. 11C depicts representative images showing SARS-CoV-2 specific IFNγ spot forming units in the splenocyte population at day 10 post-immunization. FIG. 11D depicts representative images showing SARS-CoV-specific IFNγ spot forming units in the splenocyte population at day 4 post-immunization. FIG. 11E depicts representative images showing SARS-CoV-specific IFNγ spot forming units in the splenocyte population at day 7 post-immunization. FIG. 11F depicts representative images showing SARS-CoV-specific IFNγ spot forming units in the splenocyte population at day 10 post-immunization. Images were captured by ImmunoSpot CTL reader.

FIG. 12A and FIG. 12B depict flow cytometric analysis of T cell populations producing IFN-γ upon SARS-CoV-2 S protein stimulation. Splenocytes harvested from BALB/c and C57BL/6 mice 14 days after pVAX or INO-4800 treatment were made into single cell suspensions. The cells were stimulated for 6 hours with SARS-CoV-2 overlapping peptide pools. FIG. 12A depicts representative CD4+ and CD8+ T cell gating strategy; singlets were gated on (i), then lymphocytes (ii) followed by live CD45+ cells (iii). Next CD3+ cells were gated (iv) and from that population CD4+ (v) and CD8+ (vi) T-cells were gated. IFNγ+ cells were gated from each of the CD4+ (vii) and CD8+ (viii) T-cell populations. FIG. 12B depicts representative percentage of CD4+ and CD8+ T cells producing IFNγ is depicted. Bars represent mean+SD. 4 BALB/c and 4 C57BL/6 mice were used in this study. * p<0.05, Mann Whitney test.

FIG. 13A and FIG. 13B depict T cell epitope mapping after INO-4800 administration to BALB/c mice. Splenocytes were stimulated for 20 hours with SARS-CoV-2 peptide matrix pools. FIG. 13A depicts representative T cell responses following stimulation with matrix mapping SARS-CoV-2 peptide pools. Bars represent the mean+SD.

FIG. 13B depicts representative map of the SARS-CoV-2 Spike protein and identification of immunodominant peptides in BALB/c mice. A known immunodominant SARS-CoV HLA-A2 epitope is included for comparison.

FIG. 14 depicts a schematic representation of pAI-4800 overview for post-exposure immunotherapeutic protection against COVID-19 disease progression.

FIG. 15 depicts a schematic representation of COVID-19 immunotherapy phase I study design.

FIG. 16 depicts representative rhesus macaque groups.

FIG. 17 depicts representative COVID-19 immunotherapy platform Phase 2b efficacy study design.

FIG. 18 depicts representative CD8+ T cell infiltration to site of infection following DNA vaccination against HPV (Trimble et al., 2015, Lancet, 386: 2078-2088).

FIG. 19A and FIG. 19B depicts representative results demonstrating protective efficacy of MERS DNA vaccine in rhesus macaques. FIG. 19A depicts representative results demonstrating protection following IM delivery in rhesus macaques using a 3-dose immunization regimen (Muthumani et al., 2015, Sci Transl Med, 7:301ra132). FIG. 19B depicts representative results demonstrating protection following ID delivery in rhesus macaques using a 2-dose immunization regimen.

FIG. 20A and FIG. 20B depicts representative humoral and cellular immune responses in participants receiving the MERS DNA Vaccine GLS-5300 in comparison with responses from MERS convalescent patients. FIG. 20A depicts representative total IgG antibody endpoint titers measured by ELISA. FIG. 20B depicts representative T cell responses measured by IFNγ ELISpot assay (Modjarrad et al, 2019, Lancet Infectious Disease, 19: P1013-1022).

FIG. 21 depicts representative results demonstrating rapid induction of T cell responses in BALB/c mice post-administration of INO-4800. BALB/c mice (n=5/group) were immunized with 2.5 or 10 μg INO-4800. T cell responses were analyzed in the animals on days 4, 7, 10. T cell responses were measured by IFN-γ ELISpot in splenocytes stimulated for 20 hours with overlapping peptide pools spanning the SARS-CoV-2.

FIG. 22A through FIG. 22D depicts representative total antibody responses against SARS-CoV-2 Spike and RBD proteins in mice and guinea pigs. FIG. 22A depicts representative endpoint titers in mice against full length Spike. FIG. 22B depicts representative endpoint titers in mice against RBD. FIG. 22C depicts representative endpoint titers in guinea pigs against full length Spike. FIG. 22D depicts representative endpoint titers in guinea pigs against RBD.

FIG. 23 depicts representative results demonstrating neutralizing antibody responses against SARS-CoV-2 live virus in mice and guinea pigs following SARS-CoV-2 DNA immunizations.

FIG. 24A and FIG. 24B depicts representative humoral and cellular immune responses in rhesus macaques following vaccination with INO-4800. FIG. 24A depicts representative antibody responses against SARS-CoV-2 S1+S2 ECD, 51, S2, and RBD proteins. FIG. 24B depicts representative T cell responses against SARS-CoV-2 Spike peptide pools.

### DETAILED DESCRIPTION OF THE INVENTION

The present invention relates to an immunogenic composition comprising an intravenous immunoglobulin (IVIG) against COVID. COVID-19 is a new and highly pathogenic virus, only emerging in 2019, and thus, the vaccine described herein is one of the first vaccines to target COVID-19. Accordingly, the vaccine provides a treatment for this new and pathogenic virus, for which prior treatment did not exist and potential for a pandemic remains.

In one embodiment, the IVIG is extracted from a biological sample, such as a plasma, blood, or a combination thereof, from a subject having been diagnosed with COVID-19, or having been infected with SARS-CoV-2. In one embodiment, the subject is a subject who has been vaccinated against COVID-19. In one embodiment, the subject has been vaccinated against COVID-19. In one embodiment, the subject has been vaccinated with a nucleic acid molecule encoding a SARS-CoV-2 antigen. In one embodiment, the subject has been cured of COVID-19. Thus, the IVIG can be extracted from a biological sample, such as a plasma, blood, or a combination thereof, from a subject post-exposure to SARS-CoV-2.

In one embodiment, the immunogenic composition comprising IVIG against COVID is administered in combination with a nucleic acid molecule encoding a SARS-CoV-2 antigen, fragment thereof, or variant thereof. The SARS-CoV-2 antigen can be a SARS-CoV-2 consensus spike antigen. The SARS-CoV-2 consensus spike antigen can be derived from the sequences of spike antigens from strains of SARS-CoV-2, and thus, the SARS-CoV-2 consensus spike antigen is unique. The SARS-CoV-2 consensus spike antigen can be an outlier spike antigen, having a greater amino acid sequence divergence from other SARS-CoV-2 spike proteins. Accordingly, the vaccine of the present invention is widely applicable to multiple strains of SARS-CoV-2 because of the unique sequences of the SARS-CoV-2 consensus spike antigen. These unique sequences allow the vaccine to be universally protective against multiple strains of SARS-CoV-2, including genetically diverse variants of SARS-CoV-2.

The vaccine can be used to protect against and treat any number of strains of SARS-CoV-2. The vaccine can elicit both humoral and cellular immune responses that target the SARS-CoV-2 spike antigen. The vaccine can elicit neutralizing antibodies and immunoglobulin G (IgG) antibodies that are reactive with the SARS-CoV-2 spike antigen. The vaccine can also elicit CD8⁺ and CD4⁺ T cell responses that are reactive to the SARS-CoV-2 spike antigen and produce interferon-gamma (IFN-γ), tumor necrosis factor alpha (TNF-α), and interleukin-2 (IL-2).

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of” the embodiments or elements presented herein, whether explicitly set forth or not.

“Adjuvant” as used herein means any molecule added to the vaccine described herein to enhance the immunogenicity of the antigen.

“Antibody” as used herein means an antibody of classes IgG, IgM, IgA, IgD or IgE, or fragments, fragments or derivatives thereof, including Fab, F(ab′)₂, Fd, and single chain antibodies, diabodies, bispecific antibodies, bifunctional antibodies and derivatives thereof. The antibody can be an antibody isolated from the serum sample of mammal, a polyclonal antibody, affinity purified antibody, or mixtures thereof which exhibits sufficient binding specificity to a desired epitope or a sequence derived therefrom.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered.

“Complement” or “complementary” as used herein means Watson-Crick (e.g., A-T/U and C-G) or Hoogsteen base pairing between nucleotides or nucleotide analogs of nucleic acid molecules.

“Consensus” or “Consensus Sequence” as used herein may mean a synthetic nucleic acid sequence, or corresponding polypeptide sequence, constructed based on analysis of an alignment of multiple subtypes of a particular antigen. The sequence may be used to induce broad immunity against multiple subtypes, serotypes, or strains of a particular antigen. Synthetic antigens, such as fusion proteins, may be manipulated to generate consensus sequences (or consensus antigens).

“Electroporation,” “electro-permeabilization,” or “electro-kinetic enhancement” (“EP”) as used interchangeably herein means the use of a transmembrane electric field pulse to induce microscopic pathways (pores) in a bio-membrane; their presence allows biomolecules such as plasmids, oligonucleotides, siRNA, drugs, ions, and water to pass from one side of the cellular membrane to the other.

“Fragment” as used herein means a nucleic acid sequence or a portion thereof that encodes a polypeptide capable of eliciting an immune response in a mammal. The fragments can be DNA fragments selected from at least one of the various nucleotide sequences that encode protein fragments set forth below.

“Fragment” or “immunogenic fragment” with respect to polypeptide sequences means a polypeptide capable of eliciting an immune response in a mammal that cross reacts with a full length wild type strain COVID-19 antigen or the immunoglobulin. Fragments of consensus proteins can comprise at least 10%, at least 20%, at least 30%, at least 40%, at least 50%, at least 60%, at least 70%, at least 80%, at least 90% or at least 95% of a consensus protein. In some embodiments, fragments of consensus proteins can comprise at least 20 amino acids or more, at least 30 amino acids or more, at least 40 amino acids or more, at least 50 amino acids or more, at least 60 amino acids or more, at least 70 amino acids or more, at least 80 amino acids or more, at least 90 amino acids or more, at least 100 amino acids or more, at least 110 amino acids or more, at least 120 amino acids or more, at least 130 amino acids or more, at least 140 amino acids or more, at least 150 amino acids or more, at least 160 amino acids or more, at least 170 amino acids or more, at least 180 amino acids or more, at least 190 amino acids or more, at least 200 amino acids or more, at least 210 amino acids or more, at least 220 amino acids or more, at least 230 amino acids or more, or at least 240 amino acids or more of a consensus protein.

As used herein, the term “genetic construct” refers to the DNA or RNA molecules that comprise a nucleotide sequence which encodes a protein. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed.

“Identical” or “identity” as used herein in the context of two or more nucleic acids or polypeptide sequences, means that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

“Immune response” as used herein means the activation of a host's immune system, e.g., that of a mammal, in response to the introduction of antigen. The immune response can be in the form of a cellular or humoral response, or both.

“Nucleic acid” or “oligonucleotide” or “polynucleotide” as used herein means at least two nucleotides covalently linked together. The depiction of a single strand also defines the sequence of the complementary strand. Thus, a nucleic acid also encompasses the complementary strand of a depicted single strand. Many variants of a nucleic acid can be used for the same purpose as a given nucleic acid. Thus, a nucleic acid also encompasses substantially identical nucleic acids and complements thereof. A single strand provides a probe that can hybridize to a target sequence under stringent hybridization conditions. Thus, a nucleic acid also encompasses a probe that hybridizes under stringent hybridization conditions.

Nucleic acids can be single stranded or double stranded, or can contain portions of both double stranded and single stranded sequence. The nucleic acid can be DNA, both genomic and cDNA, RNA, or a hybrid, where the nucleic acid can contain combinations of deoxyribo- and ribo-nucleotides, and combinations of bases including uracil, adenine, thymine, cytosine, guanine, inosine, xanthine hypoxanthine, isocytosine and isoguanine. Nucleic acids can be obtained by chemical synthesis methods or by recombinant methods.

“Operably linked” as used herein means that expression of a gene is under the control of a promoter with which it is spatially connected. A promoter can be positioned 5′ (upstream) or 3′ (downstream) of a gene under its control. The distance between the promoter and a gene can be approximately the same as the distance between that promoter and the gene it controls in the gene from which the promoter is derived. As is known in the art, variation in this distance can be accommodated without loss of promoter function.

A “peptide,” “protein,” or “polypeptide” as used herein can mean a linked sequence of amino acids and can be natural, synthetic, or a modification or combination of natural and synthetic.

“Promoter” as used herein means a synthetic or naturally-derived molecule which is capable of conferring, activating or enhancing expression of a nucleic acid in a cell. A promoter can comprise one or more specific transcriptional regulatory sequences to further enhance expression and/or to alter the spatial expression and/or temporal expression of same. A promoter can also comprise distal enhancer or repressor elements, which can be located as much as several thousand base pairs from the start site of transcription. A promoter can be derived from sources including viral, bacterial, fungal, plants, insects, and animals. A promoter can regulate the expression of a gene component constitutively or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter.

“Signal peptide” and “leader sequence” are used interchangeably herein and refer to an amino acid sequence that can be linked at the amino terminus of a COVID-19 protein set forth herein. Signal peptides/leader sequences typically direct localization of a protein. Signal peptides/leader sequences used herein preferably facilitate secretion of the protein from the cell in which it is produced. Signal peptides/leader sequences are often cleaved from the remainder of the protein, often referred to as the mature protein, upon secretion from the cell. Signal peptides/leader sequences are linked at the N terminus of the protein.

“Subject” as used herein can mean a mammal that wants to or is in need of being immunized with the herein described vaccine. The mammal can be a human, chimpanzee, dog, cat, horse, cow, mouse, or rat.

“Substantially identical” as used herein can mean that a first and second amino acid sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or more amino acids. Substantially identical can also mean that a first nucleic acid sequence and a second nucleic acid sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or more nucleotides.

“Treatment” or “treating,” as used herein can mean protecting of an animal from a disease through means of preventing, suppressing, repressing, or completely eliminating the disease. Preventing the disease involves administering a vaccine of the present invention to an animal prior to onset of the disease. Suppressing the disease involves administering a vaccine of the present invention to an animal after induction of the disease but before its clinical appearance. Repressing the disease involves administering a vaccine of the present invention to an animal after clinical appearance of the disease.

“Variant” used herein with respect to a nucleic acid means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

Variant can further be defined as a peptide or polypeptide that differs in amino acid sequence by the insertion, deletion, or conservative substitution of amino acids, but retain at least one biological activity. Representative examples of “biological activity” include the ability to be bound by a specific antibody or to promote an immune response. Variant can also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. Substitution of amino acids having similar hydrophilicity values can result in peptides retaining biological activity, for example immunogenicity, as is understood in the art. Substitutions can be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant may be a nucleic acid sequence that is substantially identical over the full length of the full gene sequence or a fragment thereof. The nucleic acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof. A variant may be an amino acid sequence that is substantially identical over the full length of the amino acid sequence or fragment thereof. The amino acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

“Vector” as used herein means a nucleic acid sequence containing an origin of replication. A vector can be a viral vector, bacteriophage, bacterial artificial chromosome or yeast artificial chromosome. A vector can be a DNA or RNA vector. A vector can be a self-replicating extrachromosomal vector, and preferably, is a DNA plasmid.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2. COVID-IVIG

The invention is based, in part, on the development of an Intravenous Immunoglobulin for the treatment or prevention of SARS-CoV-2 infection, or a disease or disorder associated therewith, such as COVID-19.

Intravenous immunoglobulin (IVIG) generally consists of a non-specific immunoglobulin solution obtained by combining plasma from a plurality of individuals. In one embodiment, the COVID intravenous immunoglobulin (COVID-IVIG) of the invention is a solution of immunoglobulins obtained from plasma collected from subjects who were vaccinated with a nucleic acid molecule encoding a SARS-CoV-2 antigen, as described in detail elsewhere herein. The solution therefore contains a relatively high titre of anti-SARS-CoV-2 antibodies. In one embodiment, the COVID-IVIG comprises a solution of immunoglobulins obtained from plasma collected from subjects who were diagnosed as having COVID-19 prior to being vaccinated with a nucleic acid molecule encoding a SARS-CoV-2 antigen.

In some embodiments, the invention relates to a composition comprising an immunoglobulin. Immunoglobulins include, but are not limited to, polyclonal, monoclonal, chimeric and humanized antibodies, and include the following classes: IgA, IgG, IgM, IgD, and IgE. Immunoglobulins generally comprise two identical heavy chains and light chains. However, the immunoglobulins can also include fragments, such as the single chain immunoglobulins, as well as protein dimers, polymers or aggregates.

In some embodiments, the immunoglobulin is capable of eliciting an immune response in a mammal against one or more SARS-CoV-2 and/or COVID-19 strains.

The immunoglobulin is extracted from a biological sample obtained from a subject having the immunoglobulin. For example, the immunoglobulin can be extracted from a biological sample obtained from a subject that was vaccinated against COVID-19, treated for SARS-CoV-2 and/or COVID-19, diagnosed as having SARS-CoV-2 and/or COVID-19, recovered from COVID-19, cured of COVID-19, or any combination thereof. The immunoglobulin can be extracted from a biological sample obtained from a subject post-exposure and post-treatment of SARS-CoV-2 and/or COVID-19.

In some embodiments, the immunoglobulins are obtained from subjects who are further negative for anti-HIV1/2, negative for anti-HTLV1/2, negative for anti-HCV, negative for HBsAg, negative for serologic test for syphilis, negative for HIV NAT, negative for HCV NAT, negative for WNV NAT, negative for Hepatitis A virus NAT, negative for Hepatitis B virus NAT, negative for Parvovirus B19 NAT, and any combination thereof. Thus, in some embodiments, the biological sample from which the immunoglobulin is extracted, is negative for anti-HIV1/2, negative for anti-HTLV1/2, negative for anti-HCV, negative for HBsAg, negative for serologic test for syphilis, negative for HIV NAT, negative for HCV NAT, negative for WNV NAT, negative for Hepatitis A virus NAT, negative for Hepatitis B virus NAT, negative for Parvovirus B19 NAT, and any combination thereof.

The biological sample may be of any biological tissue or fluid. Frequently the sample will be a “clinical sample” which is a sample derived from a patient. Such samples include, but are not limited to, bodily fluids which may or may not contain cells, e.g., blood (e.g., whole blood, serum or plasma), urine, saliva, tissue or fine needle biopsy samples, tissue sample obtained during surgical resection, and archival samples with known diagnosis, treatment and/or outcome history. The biological sample may contain any biological material suitable for extracting the desired immunoglobulin, and may comprise cellular and/or non-cellular material obtained from the individual. A biological sample can be obtained by appropriate methods, such as, by way of examples, blood draw, fluid draw, biopsy, or surgical resection. Examples of such samples include, but are not limited to blood, lymph, urine, gastrointestinal fluid, semen, and biopsies. Samples that are liquid in nature are referred to herein as “bodily fluids.” Body samples may be obtained from a patient by a variety of techniques including, for example, by scraping or swabbing an area or by using a needle to aspirate bodily fluids. Methods for collecting various body samples are well known in the art.

Provided herein are immunogenic compositions comprising a COVID-IVIG. The vaccine can be used to protect against any number of strains of COVID-19, any number of strains of SARS-CoV-2, thereby treating, preventing, and/or protecting against SARS-CoV-2 based pathologies, such as COVID-19. The immunogenic compositions can significantly induce an immune response of a subject administered the immunogenic composition, thereby protecting against and treating COVID-19 and/or SARS-CoV-2 infection.

The vaccine can induce a humoral immune response in the subject administered the vaccine. The induced humoral immune response can be specific for a SARS-CoV-2 antigen. The induced humoral immune response can be reactive with the SARS-CoV-2 antigen. The humoral immune response can be induced in the subject administered the vaccine by about 1.5-fold to about 16-fold, about 2-fold to about 12-fold, or about 3-fold to about 10-fold. The humoral immune response can be induced in the subject administered the vaccine by at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about 15.0-fold, at least about 15.5-fold, or at least about 16.0-fold.

The humoral immune response induced by the vaccine can include an increased level of neutralizing antibodies associated with the subject administered the vaccine as compared to a subject not administered the vaccine. The neutralizing antibodies can be specific for a SARS-CoV-2 antigen. The neutralizing antibodies can be reactive with a SARS-CoV-2 antigen. The neutralizing antibodies can provide prevention, protection against, and/or treatment of COVID-19 and/or SARS-CoV-2 infection and its associated pathologies in the subject administered the vaccine.

The humoral immune response induced by the vaccine can include an increased level of IgG antibodies associated with the subject administered the vaccine as compared to a subject not administered the vaccine. These IgG antibodies can be specific for the COVID-19 antigen and/or the immunoglobulin. These IgG antibodies can be reactive with the COVID-19 antigen and/or the immunoglobulin. Preferably, the humoral response is cross-reactive against two or more strains of the COVID-19. The level of IgG antibody associated with the subject administered the vaccine can be increased by about 1.5-fold to about 16-fold, about 2-fold to about 12-fold, or about 3-fold to about 10-fold as compared to the subject not administered the vaccine. The level of IgG antibody associated with the subject administered the vaccine can be increased by at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about 15.0-fold, at least about 15.5-fold, or at least about 16.0-fold as compared to the subject not administered the vaccine.

The vaccine can induce a cellular immune response in the subject administered the vaccine. The induced cellular immune response can be specific for the COVID-19 antigen and/or the immunoglobulin. The induced cellular immune response can be reactive to the COVID-19 antigen and/or the immunoglobulin. Preferably, the cellular response is cross-reactive against two or more strains of the COVID-19. The induced cellular immune response can include eliciting a CD8⁺ T cell response. The elicited CD8⁺ T cell response can be reactive with the COVID-19 antigen and/or the immunoglobulin. The elicited CD8⁺ T cell response can be polyfunctional. The induced cellular immune response can include eliciting a CD8⁺ T cell response, in which the CD8⁺ T cells produce interferon-gamma (IFN-γ), tumor necrosis factor alpha (TNF-α), interleukin-2 (IL-2), or a combination of IFN-γ and TNF-α.

The induced cellular immune response can include an increased CD8⁺ T cell response associated with the subject administered the vaccine as compared to the subject not administered the vaccine. The CD8⁺ T cell response associated with the subject administered the vaccine can be increased by about 2-fold to about 30-fold, about 3-fold to about 25-fold, or about 4-fold to about 20-fold as compared to the subject not administered the vaccine. The CD8⁺ T cell response associated with the subject administered the vaccine can be increased by at least about 1.5-fold, at least about 2.0-fold, at least about 3.0-fold, at least about 4.0-fold, at least about 5.0-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about 15.0-fold, at least about 16.0-fold, at least about 17.0-fold, at least about 18.0-fold, at least about 19.0-fold, at least about 20.0-fold, at least about 21.0-fold, at least about 22.0-fold, at least about 23.0-fold, at least about 24.0-fold, at least about 25.0-fold, at least about 26.0-fold, at least about 27.0-fold, at least about 28.0-fold, at least about 29.0-fold, or at least about 30.0-fold as compared to the subject not administered the vaccine.

The induced cellular immune response can include an increased frequency of CD3⁺CD8⁺ T cells that produce IFN-γ. The frequency of CD3⁺CD8⁺IFN-γ⁺ T cells associated with the subject administered the vaccine can be increased by at least about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold as compared to the subject not administered the vaccine.

The induced cellular immune response can include an increased frequency of CD3⁺CD8⁺ T cells that produce TNF-α. The frequency of CD3⁺CD8⁺ TNF-α⁺ T cells associated with the subject administered the vaccine can be increased by at least about 2-fold, 3-fold, 4-fold, 5-fold, 6-fold, 7-fold, 8-fold, 9-fold, 10-fold, 11-fold, 12-fold, 13-fold, or 14-fold as compared to the subject not administered the vaccine.

The induced cellular immune response can include an increased frequency of CD3⁺CD8⁺ T cells that produce IL-2. The frequency of CD3⁺CD8⁺IL-2⁺ T cells associated with the subject administered the vaccine can be increased by at least about 0.5-fold, 1.0-fold, 1.5-fold, 2.0-fold, 2.5-fold, 3.0-fold, 3.5-fold, 4.0-fold, 4.5-fold, or 5.0-fold as compared to the subject not administered the vaccine.

The induced cellular immune response can include an increased frequency of CD3⁺CD8⁺ T cells that produce both IFN-γ and TNF-α. The frequency of CD3⁺CD8⁺IFN-γ⁺ TNF-α⁺ T cells associated with the subject administered the vaccine can be increased by at least about 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 110-fold, 120-fold, 130-fold, 140-fold, 150-fold, 160-fold, 170-fold, or 180-fold as compared to the subject not administered the vaccine.

The cellular immune response induced by the vaccine can include eliciting a CD4⁺ T cell response. The elicited CD4⁺ T cell response can be reactive with the COVID-19 antigen and/or the immunoglobulin. The elicited CD4⁺ T cell response can be polyfunctional. The induced cellular immune response can include eliciting a CD4⁺ T cell response, in which the CD4⁺ T cells produce IFN-γ, TNF-α, IL-2, or a combination of IFN-γ and TNF-α.

The induced cellular immune response can include an increased frequency of CD3⁺CD4⁺ T cells that produce IFN-γ. The frequency of CD3⁺CD4⁺IFN-γ⁺ T cells associated with the subject administered the vaccine can be increased by at least about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold as compared to the subject not administered the vaccine.

The induced cellular immune response can include an increased frequency of CD3⁺CD4⁺ T cells that produce TNF-α. The frequency of CD3⁺CD4⁺ TNF-α⁺ T cells associated with the subject administered the vaccine can be increased by at least about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, or 22-fold as compared to the subject not administered the vaccine.

The induced cellular immune response can include an increased frequency of CD3⁺CD4⁺ T cells that produce IL-2. The frequency of CD3⁺CD4⁺IL-2⁺ T cells associated with the subject administered the vaccine can be increased by at least about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, 30-fold, 31-fold, 32-fold, 33-fold, 34-fold, 35-fold, 36-fold, 37-fold, 38-fold, 39-fold, 40-fold, 45-fold, 50-fold, 55-fold, or 60-fold as compared to the subject not administered the vaccine.

The induced cellular immune response can include an increased frequency of CD3⁺CD4⁺ T cells that produce both IFN-γ and TNF-α. The frequency of CD3⁺CD4⁺IFN-γ⁺ TNF-α⁺ associated with the subject administered the vaccine can be increased by at least about 2-fold, 2.5-fold, 3.0-fold, 3.5-fold, 4.0-fold, 4.5-fold, 5.0-fold, 5.5-fold, 6.0-fold, 6.5-fold, 7.0-fold, 7.5-fold, 8.0-fold, 8.5-fold, 9.0-fold, 9.5-fold, 10.0-fold, 10.5-fold, 11.0-fold, 11.5-fold, 12.0-fold, 12.5-fold, 13.0-fold, 13.5-fold, 14.0-fold, 14.5-fold, 15.0-fold, 15.5-fold, 16.0-fold, 16.5-fold, 17.0-fold, 17.5-fold, 18.0-fold, 18.5-fold, 19.0-fold, 19.5-fold, 20.0-fold, 21-fold, 22-fold, 23-fold 24-fold, 25-fold, 26-fold, 27-fold, 28-fold, 29-fold, 30-fold, 31-fold, 32-fold, 33-fold, 34-fold, or 35-fold as compared to the subject not administered the vaccine.

The vaccine of the present invention can have features required of effective vaccines such as being safe so the vaccine itself does not cause illness or death; is protective against illness resulting from exposure to live pathogens such as viruses or bacteria; induces neutralizing antibody to prevent invention of cells; induces protective T cells against intracellular pathogens; and provides ease of administration, few side effects, biological stability, and low cost per dose.

The vaccine can further induce an immune response when administered to different tissues such as the muscle or skin. The vaccine can further induce an immune response when administered via electroporation, or injection, or subcutaneously, or intramuscularly.

### 3. COVID-19 ANTIGEN

In some embodiments, the present invention relates to methods of generating a COVID-IVIG comprising isolating immunoglobulins from a subject who has been vaccinated against SARS-CoV-2. In one embodiment, the subject has been vaccinated with a nucleic acid molecule encoding a SARS-CoV-2 antigen, a variant thereof, or a fragment thereof.

In one embodiment, the subject has been vaccinated with a peptide vaccine comprising a COVID-19 antigenic peptide, a variant thereof, a fragment thereof, a COVID-19 antigenic protein, a variant thereof, a fragment thereof, an immunoglobulin, a variant thereof, a fragment thereof, or any combination thereof.

In some embodiments, the present invention relates to methods of treating, protecting against, and/or preventing SARS-CoV-2 infection, or a disease or disorder associated with SARS-CoV-2 infection in a subject in need thereof by administering a combination of a COVID-IVIG and a nucleic acid molecule encoding a SARS-CoV-2 antigen.

In some embodiments, the present invention relates to a combination of a COVID-IVIG and a vaccine comprising a nucleic acid sequence encoding a COVID-19 antigenic peptide, wherein the COVID-IVIG is generated from subjects who were vaccinated with the nucleic acid sequence encoding a COVID-19 antigenic peptide.

Therefore, in some embodiments, the invention relates to immunogenic compositions comprising a nucleic acid molecule encoding a SARS-CoV-2 antigen and their use in method of generating a COVID-IVIG and in methods of treating, protecting against, and/or preventing SARS-CoV-2 infection, or a disease or disorder associated with SARS-CoV-2 infection. In one embodiment, the nucleic acid sequence can be DNA, RNA, cDNA, a variant thereof, a fragment thereof, or a combination thereof. The nucleic acid sequence can also include additional sequences that encode linker, leader, or tag sequences that are linked to the nucleotide sequence encoding the COVID-19 antigen.

As described above, the vaccine comprises a COVID-19 antigen, a fragment thereof, a variant thereof, or a combination thereof. Coronaviruses, including COVID-19, are encapsulated by a membrane and have a type 1 membrane glycoprotein known as spike (S) protein, which forms protruding spikes on the surface of the coronavirus. The spike protein facilitates binding of the coronavirus to proteins located on the surface of a cell, for example, the metalloprotease amino peptidase N, and mediates cell-viral membrane fusion. In particular, the spike protein contains an S1 subunit that facilitates binding of the coronavirus to cell surface proteins. Accordingly, the S1 subunit of the spike protein controls which cells are infected by the coronavirus. The spike protein also contains a S2 subunit, which is a transmembrane subunit that facilitates viral and cellular membrane fusion. Accordingly, the COVID-19 antigen can comprise a COVID-19 spike protein, a S1 subunit of a COVID-19 spike protein, or a S2 subunit of a COVID-19 spike protein.

Upon binding cell surface proteins and membrane fusion, the coronavirus enters the cell and its singled-stranded RNA genome is released into the cytoplasm of the infected cell. The singled-stranded RNA genome is a positive strand and thus, can be translated into a RNA polymerase, which produces additional viral RNAs that are minus strands. Accordingly, the COVID-19 antigen can also be a COVID-19 RNA polymerase.

The viral minus RNA strands are transcribed into smaller, subgenomic positive RNA strands, which are used to translate other viral proteins, for example, nucleocapsid (N) protein, envelope (E) protein, and matrix (M) protein. Accordingly, the COVID-19 antigen can comprise a COVID-19 nucleocapsid protein, a COVID-19 envelope protein, or a C OVID-19 matrix protein.

The viral minus RNA strands can also be used to replicate the viral genome, which is bound by nucleocapsid protein. Matrix protein, along with spike protein, is integrated into the endoplasmic reticulum of the infected cell. Together, the nucleocapsid protein bound to the viral genome and the membrane-embedded matrix and spike proteins are budded into the lumen of the endoplasmic reticulum, thereby encasing the viral genome in a membrane. The viral progeny are then transported by golgi vesicles to the cell membrane of the infected cell and released into the extracellular space by endocytosis.

In some embodiments, the COVID-19 antigen can be a COVID-19 spike protein, a COVID-19 RNA polymerase, a COVID-19 nucleocapsid protein, a COVID-19 envelope protein, a COVID-19 matrix protein, a fragment thereof, a variant thereof, or a combination thereof. The COVID-19 antigen can be a consensus antigen derived from two or more COVID-19 spike antigens, two or more COVID-19 RNA polymerases, two or more COVID-19 nucleocapsid proteins, two or more envelope proteins, two or more matrix proteins, or a combination thereof. The COVID-19 consensus antigen can be modified for improved expression. Modification can include codon optimization, RNA optimization, addition of a kozak sequence for increased translation initiation, and/or the addition of an immunoglobulin leader sequence to increase the immunogenicity of the COVID-19 antigen. In some embodiments the COVID-19 antigen includes an IgE leader, which can be the amino acid sequence set forth in SEQ ID NO:9.

### SARS-CoV-2 Spike Antigen

The SARS-CoV-2 antigen can be a SARS-CoV-2 spike antigen, a fragment thereof, a variant thereof, or a combination thereof. The SARS-CoV-2 spike antigen is capable of eliciting an immune response in a mammal against one or more SARS-CoV-2 strains. The SARS-CoV-2 spike antigen can comprise an epitope(s) that makes it particularly effective as an immunogen against which an anti-SARS-CoV-2 immune response can be induced.

The SARS-CoV-2 spike antigen can be a consensus sequence derived from two or more strains of SARS-CoV-2. The SARS-CoV-2 spike antigen can comprise a consensus sequence and/or modification(s) for improved expression. Modification can include codon optimization, RNA optimization, addition of a kozak sequence for increased translation initiation, and/or the addition of an immunoglobulin leader sequence to increase the immunogenicity of the SARS-CoV-2 spike antigen. The SARS-CoV-2 spike antigen can comprise a signal peptide such as an immunoglobulin signal peptide, for example, but not limited to, an immunoglobulin E (IgE) or immunoglobulin (IgG) signal peptide. In some embodiments, the SARS-CoV-2 spike antigen can comprise a hemagglutinin (HA) tag. The SARS-CoV-2 spike antigen can be designed to elicit stronger and broader cellular and/or humoral immune responses than a corresponding codon optimized spike antigen.

The SARS-CoV-2 consensus spike antigen can be the amino acid sequence SEQ ID NO:2. In some embodiments, the SARS-CoV-2 consensus spike antigen can be the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence set forth in SEQ ID NO:2.

The SARS-CoV-2 consensus spike antigen can be the nucleic acid sequence SEQ ID NO:1, which encodes SEQ ID NO:2. In some embodiments, the SARS-CoV-2 consensus spike antigen can be the nucleic acid sequence having at least about 80%, 81%, 82%, 83%, 84%, 85%, 86%, 87%, 88%, 89%, 90%, 91%, 92%, 93%, 94%, 95%, 96%, 97%, 98%, 99% or 100% identity over an entire length of the nucleic acid sequence set forth in SEQ ID NO:1. In other embodiments, the SARS-CoV-2 consensus spike antigen can be the nucleic acid sequence that encodes the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence set forth in SEQ ID NO:2.

The SARS-CoV-2 consensus spike antigen can be operably linked to an IgE leader sequence. The SARS-CoV-2 consensus spike antigen operably linked to an IgE leader sequence can be the amino acid sequence SEQ ID NO:4. In some embodiments, the SARS-CoV-2 consensus spike antigen can be the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence set forth in SEQ ID NO:4.

The SARS-CoV-2 consensus spike antigen can be the nucleic acid sequence SEQ ID NO:3, which encodes SEQ ID NO:4. In some embodiments, the SARS-CoV-2 consensus spike antigen can be the nucleic acid sequence having at least about 80%, 81%, 82%, 83%, 84%, 85%, 86%, 87%, 88%, 89%, 90%, 91%, 92%, 93%, 94%, 95%, 96%, 97%, 98%, 99% or 100% identity over an entire length of the nucleic acid sequence set forth in SEQ ID NO:3. In other embodiments, the SARS-CoV-2 consensus spike antigen can be the nucleic acid sequence that encodes the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence set forth in SEQ ID NO:4.

Immunogenic fragments of SEQ ID NO:2 or SEQ ID NO:4 can be provided. Immunogenic fragments can comprise at least 60%, at least 65%, at least 70%, at least 75%, at least 80%, at least 85%, at least 90%, at least 95%, at least 96%, at least 97%, at least 98% or at least 99% of SEQ ID NO:2 or SEQ ID NO:4. In some embodiments, immunogenic fragments include a leader sequence, such as for example an immunoglobulin leader, such as the IgE leader. In some embodiments, immunogenic fragments are free of a leader sequence.

Immunogenic fragments of proteins with amino acid sequences homologous to immunogenic fragments of SEQ ID NO:2 or SEQ ID NO:4 can be provided. Such immunogenic fragments can comprise at least 60%, at least 65%, at least 70%, at least 75%, at least 80%, at least 85%, at least 90%, at least 95%, at least 96%, at least 97%, at least 98% or at least 99% of proteins that are 95% homologous to SEQ ID NO:2 or SEQ ID NO:4. Some embodiments relate to immunogenic fragments that have 96% homology to the immunogenic fragments of protein sequences herein. Some embodiments relate to immunogenic fragments that have 97% homology to the immunogenic fragments of protein sequences herein. Some embodiments relate to immunogenic fragments that have 98% homology to the immunogenic fragments of protein sequences herein. Some embodiments relate to immunogenic fragments that have 99% homology to the immunogenic fragments of protein sequences herein. In some embodiments, immunogenic fragments include a leader sequence, such as for example an immunoglobulin leader, such as the IgE leader. In some embodiments, immunogenic fragments are free of a leader sequence.

Some embodiments relate to immunogenic fragments of SEQ ID NO:1 or SEQ ID NO:3. Immunogenic fragments can be at least 60%, at least 65%, at least 70%, at least 75%, at least 80%, at least 85%, at least 90%, at least 95%, at least 96%, at least 97%, at least 98% or at least 99% of SEQ ID NO:1 or SEQ ID NO:3. Immunogenic fragments can be at least 95%, at least 96%, at least 97% at least 98% or at least 99% homologous to fragments of SEQ ID NO:1 or SEQ ID NO:3. In some embodiments, immunogenic fragments include sequences that encode a leader sequence, such as for example an immunoglobulin leader, such as the IgE leader. In some embodiments, fragments are free of coding sequences that encode a leader sequence.

### Outlier SARS-CoV-2 Spike Antigen

The SARS-CoV-2 antigen can be an outlier SARS-CoV-2 spike antigen, a fragment thereof, a variant thereof, or a combination thereof. The outlier SARS-CoV-2 spike antigen is capable of eliciting an immune response in a mammal against one or more SARS-CoV-2 strains. The outlier SARS-CoV-2 spike antigen can comprise an epitope(s) that makes it particularly effective as an immunogen against which an anti-SARS-CoV-2 immune response can be induced.

The outlier SARS-CoV-2 spike antigen can be a consensus sequence derived from two or more strains of SARS-CoV-2. The outlier SARS-CoV-2 spike antigen can comprise a consensus sequence and/or modification(s) for improved expression. Modification can include codon optimization, RNA optimization, addition of a kozak sequence for increased translation initiation, and/or the addition of an immunoglobulin leader sequence to increase the immunogenicity of the outlier SARS-CoV-2 spike antigen. The outlier SARS-CoV-2 spike antigen can comprise a signal peptide such as an immunoglobulin signal peptide, for example, but not limited to, an immunoglobulin E (IgE) or immunoglobulin (IgG) signal peptide. The outlier SARS-CoV-2 spike antigen can be designed to elicit stronger and broader cellular and/or humoral immune responses than a corresponding spike antigen.

The outlier SARS-CoV-2 spike antigen can be the amino acid sequence SEQ ID NO:6. In some embodiments, the outlier SARS-CoV-2 spike antigen can be the amino acid sequence having at least about 90%, 91%, 92%, 93%, 94%, 95%, 96%, 97%, 98%, 99%, or 100% identity over an entire length of the amino acid sequence set forth in SEQ ID NO:6.

The outlier SARS-CoV-2 spike antigen can be the nucleic acid sequence SEQ ID NO:5, which encodes SEQ ID NO:6. In some embodiments, the outlier SARS-CoV-2 spike antigen can be the nucleic acid sequence having at least about 90%, 91%, 92%, 93%, 94%, 95%, 96%, 97%, 98%, 99% or 100% identity over an entire length of the nucleic acid sequence set forth in SEQ ID NO:5. In other embodiments, the outlier SARS-CoV-2 spike antigen can be the nucleic acid sequence that encodes the amino acid sequence having at least about 90%, 91%, 92%, 93%, 94%, 95%, 96%, 97%, 98%, 99%, or 100% identity over an entire length of the amino acid sequence set forth in SEQ ID NO:6.

The outlier SARS-CoV-2 spike antigen can be operably linked to an IgE leader sequence. The outlier SARS-CoV-2 spike antigen operably linked to an IgE leader sequence can be the amino acid sequence SEQ ID NO:8. In some embodiments, the outlier SARS-CoV-2 spike antigen can be the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence set forth in SEQ ID NO:8.

The outlier SARS-CoV-2 spike antigen can be the nucleic acid sequence SEQ ID NO:7, which encodes SEQ ID NO:8. In some embodiments, the outlier SARS-CoV-2 spike antigen can be the nucleic acid sequence having at least about 80%, 81%, 82%, 83%, 84%, 85%, 86%, 87%, 88%, 89%, 90%, 91%, 92%, 93%, 94%, 95%, 96%, 97%, 98%, 99% or 100% identity over an entire length of the nucleic acid sequence set forth in SEQ ID NO:7. In other embodiments, the outlier SARS-CoV-2 spike antigen can be the nucleic acid sequence that encodes the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence set forth in SEQ ID NO:8.

Immunogenic fragments of SEQ ID NO:6 or SEQ ID NO:8 can be provided. Immunogenic fragments can comprise at least 60%, at least 65%, at least 70%, at least 75%, at least 80%, at least 85%, at least 90%, at least 95%, at least 96%, at least 97%, at least 98% or at least 99% of SEQ ID NO:6 or SEQ ID NO:8. In some embodiments, immunogenic fragments include a leader sequence, such as for example an immunoglobulin leader, such as the IgE leader. In some embodiments, immunogenic fragments are free of a leader sequence.

Immunogenic fragments of proteins with amino acid sequences homologous to immunogenic fragments of SEQ ID NO:6 or SEQ ID NO:8 can be provided. Such immunogenic fragments can comprise at least 60%, at least 65%, at least 70%, at least 75%, at least 80%, at least 85%, at least 90%, at least 95%, at least 96%, at least 97%, at least 98% or at least 99% of proteins that are 95% homologous to SEQ ID NO:6 or SEQ ID NO:8. Some embodiments relate to immunogenic fragments that have 96% homology to the immunogenic fragments of protein sequences herein. Some embodiments relate to immunogenic fragments that have 97% homology to the immunogenic fragments of protein sequences herein. Some embodiments relate to immunogenic fragments that have 98% homology to the immunogenic fragments of protein sequences herein. Some embodiments relate to immunogenic fragments that have 99% homology to the immunogenic fragments of protein sequences herein. In some embodiments, immunogenic fragments include a leader sequence, such as for example an immunoglobulin leader, such as the IgE leader. In some embodiments, immunogenic fragments are free of a leader sequence.

Some embodiments relate to immunogenic fragments of SEQ ID NO:5 or SEQ ID NO:7. Immunogenic fragments can be at least 60%, at least 65%, at least 70%, at least 75%, at least 80%, at least 85%, at least 90%, at least 95%, at least 96%, at least 97%, at least 98% or at least 99% of SEQ ID NO:5 or SEQ ID NO:7. Immunogenic fragments can be at least 95%, at least 96%, at least 97% at least 98% or at least 99% homologous to fragments of SEQ ID NO:5 or SEQ ID NO:7. In some embodiments, immunogenic fragments include sequences that encode a leader sequence, such as for example an immunoglobulin leader, such as the IgE leader. In some embodiments, fragments are free of coding sequences that encode a leader sequence.

a. Vector

In one embodiment, the immunogenic composition can comprise one or more vectors that include a nucleic acid encoding the SARS-CoV-2 antigen. The one or more vectors can be capable of expressing the antigen. The vector can have a nucleic acid sequence containing an origin of replication. The vector can be a plasmid, bacteriophage, bacterial artificial chromosome or yeast artificial chromosome. The vector can be either a self-replicating extrachromosomal vector or a vector which integrates into a host genome.

The one or more vectors can be an expression construct, which is generally a plasmid that is used to introduce a specific gene into a target cell. Once the expression vector is inside the cell, the protein that is encoded by the gene is produced by the cellular-transcription and translation machinery ribosomal complexes. The plasmid is frequently engineered to contain regulatory sequences that act as enhancer and promoter regions and lead to efficient transcription of the gene carried on the expression vector. The vectors of the present invention express large amounts of stable messenger RNA, and therefore proteins.

The vectors may have expression signals such as a strong promoter, a strong termination codon, adjustment of the distance between the promoter and the cloned gene, and the insertion of a transcription termination sequence and a PTIS (portable translation initiation sequence).

(1) Expression Vectors

The vector can be a circular plasmid or a linear nucleic acid. The circular plasmid and linear nucleic acid are capable of directing expression of a particular nucleotide sequence in an appropriate subject cell. The vector can have a promoter operably linked to the antigen-encoding nucleotide sequence, which may be operably linked to termination signals. The vector can also contain sequences required for proper translation of the nucleotide sequence. The vector comprising the nucleotide sequence of interest may be chimeric, meaning that at least one of its components is heterologous with respect to at least one of its other components. The expression of the nucleotide sequence in the expression cassette may be under the control of a constitutive promoter or of an inducible promoter, which initiates transcription only when the host cell is exposed to some particular external stimulus. In the case of a multicellular organism, the promoter can also be specific to a particular tissue or organ or stage of development.

(2) Circular and Linear Vectors

The vector may be a circular plasmid, which may transform a target cell by integration into the cellular genome or exist extrachromosomally (e.g., autonomous replicating plasmid with an origin of replication).

The vector can be pVAX, pcDNA3.0, or provax, or any other expression vector capable of expressing DNA encoding the antigen and enabling a cell to translate the sequence to an antigen that is recognized by the immune system.

Also provided herein is a linear nucleic acid vaccine, or linear expression cassette (“LEC”), that is capable of being efficiently delivered to a subject via electroporation and expressing one or more desired antigens. The LEC may be any linear DNA devoid of any phosphate backbone. The DNA may encode one or more antigens. The LEC may contain a promoter, an intron, a stop codon, and/or a polyadenylation signal. The expression of the antigen may be controlled by the promoter. The LEC may not contain any antibiotic resistance genes and/or a phosphate backbone. The LEC may not contain other nucleic acid sequences unrelated to the desired antigen gene expression.

(3) Promoter, Intron, Stop Codon, and Polyadenylation Signal

The vector may have a promoter. A promoter may be any promoter that is capable of driving gene expression and regulating expression of the isolated nucleic acid. Such a promoter is a cis-acting sequence element required for transcription via a DNA dependent RNA polymerase, which transcribes the antigen sequence described herein. Selection of the promoter used to direct expression of a heterologous nucleic acid depends on the particular application. The promoter may be positioned about the same distance from the transcription start in the vector as it is from the transcription start site in its natural setting. However, variation in this distance may be accommodated without loss of promoter function.

The promoter may be operably linked to the nucleic acid sequence encoding the antigen and signals required for efficient polyadenylation of the transcript, ribosome binding sites, and translation termination. The promoter may be a CMV promoter, SV40 early promoter, SV40 later promoter, metallothionein promoter, murine mammary tumor virus promoter, Rous sarcoma virus promoter, polyhedrin promoter, or another promoter shown effective for expression in eukaryotic cells.

The vector may include an enhancer and an intron with functional splice donor and acceptor sites. The vector may contain a transcription termination region downstream of the structural gene to provide for efficient termination. The termination region may be obtained from the same gene as the promoter sequence or may be obtained from different genes.

b. Excipients and Other Components of the Compositions

The immunogenic composition of the invention may further comprise a pharmaceutically acceptable excipient. The pharmaceutically acceptable excipient can be functional molecules such as vehicles, carriers, or diluents. The pharmaceutically acceptable excipient can be a transfection facilitating agent, which can include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The transfection facilitating agent is a polyanion, polycation, including poly-L-glutamate (LGS), or lipid. The transfection facilitating agent is poly-L-glutamate, and the poly-L-glutamate may be present in the vaccine at a concentration less than 6 mg/ml. The transfection facilitating agent may also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the genetic construct. The DNA plasmid vaccines may also include a transfection facilitating agent such as lipids, liposomes, including lecithin liposomes or other liposomes known in the art, as a DNA-liposome mixture (see for example WO9324640), calcium ions, viral proteins, polyanions, polycations, or nanoparticles, or other known transfection facilitating agents. The transfection facilitating agent is a polyanion, polycation, including poly-L-glutamate (LGS), or lipid. Concentration of the transfection agent in the vaccine is less than 4 mg/ml, less than 2 mg/ml, less than 1 mg/ml, less than 0.750 mg/ml, less than 0.500 mg/ml, less than 0.250 mg/ml, less than 0.100 mg/ml, less than 0.050 mg/ml, or less than 0.010 mg/ml.

The pharmaceutically acceptable excipient can be an adjuvant. The adjuvant can be other genes that are expressed in an alternative plasmid or are delivered as proteins in combination with the plasmid above in the vaccine. The adjuvant may be selected from the group consisting of: α-interferon (IFN-α), β-interferon (IFN-β), γ-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHC, CD80, CD86 including IL-15 having the signal sequence deleted and optionally including the signal peptide from IgE. The adjuvant can be IL-12, IL-15, IL-28, CTACK, TECK, platelet derived growth factor (PDGF), TNFα, TNFβ, GM-CSF, epidermal growth factor (EGF), IL-1, IL-2, IL-4, IL-5, IL-6, IL-10, IL-12, IL-18, or a combination thereof.

Other genes that can be useful as adjuvants include those encoding: MCP-1, MIP-1a, MIP-1p, IL-8, RANTES, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, IL-22, nerve growth factor, vascular endothelial growth factor, Fas, TNF receptor, Flt, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof.

The vaccine may further comprise a genetic vaccine facilitator agent as described in U.S. Ser. No. 021,579 filed Apr. 1, 1994, which is fully incorporated by reference.

The vaccine can be formulated according to the mode of administration to be used. An injectable vaccine pharmaceutical composition can be sterile, pyrogen free and particulate free. An isotonic formulation or solution can be used. Additives for isotonicity can include sodium chloride, dextrose, mannitol, sorbitol, and lactose. The vaccine can comprise a vasoconstriction agent. The isotonic solutions can include phosphate buffered saline. Vaccine can further comprise stabilizers including gelatin and albumin. The stabilizers can allow the formulation to be stable at room or ambient temperature for extended periods of time, including LGS or polycations or polyanions.

### 4. METHOD OF GENERATING A COVID-IVIG

Provided herein are methods of generating an intravenous immunoglobulin (IVIG) of the invention. In one embodiment, the IVIG is a COVID-IVIG, purified from a biological sample from one or more host subject vaccinated with a nucleic acid molecule encoding a SARS-CoV-2 antigen, as described elsewhere herein. In one embodiment, the one or more host subject have been diagnosed with SARS-CoV-2 infection and/or COVID-19. In one embodiment, administration of a nucleic acid molecule encoding a SARS-CoV-2 antigen to the host subject induces or elicits an immune response in the subject. In one embodiment, administration of a nucleic acid molecule encoding a SARS-CoV-2 antigen to the host subject can induces or elicits the generation of at least one anti-SARS-CoV-2 immunoglobulin in the subject. In one embodiment, the anti-SARS-CoV-2 immunoglobulin(s) elicited in one or more host subjects are then isolated and purified for administration to a recipient subject as a COVID-IVIG.

### 5. METHOD OF TREATMENT

Provided herein are methods of treating, protecting against, and/or preventing disease in a subject in need thereof by administering an intravenous immunoglobulin purified from a host subject vaccinated with nucleic acid molecule encoding a SARS-CoV-2 antigen (COVID-IVIG) to the recipient subject. In one embodiment, the host subject has been diagnosed with SARS-CoV-2 infection or COVID-19. In one embodiment, administration of the COVID-IVIG to the recipient subject can induce or elicit an immune response in the recipient subject. The induced immune response can be used to treat, prevent, and/or protect against disease, for example, pathologies relating to COVID-19 and/or SARS-CoV-2 infection. In some embodiments, the induced immune response provides the subject administered the COVID-IVIG resistance to one or more SARS-CoV-2 strains. Thus, in some embodiments, the present invention provides methods of treating, protecting against, and/or preventing COVID-19 in a subject in need thereof by administering a COVID-IVIG to the subject.

In some embodiments, provided herein are methods of treating, protecting against, and/or preventing disease in a subject in need thereof by administering a combination of 1) an intravenous immunoglobulin purified from a host subject vaccinated with nucleic acid molecule encoding a SARS-CoV-2 antigen (COVID-IVIG) and 2) a vaccine comprising a nucleic acid molecule encoding a SARS-CoV-2 antigen, or fragment or variant thereof, to the subject. Administration of the combination of 1) a COVID-IVIG and 2) a vaccine comprising a nucleic acid molecule encoding a SARS-CoV-2 antigen, or fragment or variant thereof, to the subject can induce or elicit an immune response in the recipient subject. In some embodiments, the induced immune response provides the subject administered the combination of 1) a COVID-IVIG and 2) a vaccine comprising a nucleic acid molecule encoding a SARS-CoV-2 antigen, or fragment or variant thereof, resistance to one or more SARS-CoV-2 or COVID-19 strains. The induced immune response can be used to treat, prevent, and/or protect against disease, for example, pathologies relating to COVID-19 and/or SARS-CoV-2 infection. Thus, in some embodiments, the present invention provides methods of treating, protecting against, and/or preventing COVID-19 in a subject in need thereof by administering a combination of 1) a COVID-IVIG and 2) a vaccine comprising a nucleic acid molecule encoding a SARS-CoV-2 antigen, or fragment or variant thereof, to the subject.

The present invention also relates, in part, to methods of inducing an immune response against SARS-Cov-2 in a subject in need thereof by administering a COVID-IVIG alone, or in combination with a vaccine comprising a nucleic acid molecule encoding a SARS-CoV-2 antigen, or fragment or variant thereof, to the subject. The present invention further relates, in part, to methods of protecting a subject in need thereof from a SARS-CoV-2 infection, or a disease or disorder associated therewith, by administering a COVID-IVIG alone, or in combination with a vaccine comprising a nucleic acid molecule encoding a SARS-CoV-2 antigen, or fragment or variant thereof, to the subject. The present invention also provides, in part, methods of protecting a subject in need thereof from developing COVID-19, or a disease or disorder associated therewith, or symptoms thereof, by administering a COVID-IVIG alone, or in combination with a vaccine comprising a nucleic acid molecule encoding a SARS-CoV-2 antigen, or fragment or variant thereof, to the subject. In various embodiments, the present invention provides methods of protecting a subject infected with SARS-CoV-2 from developing COVID-19 or symptoms thereof by administering a COVID-IVIG alone, or in combination with a vaccine comprising a nucleic acid molecule encoding a SARS-CoV-2 antigen, or fragment or variant thereof, to the subject. Thus, the present invention further provides, in part, methods of alleviating or treating COVID-19 or symptoms thereof in a subject in need thereof by administering a COVID-IVIG alone, or in combination with a vaccine comprising a nucleic acid molecule encoding a SARS-CoV-2 antigen, or fragment or variant thereof, to the subject. The present invention also provides, in part, methods of preventing or reducing SARS-CoV-2 or COVID-19 progression in a subject in need thereof by administering a COVID-IVIG alone, or in combination with a vaccine comprising a nucleic acid molecule encoding a SARS-CoV-2 antigen, or fragment or variant thereof, to the subject. The present invention also provides, in part, methods of preventing or reducing SARS-CoV-2 transmission in a subject in need thereof by administering a COVID-IVIG alone, or in combination with a vaccine comprising a nucleic acid molecule encoding a SARS-CoV-2 antigen, or fragment or variant thereof, to the subject.

The induced immune response can include an induced humoral immune response and/or an induced cellular immune response. The humoral immune response can be induced by about 1.5-fold to about 16-fold, about 2-fold to about 12-fold, or about 3-fold to about 10-fold. The induced humoral immune response can include IgG antibodies and/or neutralizing antibodies that are reactive to the antigen. The induced cellular immune response can include a CD8⁺ T cell response, which is induced by about 2-fold to about 30-fold, about 3-fold to about 25-fold, or about 4-fold to about 20-fold.

The vaccine dose can be between 1 μg to 10 mg active component/kg body weight/time, and can be 20 μg to 10 mg component/kg body weight/time. The vaccine can be administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. The number of vaccine doses for effective treatment can be 1, 2, 3, 4, 5, 6, 7, 8, 9, or 10.

### 6. USE IN COMBINATION

In some embodiments, the present invention provides a method of treating, protecting against, and/or preventing SARS-CoV-2 infection, or a disease or disorder associated with SARS-CoV-2 infection in a subject in need thereof by administering a combination of a COVID-IVIG and a nucleic acid molecule encoding a SARS-CoV-2 antigen, or fragment or variant thereof.

The COVID-IVIG and nucleic acid molecule encoding a SARS-CoV-2 antigen may be administered using any suitable method such that a combination of the COVID-IVIG and a nucleic acid molecule encoding a SARS-CoV-2 antigen are both present in the subject. In one embodiment, the method may comprise administration of a first composition comprising a COVID-IVIG of the invention by any of the methods described in detail elsewhere herein and administration of a second composition comprising a nucleic acid molecule encoding a SARS-CoV-2 antigen less than 1, less than 2, less than 3, less than 4, less than 5, less than 6, less than 7, less than 8, less than 9 or less than 10 days following administration of the COVID-IVIG. In one embodiment, the method may comprise administration of a first composition comprising a COVID-IVIG of the invention by any of the methods described in detail above and administration of a second composition comprising a nucleic acid molecule encoding a SARS-CoV-2 antigen more than 1, more than 2, more than 3, more than 4, more than 5, more than 6, more than 7, more than 8, more than 9 or more than 10 days following administration of the COVID-IVIG. In one embodiment, the method may comprise administration of a first composition comprising a nucleic acid molecule encoding a SARS-CoV-2 antigen and administration of a second composition comprising a COVID-IVIG of the invention by any of the methods described in detail above less than 1, less than 2, less than 3, less than 4, less than 5, less than 6, less than 7, less than 8, less than 9 or less than 10 days following administration of the nucleic acid molecule encoding a SARS-CoV-2 antigen. In one embodiment, the method may comprise administration of a first composition comprising a nucleic acid molecule encoding a SARS-CoV-2 antigen and administration of a second composition comprising a COVID-IVIG of the invention by any of the methods described in detail above more than 1, more than 2, more than 3, more than 4, more than 5, more than 6, more than 7, more than 8, more than 9 or more than 10 days following administration of the nucleic acid molecule encoding a SARS-CoV-2 antigen. In one embodiment, the method may comprise administration of a first composition comprising a COVID-IVIG of the invention by any of the methods described in detail above and a second composition comprising a nucleic acid molecule encoding a SARS-CoV-2 antigen concurrently. In one embodiment, the method may comprise administration of a single composition comprising a COVID-IVIG of the invention and a nucleic acid molecule encoding a SARS-CoV-2 antigen.

In some embodiments, the present invention provides a method of treating, protecting against, and/or preventing SARS-CoV-2 infection, or a disease or disorder associated with SARS-CoV-2 infection in a subject in need thereof by administering a combination of a COVID-IVIG and a nucleic acid molecule encoding a SARS-CoV-2 antigen in combination with one or more additional therapeutic agent. In one embodiment, the therapeutic agent is an antiviral agent. In one embodiment, the therapeutic is an antibiotic agent.

Non-limiting examples of antibiotics that can be used in combination with the combination of a COVID-IVIG and a nucleic acid molecule encoding a SARS-CoV-2 antigen of the invention include aminoglycosides (e.g., gentamicin, amikacin, tobramycin), quinolones (e.g., ciprofloxacin, levofloxacin), cephalosporins (e.g., ceftazidime, cefepime, cefoperazone, cefpirome, ceftobiprole), antipseudomonal penicillins: carboxypenicillins (e.g., carbenicillin and ticarcillin) and ureidopenicillins (e.g., mezlocillin, azlocillin, and piperacillin), carbapenems (e.g., meropenem, imipenem, doripenem), polymyxins (e.g., polymyxin B and colistin) and monobactams (e.g., aztreonam).

a. Administration

A composition comprising a COVID-IVIG, and a composition comprising a nucleic acid molecule encoding a SARS-CoV-2 antigen, or a composition comprising the combination thereof, can be formulated in accordance with standard techniques well known to those skilled in the pharmaceutical art. Such compositions can be administered in dosages and by techniques well known to those skilled in the medical arts taking into consideration such factors as the age, sex, weight, and condition of the particular subject, and the route of administration. The subject can be a mammal, such as a human, a horse, a cow, a pig, a sheep, a cat, a dog, a rat, or a mouse.

The compositions can be administered prophylactically or therapeutically. In prophylactic administration, the compositions can be administered in an amount sufficient to induce an immune response. In therapeutic applications, the compositions are administered to a subject in need thereof in an amount sufficient to elicit a therapeutic effect. An amount adequate to accomplish this is defined as “therapeutically effective dose.” Amounts effective for this use will depend on, e.g., the particular composition of the treatment regimen administered, the manner of administration, the stage and severity of the disease, the general state of health of the patient, and the judgment of the prescribing physician.

The vaccine can be administered by methods well known in the art as described in Donnelly et al. (Ann. Rev. Immunol. 15:617-648 (1997)); Felgner et al. (U.S. Pat. No. 5,580,859, issued Dec. 3, 1996); Felgner (U.S. Pat. No. 5,703,055, issued Dec. 30, 1997); and Carson et al. (U.S. Pat. No. 5,679,647, issued Oct. 21, 1997), the contents of all of which are incorporated herein by reference in their entirety. In some embodiments, DNA for administration as a vaccine can be complexed to particles or beads that can be administered to an individual, for example, using a vaccine gun. One skilled in the art would know that the choice of a pharmaceutically acceptable carrier, including a physiologically acceptable compound, depends, for example, on the route of administration of the expression vector.

The compositions of the invention can be delivered via a variety of routes. Typical delivery routes include parenteral administration, e.g., intradermal, intramuscular or subcutaneous delivery. Other routes include oral administration, intranasal, and intravaginal routes. For a DNA vaccine in particular, the vaccine can be delivered to the interstitial spaces of tissues of an individual (Felgner et al., U.S. Pat. Nos. 5,580,859 and 5,703,055, the contents of all of which are incorporated herein by reference in their entirety). The compositions can also be administered to muscle, or can be administered via intradermal or subcutaneous injections, or transdermally, such as by iontophoresis. Epidermal administration of the vaccine can also be employed. Epidermal administration can involve mechanically or chemically irritating the outermost layer of epidermis to stimulate an immune response to the irritant (Carson et al., U.S. Pat. No. 5,679,647, the contents of which are incorporated herein by reference in its entirety).

The compositions can also be formulated for administration via the nasal passages. Formulations suitable for nasal administration, wherein the carrier is a solid, can include a coarse powder having a particle size, for example, in the range of about 10 to about 500 microns which is administered in the manner in which snuff is taken, i.e., by rapid inhalation through the nasal passage from a container of the powder held close up to the nose. The formulation can be a nasal spray, nasal drops, or by aerosol administration by nebulizer. The formulation can include aqueous or oily solutions of the compositions.

The compositions can be a liquid preparation such as a suspension, syrup or elixir. The compositions can also be a preparation for parenteral, subcutaneous, intradermal, intramuscular or intravenous administration (e.g., injectable administration), such as a sterile suspension or emulsion.

The compositions can be incorporated into liposomes, microspheres or other polymer matrices (Felgner et al., U.S. Pat. No. 5,703,055; Gregoriadis, Liposome Technology, Vols. Ito III (2nd ed. 1993), the contents of which are incorporated herein by reference in their entirety). Liposomes can consist of phospholipids or other lipids, and can be nontoxic, physiologically acceptable and metabolizable carriers that are relatively simple to make and administer.

The compositions can be administered via electroporation, such as by a method described in U.S. Pat. No. 7,664,545, the contents of which are incorporated herein by reference. The electroporation can be by a method and/or apparatus described in U.S. Pat. Nos. 6,302,874; 5,676,646; 6,241,701; 6,233,482; 6,216,034; 6,208,893; 6,192,270; 6,181,964; 6,150,148; 6,120,493; 6,096,020; 6,068,650; and 5,702,359, the contents of which are incorporated herein by reference in their entirety. The electroporation may be carried out via a minimally invasive device.

The minimally invasive electroporation device (“MID”) may be an apparatus for injecting a composition comprising a nucleic acid molecule described above and associated fluid into body tissue. The device may comprise a hollow needle, DNA cassette, and fluid delivery means, wherein the device is adapted to actuate the fluid delivery means in use so as to concurrently (for example, automatically) inject DNA into body tissue during insertion of the needle into the said body tissue. This has the advantage that the ability to inject the DNA and associated fluid gradually while the needle is being inserted leads to a more even distribution of the fluid through the body tissue. The pain experienced during injection may be reduced due to the distribution of the DNA being injected over a larger area.

The MID may inject the vaccine into tissue without the use of a needle. The MID may inject the vaccine as a small stream or jet with such force that the vaccine pierces the surface of the tissue and enters the underlying tissue and/or muscle. The force behind the small stream or jet may be provided by expansion of a compressed gas, such as carbon dioxide through a micro-orifice within a fraction of a second. Examples of minimally invasive electroporation devices, and methods of using them, are described in published U.S. Patent Application No. 20080234655; U.S. Pat. Nos. 6,520,950; 7,171,264; 6,208,893; 6,009,347; 6,120,493; 7,245,963; 7,328,064; and 6,763,264, the contents of each of which are herein incorporated by reference.

The MID may comprise an injector that creates a high-speed jet of liquid that painlessly pierces the tissue. Such needle-free injectors are commercially available. Examples of needle-free injectors that can be utilized herein include those described in U.S. Pat. Nos. 3,805,783; 4,447,223; 5,505,697; and 4,342,310, the contents of each of which are herein incorporated by reference.

A desired vaccine in a form suitable for direct or indirect electrotransport may be introduced (e.g., injected) using a needle-free injector into the tissue to be treated, usually by contacting the tissue surface with the injector so as to actuate delivery of a jet of the agent, with sufficient force to cause penetration of the vaccine into the tissue. For example, if the tissue to be treated is mucosa, skin or muscle, the agent is projected towards the mucosal or skin surface with sufficient force to cause the agent to penetrate through the stratum corneum and into dermal layers, or into underlying tissue and muscle, respectively.

Needle-free injectors are well suited to deliver vaccines to all types of tissues, particularly to skin and mucosa. In some embodiments, a needle-free injector may be used to propel a liquid that contains the vaccine to the surface and into the subject's skin or mucosa. Representative examples of the various types of tissues that can be treated using the invention methods include pancreas, larynx, nasopharynx, hypopharynx, oropharynx, lip, throat, lung, heart, kidney, muscle, breast, colon, prostate, thymus, testis, skin, mucosal tissue, ovary, blood vessels, or any combination thereof.

The MID may have needle electrodes that electroporate the tissue. By pulsing between multiple pairs of electrodes in a multiple electrode array, for example set up in rectangular or square patterns, provides improved results over that of pulsing between a pair of electrodes. Disclosed, for example, in U.S. Pat. No. 5,702,359 entitled “Needle Electrodes for Mediated Delivery of Drugs and Genes” is an array of needles wherein a plurality of pairs of needles may be pulsed during the therapeutic treatment. In that application, which is incorporated herein by reference as though fully set forth, needles were disposed in a circular array, but have connectors and switching apparatus enabling a pulsing between opposing pairs of needle electrodes. A pair of needle electrodes for delivering recombinant expression vectors to cells may be used. Such a device and system is described in U.S. Pat. No. 6,763,264, the contents of which are herein incorporated by reference. Alternatively, a single needle device may be used that allows injection of the DNA and electroporation with a single needle resembling a normal injection needle and applies pulses of lower voltage than those delivered by presently used devices, thus reducing the electrical sensation experienced by the patient.

The MID may comprise one or more electrode arrays. The arrays may comprise two or more needles of the same diameter or different diameters. The needles may be evenly or unevenly spaced apart. The needles may be between 0.005 inches and 0.03 inches, between 0.01 inches and 0.025 inches; or between 0.015 inches and 0.020 inches. The needle may be 0.0175 inches in diameter. The needles may be 0.5 mm, 1.0 mm, 1.5 mm, 2.0 mm, 2.5 mm, 3.0 mm, 3.5 mm, 4.0 mm, or more spaced apart.

The MID may consist of a pulse generator and a two or more-needle vaccine injectors that deliver the vaccine and electroporation pulses in a single step. The pulse generator may allow for flexible programming of pulse and injection parameters via a flash card operated personal computer, as well as comprehensive recording and storage of electroporation and patient data. The pulse generator may deliver a variety of volt pulses during short periods of time. For example, the pulse generator may deliver three 15 volt pulses of 100 ms in duration. An example of such a MID is the Elgen 1000 system by Inovio Biomedical Corporation, which is described in U.S. Pat. No. 7,328,064, the contents of which are herein incorporated by reference.

The MID may be a CELLECTRA (Inovio Pharmaceuticals, Blue Bell Pa.) device and system, which is a modular electrode system, that facilitates the introduction of a macromolecule, such as a DNA, into cells of a selected tissue in a body or plant. The modular electrode system may comprise a plurality of needle electrodes; a hypodermic needle; an electrical connector that provides a conductive link from a programmable constant-current pulse controller to the plurality of needle electrodes; and a power source. An operator can grasp the plurality of needle electrodes that are mounted on a support structure and firmly insert them into the selected tissue in a body or plant. The macromolecules are then delivered via the hypodermic needle into the selected tissue. The programmable constant-current pulse controller is activated and constant-current electrical pulse is applied to the plurality of needle electrodes. The applied constant-current electrical pulse facilitates the introduction of the macromolecule into the cell between the plurality of electrodes. Cell death due to overheating of cells is minimized by limiting the power dissipation in the tissue by virtue of constant-current pulses. The Cellectra device and system is described in U.S. Pat. No. 7,245,963, the contents of which are herein incorporated by reference.

The MID may be an Elgen 1000 system (Inovio Pharmaceuticals). The Elgen 1000 system may comprise device that provides a hollow needle; and fluid delivery means, wherein the apparatus is adapted to actuate the fluid delivery means in use so as to concurrently (for example automatically) inject fluid, the described vaccine herein, into body tissue during insertion of the needle into the said body tissue. The advantage is the ability to inject the fluid gradually while the needle is being inserted leads to a more even distribution of the fluid through the body tissue. It is also believed that the pain experienced during injection is reduced due to the distribution of the volume of fluid being injected over a larger area.

In addition, the automatic injection of fluid facilitates automatic monitoring and registration of an actual dose of fluid injected. This data can be stored by a control unit for documentation purposes if desired.

It will be appreciated that the rate of injection could be either linear or non-linear and that the injection may be carried out after the needles have been inserted through the skin of the subject to be treated and while they are inserted further into the body tissue.

Suitable tissues into which fluid may be injected by the apparatus of the present invention include tumor tissue, skin or liver tissue but may be muscle tissue.

The apparatus further comprises needle insertion means for guiding insertion of the needle into the body tissue. The rate of fluid injection is controlled by the rate of needle insertion. This has the advantage that both the needle insertion and injection of fluid can be controlled such that the rate of insertion can be matched to the rate of injection as desired. It also makes the apparatus easier for a user to operate. If desired means for automatically inserting the needle into body tissue could be provided.

A user could choose when to commence injection of fluid. Ideally however, injection is commenced when the tip of the needle has reached muscle tissue and the apparatus may include means for sensing when the needle has been inserted to a sufficient depth for injection of the fluid to commence. This means that injection of fluid can be prompted to commence automatically when the needle has reached a desired depth (which will normally be the depth at which muscle tissue begins). The depth at which muscle tissue begins could for example be taken to be a preset needle insertion depth such as a value of 4 mm which would be deemed sufficient for the needle to get through the skin layer.

The sensing means may comprise an ultrasound probe. The sensing means may comprise a means for sensing a change in impedance or resistance. In this case, the means may not as such record the depth of the needle in the body tissue but will rather be adapted to sense a change in impedance or resistance as the needle moves from a different type of body tissue into muscle. Either of these alternatives provides a relatively accurate and simple to operate means of sensing that injection may commence. The depth of insertion of the needle can further be recorded if desired and could be used to control injection of fluid such that the volume of fluid to be injected is determined as the depth of needle insertion is being recorded.

The apparatus may further comprise: a base for supporting the needle; and a housing for receiving the base therein, wherein the base is moveable relative to the housing such that the needle is retracted within the housing when the base is in a first rearward position relative to the housing and the needle extends out of the housing when the base is in a second forward position within the housing. This is advantageous for a user as the housing can be lined up on the skin of a patient, and the needles can then be inserted into the patient's skin by moving the housing relative to the base.

As stated above, it is desirable to achieve a controlled rate of fluid injection such that the fluid is evenly distributed over the length of the needle as it is inserted into the skin. The fluid delivery means may comprise piston driving means adapted to inject fluid at a controlled rate. The piston driving means could for example be activated by a servo motor. However, the piston driving means may be actuated by the base being moved in the axial direction relative to the housing. It will be appreciated that alternative means for fluid delivery could be provided. Thus, for example, a closed container which can be squeezed for fluid delivery at a controlled or non-controlled rate could be provided in the place of a syringe and piston system.

The apparatus described above could be used for any type of injection. It is however envisaged to be particularly useful in the field of electroporation and so it may further comprise means for applying a voltage to the needle. This allows the needle to be used not only for injection but also as an electrode during, electroporation. This is particularly advantageous as it means that the electric field is applied to the same area as the injected fluid. There has traditionally been a problem with electroporation in that it is very difficult to accurately align an electrode with previously injected fluid and so users have tended to inject a larger volume of fluid than is required over a larger area and to apply an electric field over a higher area to attempt to guarantee an overlap between the injected substance and the electric field. Using the present invention, both the volume of fluid injected and the size of electric field applied may be reduced while achieving a good fit between the electric field and the fluid.

### 7. KIT

Provided herein is a kit, which can be used for treating a subject using the method of vaccination described above. The kit can comprise the vaccine.

The kit can also comprise instructions for carrying out the vaccination method described above and/or how to use the kit. Instructions included in the kit can be affixed to packaging material or can be included as a package insert. While instructions are typically written or printed materials, they are not limited to such. Any medium capable of storing instructions and communicating them to an end user is contemplated by this disclosure. Such media include, but are not limited to, electronic storage media (e.g., magnetic discs, tapes, cartridges), optical media (e.g., CD ROM), and the like. As used herein, the term “instructions” can include the address of an internet site which provides instructions.

The present invention has multiple aspects, illustrated by the following non-limiting examples.

### 8. EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1

### Rapid Development of a Synthetic DNA Vaccine for COVID-19

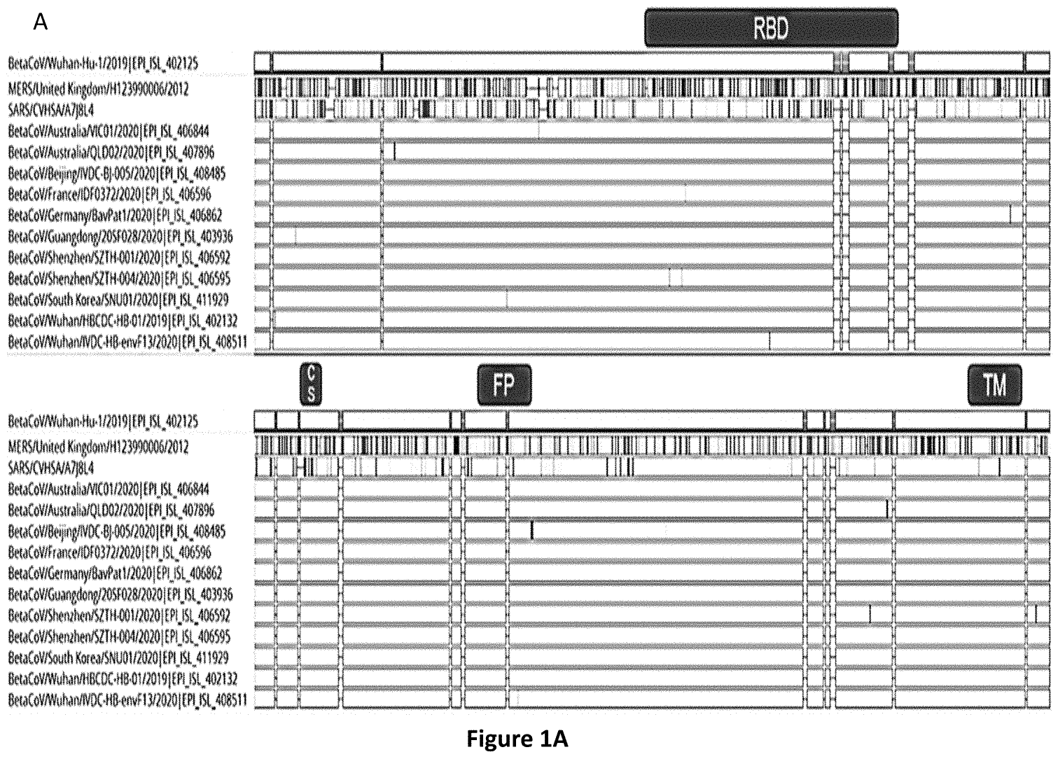

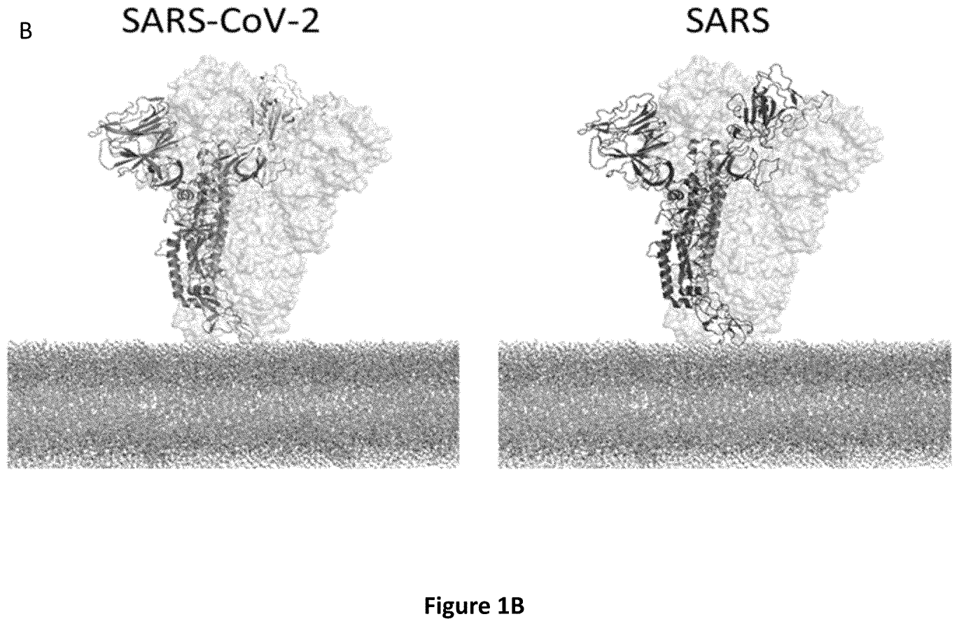

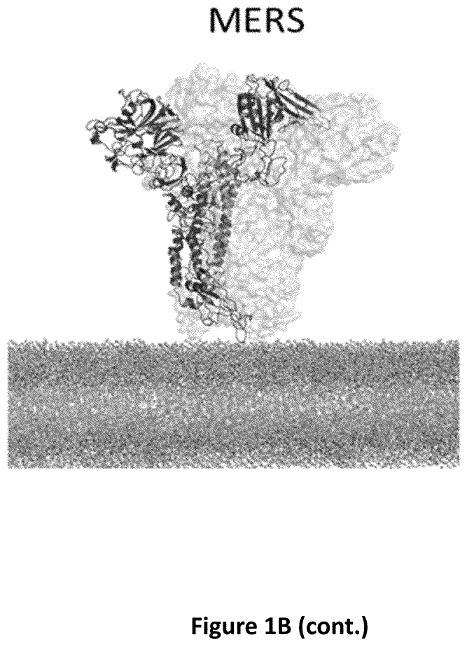

The SARS-CoV-2 spike is most similar in sequence and structure to SARS-CoV spike protein (Wrapp et al., 2020, Science, eabb2507), and shares a global protein fold architecture with the MERS-CoV spike protein (FIG. 1). Unlike glycoproteins of HIV and influenza, the prefusion form of the coronavirus trimeric spike is conformationally dynamic, fully exposing the receptor-binding site infrequently (Kirchdoerfer et al., 2018, Sci Rep, 8:15701). The receptor-binding site is a vulnerable target for neutralizing antibodies. In fact, MERS nAbs targeted at the receptor-binding domain (RBD) tend to have greater neutralizing potency than other epitopes (Wang et al., 2018, J Virol, 92:e02002-17). A recent report demonstrated that one neutralizing anti-SARS antibody could cross-react to the RBD of SARS-CoV-2 (Tian et al., 2020, Emerg Microbes Infect, 9:382-385). These data suggest that the SARS-CoV-2 RBD is an important target for vaccine development. Recent data has revealed SARS-CoV-2 S protein binds the same host receptor, angiotensin-converting enzyme 2 (ACE-2) as SARS-CoV S protein (Wrapp et al., 2020, Science, eabb2507).

Here, the design and initial preclinical testing of COVID-19 synthetic DNA vaccine candidates are described. Expression of the SARS-CoV-2 S antigen RNA and protein are shown after in vitro transfection of COS and 293T cells, respectively with the vaccine candidates. The induction of immunity by the selected immunogen was followed in mice and guinea pigs, measuring SARS-CoV-2 S protein-specific antibody levels in serum and in the lung fluid, and competitive inhibition of ACE2 binding. The INO-4800 vaccine induces cellular and humoral host immune responses that can be observed within days following a single immunization, including cross-reactive responses against SARS-CoV. Taken together, the data demonstrate the immunogenicity of this COVID-19 synthetic DNA vaccine candidate targeting the SARS-CoV-2 S protein, supporting further translational studies to rapidly accelerate the development of this candidate to respond to the current global health crisis.

The novel coronavirus, SARS-CoV-2, and associated COVID-19 disease is rapidly spreading, and has become a global pandemic. Currently, there are no COVID-19 vaccines available, and global dissemination of SARS-CoV-2 may continue until there is a high level of herd immunity within the human population. Here, accelerated preclinical development of a synthetic DNA-based COVID-19 vaccine, INO-4800, is described to combat this emerging infectious disease. Synthetic DNA vaccine design and synthesis was immediately initiated upon public release of the SARS-CoV-2 genome sequences on Jan. 11, 2020. The majority of the in vitro and in vivo studies described herein were executed within 6 weeks of the SARS-CoV-2 genome sequence becoming available. The data support the expression and immunogenicity of the INO-4800 synthetic DNA vaccine candidate in multiple animal models. Humoral and T cells responses were observed in mice after a single dose. In guinea pigs clinical delivery parameters were employed and antibody titers were observed after a single dose.

Halting a rapidly emerging infectious disease requires an orchestrated response from the global health community and requires improved strategies to accelerate vaccine development. In response to the 2019/2020 coronavirus outbreak the highly adaptable synthetic DNA medicine platform was rapidly employed. The design and manufacture of synthetic DNA vaccines for novel antigens is a process in which the target antigen sequence is inserted into a highly characterized and clinically-tested plasmid vector backbone (pGX0001). The construct design and engineering parameters are optimized for in vivo gene expression.

SARS-CoV-2 S protein was chosen as the antigen target. The SARS-CoV-2 S protein is a class I membrane fusion protein, which the major envelope protein on the surface of coronaviruses. Initial studies indicate that SARS-CoV-2 interaction with its host receptor (ACE-2) can be blocked by antibodies (Zhou et al., 2020, Nature, 579:270-273). In vivo immunogenicity studies in both mouse and guinea pig models revealed levels of S protein-reactive IgG in the serum of INO-4800 immunized animals. In addition to full-length S1+S2 and S1, INO-4800 immunization induced RBD binding antibodies, a domain known to be a target for neutralizing antibodies from SARS-CoV convalescent patients (Zhu et al., 2007, Proc Natl Acad Sci USA, 104:12123-12128; He et al., 2005, Virology, 334:74-82). The experiments presented herein further demonstrate the functionality of these antibodies through competitive inhibition of SARS-CoV-2 spike protein binding to the ACE2 receptor in the presence of sera from INO-4800 immunized animals. Importantly, anti-SARS-CoV-2 binding antibodies were detected in lung washes of INO-4800-immunized mice and guinea pigs. The presence of these antibodies in the lungs has the potential to protect against infection of these tissues and prevent LRD, which is associated with the severe cases of COVID-19. In addition to humoral responses, cellular immune responses have been shown to be associated with more favorable recovery in MERS-CoV infection (Zhao et al., 2017, Sci Immunol, 2:eaan5393), and are likely to be important against SARS-CoV infection (Oh et al., 2012, Emerg Microbes Infect, 1:e23). Here, the experiments showed the induction of T cell responses against SARS-CoV-2 as early as day 7 post-vaccine delivery. Rapid cellular responses have the potential to lower viral load and could potentially reduce the spread of SARS-CoV-2 and the associated COVID-19 illness.

In addition to the ability of INO-4800 to rapidly elicit humoral and cellular responses following a single immunization, the synthetic DNA medicine platform has several synergistic characteristics which position it well to respond to disease outbreaks, such as COVID-19. As mentioned previously, the ability to design and synthesize candidate vaccine constructs means that in vitro and in vivo testing can potentially begin within days of receiving the viral sequence, allowing for an accelerated response to vaccine development. The well-defined and established production processes for DNA plasmid manufacture result in a rapid and scalable manufacture process which has the potential to circumvent the complexities of conventional vaccine production in eggs or cell culture. The cost of goods related to DNA manufacture is also significantly lower than currently seen for mRNA-based technologies. A report on the stability profile afforded to through the use of the optimized DNA formulation has recently been published (Tebas et al., 2019, J Infect Dis, 220:400-410). The stability characteristics mean that the DNA drug product is non-frozen and can be stored for 4.5+ years at 2-8° C., room temperature for 1 year and 1 week at 37° C., while maintaining potency at temperatures upwards of 60° C. In the context of a pandemic outbreak, the stability profile of a vaccine plays directly to its ability to be deployed and stockpiled in an efficient and executable manner.

Although vaccine-induced immunopathology has been raised as a potential concern for SARS and MERS vaccine candidates, and possibly for SARS-CoV-2 vaccines, these concerns are likely vaccine-platform dependent and, to-date, no evidence of immune pathogenesis has been reported for MERS DNA vaccines in mice or non-human primate models (Muthumani et al., 2015, Sci Transl Med, 7:301ra13) or SARS DNA vaccine in mice (Yang et al., 2004, Nature, 428:561-564). Lung immunopathology characterized by Th2-related eosinophilia has been reported for whole inactivated virus (IV), recombinant protein, peptide, and/or recombinant viral vector vaccines following SARS-CoV challenge (Tseng et al., 2012, PLoS One, 7:e35421; Iwata-Yoshikawa et al., 2014, J Virol, 88:8597-8614; Bolles et al., 2011, J Virol, 85:12201-12215; Yasui et al., 2008, J Immunol, 181:6337-6348; Wang et al., 2016, ACS Infect Dis, 2:361-376), and more recently in a MERS-CoV challenge model (Agrawal et al., 2016, Hum Vaccin Immunother, 12:2351-2356). However, protective efficacy without lung immunopathology has also been reported for SARS-CoV and MERS-CoV vaccines (Yang et al., 2004, Nature, 428:561-564; Muthumani et al., 2015, Sci Transl Med, 7:301ra132; Luo et al., 2018, Virol Sin 33, 201-204; Qin et al., 2006, Vaccine, 24:1028-1034; Roberts et al., 2010, Viral Immunol 23, 509-519; Deng et al., 2018, Emerg Microbes Infect, 7:60; Zhang et al., 2016, Cell Mol Immunol, 13:180-190; Luke et al., 2016, Sci Transl Med, 8:326ra321; Darnell et al., 2007, J Infect Dis, 196:1329-1338). It is important to note the majority of studies demonstrating CoV vaccine-induced immunopathology utilized the BALB/c mouse, a model known to preferentially develop Th2-type responses. The DNA vaccine platform induces Th1-type immune responses and has demonstrated efficacy without immunopathology in models of respiratory infection, including SARS-CoV (Yang et al., 2004, Nature, 428:561-564), MERS-CoV (Muthumani et al., 2015, Sci Transl Med, 7:301ra132), and RSV (Smith et al., 2017, Vaccine, 35:2840-2847). Additional studies assess vaccine-enhanced disease in INO-4800 immunized animals.

Additional preclinical studies are ongoing to further characterize INO-4800 in small and larger animals. Availability of reagents is a major challenge for development of vaccines against newly emerging infectious diseases, and this limited the ability, in this early stage study, to report on the live virus neutralizing activity of the antibodies in animal models. However, it is reported herein that INO-4800 induced antibodies block SARS-CoV-2 Spike binding to the host receptor ACE2, using a surrogate neutralization assay. Additional studies with live virus neutralizing assays are informative for the investigation of antibody functionality, and demonstrate the ability of INO-4800 immunization to mediate protection of animals against viral challenge.

In summary, the result describing the immunogenicity of COVID-19 vaccine candidate, INO-4800 are promising, and it is particularly encouraging to measure antibody and T cell levels at an early time point after a single dose of the vaccine supporting the further evaluation of this vaccine.

The material and methods used for the experiments are now described

Cell Lines

Human embryonic kidney (HEK)-293T and COS-7 cell lines were obtained from ATCC (Old Town Manassas, Va.). All cell lines were maintained in DMEM supplemented with 10% fetal bovine serum (FBS) and penicillin-streptomycin.

In Vitro RNA Expression (qRT-PCR)

In vitro mRNA expression of the plasmid was demonstrated by transfection of COS-7 with serially diluted plasmids followed by analysis of the total RNA extracted from the cells using reverse transcription and PCR. Transfections of four concentrations of the plasmid were performed using FuGENE® 6 transfection reagent (Promega) which resulted in final masses ranging between 80 and 10 ng per well. The transfections were performed in duplicate. Following 18 to 26 hours of incubation the cells were lysed with RLT Buffer (Qiagen). Total RNA was isolated from each well using the Qiagen RNeasy kit following the kit instructions. The resulting RNA concentration was determined by OD_{260/280} and samples of the RNA were diluted to 10 ng per 4. One hundred nanograms of RNA was then converted to cDNA using the High Capacity cDNA Reverse Transcription (RevT) kit (Applied Biosystems) following the kit instructions. RevT reactions containing RNA but no reverse transcriptase (minus RT) were included as controls for plasmid DNA or cellular genomic DNA sample contamination. Eight μL of sample cDNA were then subjected to PCR using primers and probes that are specific to the target sequence. In a separate reaction, the same quantity of sample cDNA was subjected to PCR using primers and probe designed for COS-7 cell line β-actin sequences. Using a QuantStudio 7 Flex Real Time PCR Studio System (Applied Biosystems), samples were first subjected to a hold of 1 minute at 95° C. and then 40 cycles of PCR with each cycle consisting of 1 second at 95° C. and 20 seconds at 60° C. Following PCR, the amplifications results were analyzed as follows. The negative transfection controls, the minus RevT controls, and the NTC were scrutinized for each of their respective indications. The threshold cycle (C_{T}) of each transfection concentration for the INO-4800 COVID-19 target mRNA and for the β-actin mRNA was generated from the QuantStudio software using an automatic threshold setting. The plasmid was considered to be active for mRNA expression if the expression in any of the plasmid transfected wells compared to the negative transfection controls were greater than 5 C_{T}.

In Vitro Protein Expression (Western Blot)

Human embryonic kidney cells, 293T were cultured and transfected as described previously (Yan et al., 2007, Mol Ther, 15:411-421). 293T cells were transfected with pDNA using TurboFectin8.0 (OriGene) transfection reagent following the manufacturer's protocol. Forty-eight hours later cell lysates were harvested using modified RIPA cell lysis buffer. Proteins were separated on a 4-12% BIS-TRIS gel (ThermoFisher Scientific), then following transfer, blots were incubated with an anti-SARS-CoV spike protein polyclonal antibody (Novus Biologicals) then visualized with horseradish peroxidase (HRP)-conjugated anti-mouse IgG (GE Amersham).

Immunofluorescence of Transfected 293T Cells

For in vitro staining of Spike protein expression 293T cells were cultured on 4-well glass slides (Lab-Tek) and transfected with 3 μg per well of pDNA using TurboFectin8.0 (OriGene) transfection reagent following the manufacturer's protocol. Cells were fixed 48 hrs after transfection with 10% Neutral-buffered Formalin (BBC Biochemical, Washington State) for 10 min at room temperature (RT) and then washed with PBS. Before staining, chamber slides were blocked with 0.3% (v/v) Triton-X (Sigma), 2% (v/v) donkey serum in PBS for 1 hr at RT. Cells were stained with a rabbit anti-SARS-CoV spike protein polyclonal antibody (Novus Biologicals) diluted in 1% (w/v) BSA (Sigma), 2% (v/v) donkey serum, 0.3% (v/v) Triton-X (Sigma) and 0.025% (v/v) 1 g ml⁻¹ Sodium Azide (Sigma) in PBS for 2 hrs at RT. Slides were washed three times for 5 min in PBS and then stained with donkey anti-rabbit IgG AF488 (lifetechnologies) for 1 hr at RT. Slides were washed again and mounted and covered with DAPI-Fluoromount (SouthernBiotech).

Animals

Female, 6 week old C57/BL6 and BALB/c mice were purchased from Charles River Laboratories (Malvern, Pa.) and The Jackson Laboratory (Bar Harbor, Me.). Female, 8 week old Hartley guinea pigs were purchased from Elm Hill Labs (Chelmsford, Mass.). For mouse studies, on day 0 doses of 2.5, 10 or 25 μg pDNA were administered to the tibialis anterior (TA) muscle by needle injection followed by CELLECTRA® in vivo electroporation (EP). The CELLECTRA® EP delivery consists of two sets of pulses with 0.2 Amp constant current. Second pulse sets is delayed 3 seconds. Within each set there are two 52 ms pulses with a 198 ms delay between the pulses. On days 0 and 14 blood was collected. Parallel groups of mice were serially sacrificed on days 4, 7, and 10 post-immunization for analysis of cellular immune responses. For guinea pig studies, on day 0, 100 μg pDNA was administered to the skin by Mantoux injection followed by CELLECTRA® in vivo EP. Blood was collected on day 0 and 14. For ACE2 competition ELISAs and lung bronchoalveolar lavage, mice and guinea pigs were immunized twice at days 0 and 14. Sera was collected 14 days post-2^{nd} immunization for mice and 28 days post-2nd immunization for guinea pigs.

Antigen Binding ELISA

ELISAs were performed to determine sera antibody binding titers. Nunc ELISA plates were coated with 1 μg ml⁻¹ recombinant protein antigens in Dulbecco's phosphate-buffered saline (DPBS) overnight at 4° C. Plates were washed three times then blocked with 3% bovine serum albumin (BSA) in DPBS with 0.05% Tween 20 for 2 hours at 37° C. Plates were then washed and incubated with serial dilutions of mouse or guinea pig sera and incubated for 2 hours at 37° C. Plates were again washed and then incubated with 1:10,000 dilution of horse radish peroxidase (HRP) conjugated anti-guinea pig IgG secondary antibody (Sigma-Aldrich, cat. A7289) or (HRP) conjugated anti-mouse IgG secondary antibody (Sigma-Aldrich) and incubated for 1 hour at RT. After final wash plates were developed using SureBlue™ TMB 1-Component Peroxidase Substrate (KPL, cat. 52-00-03) and the reaction stopped with TMB Stop Solution (KPL, cat. 50-85-06). Plates were read at 450 nm wavelength within 30 minutes using a Synergy HTX (BioTek Instruments, Highland Park, Vt.). Binding antibody endpoint titers (EPTs) were calculated as previously described in Bagarazzi Metal. 2012 (Bagarazzi et al., 2012, Sci Transl Med, 4:155ra138). Binding antigens tested included, SARS-CoV-2 antigens: 51 spike protein (Sino Biological 40591-V08H), S1+S2 ECD spike protein (Sino Biological 40589-V08B1), RBD (University of Texas, at Austin (McLellan Lab.)); SARS-COV antigens: Spike 51 protein (Sino Biological 40150-V08B1), S (1-1190) (Immune Tech IT-002-001P) and Spike C-terminal (Meridian Life Science R18572).

ACE-2 Competition ELISA

Mice

ELISAs were performed to determine sera IgG antibody competition against human ACE2 with a human Fc tag. Nunc ELISA plates were coated with 1 μg/ml rabbit anti-His6× in 1×PBS for 4-6 hours at room temperature and washed 4 times with washing buffer (1×PBS and 0.05% Tween 20). Plates were blocked overnight at 4° C. with blocking buffer (1×PBS, 0.05% Tween 20, 5% evaporated milk and 1% FBS). Plates were washed four times with washing buffer then incubated with full length (S1+S2) spike protein containing a C-terminal His tag (Sino Biologics, cat. 40589-V08B1) at 10 ug/ml for 1 hour at room temperature. Plates were washed and then serial dilutions of purified mouse IgG mixed with 0.1 ug/ml recombinant human ACE2 with a human Fc tag (ACE2-IgHu) were incubated for 1-2 hours at room temperature. Plates were again washed and then incubated with 1:10,000 dilution of horse radish peroxidase (HRP) conjugated anti-human IgG secondary antibody (Bethyl, cat. A80-304P) and incubated for 1 hour at room temperature. After final wash plates were developed using 1-Step Ultra TMB-ELISA Substrate (Thermo, cat. 34029) and the reaction stopped with 1 M Sulfuric Acid. Plates were read at 450 nm wavelength within 30 minutes using a SpectraMax Plus 384 Microplate Reader (Molecular Devices, Sunnyvale, Calif.). Competition curves were plotted and the area under the curve (AUC) was calculated using Prism 8 analysis software with multiple t-tests to determine statistical significance.

Guinea Pigs

96 well half area assay plates (Costar) were coated with 25 μl/well of 5 μg/mL of SARS-CoV-2 spike S1+S2 protein (Sino Biological) diluted in 1×DPBS (Thermofisher) overnight at 4° C. Plates were washed with 1×PBS buffer with 0.05% TWEEN (Sigma). 100 μl/well of 3% (w/v) BSA (Sigma) in 1×PBS with 0.05% TWEEN were added and incubated for 1 hr at 37° C. Serum samples were diluted 1:20 in 1% (w/v) BSA in 1×PBS with 0.05% TWEEN. After washing the assay plate, 25 μl/well of diluted serum was added and incubated 1 hr at 37° C. Human recombinant ACE-2-Fc-tag (Sinobiological) was added directly to the diluted serum, followed by 1 hr of incubation at 37° C. Plates were washed and 25 μl/well of 1:10,000 diluted goat anti-hu Fc fragment antibody HRP (bethyl) was added to the assay plate. Plates were incubated 1 hr at room temperature. For development the SureBlue/TMB Stop Solution (KPL, MD) was used and O.D. was recorded at 450 nm.

Bronchoalveolar Lavage Collection

Bronchoalveolar lavage (BAL) fluid was collected by washing the lungs of euthanized and exsanguinated mice with 700-1000 ul of ice-cold PBS containing 100 μm EDTA, 0.05% sodium azide, 0.05% Tween-20, and 1× protease inhibitor (Pierce) (mucosal prep solutions (MPS) with a blunt-ended needle. Guinea pig lungs were washed with 20 ml of MPS via 16 G catheter inserted into the trachea. Collected BAL fluid was stored at −20 C until the time of assay.

IFN-γ ELISpot

Spleens from mice were collected individually in RPMI1640 media supplemented with 10% FBS (R10) and penicillin/streptomycin and processed into single cell suspensions. Cell pellets were re-suspended in 5 mL of ACK lysis buffer (Life Technologies, Carlsbad, Calif.) for 5 min RT, and PBS was then added to stop the reaction. The samples were again centrifuged at 1,500 g for 10 min, cell pellets re-suspended in R10, and then passed through a 45 μm nylon filter before use in ELISpot assay. ELISpot assays were performed using the Mouse IFN-γ ELISpot^{PLUS} plates (MABTECH). 96-well ELISpot plates pre-coated with capture antibody were blocked with R10 medium overnight at 4° C. 200,000 mouse splenocytes were plated into each well and stimulated for 20 hours with pools of 15-mer peptides overlapping by 9 amino acid from the SARS-CoV-2, SARS-CoV, or MERS-CoV Spike proteins (5 peptide pools per protein). Additionally, matrix mapping was performed using peptide pools in a matrix designed to identify immunodominant responses. Cells were stimulated with a final concentration of 5 μL of each peptide per well in RPMI+10% FBS (R10). The spots were developed based on manufacturer's instructions. R10 and cell stimulation cocktails (Invitrogen) were used for negative and positive controls, respectively. Spots were scanned and quantified by ImmunoSpot CTL reader. Spot-forming unit (SFU) per million cells was calculated by subtracting the negative control wells.

Flow Cytometry

Intracellular cytokine staining was performed on splenocytes harvested from BALB/c and C57BL/6 mice stimulated with the overlapping peptides spanning the SARS-CoV-2 S protein for 6 hours at 37° C., 5% CO₂. Cells were stained with the following antibodies: FITC anti-mouse CD107a, PerCP-Cy5.5 anti-mouse CD4 (BD Biosciences), APC anti-mouse CD8a (BD Biosciences), ViViD Dye (LIVE/DEAD® Fixable Violet Dead Cell Stain kit; Invitrogen, L34955), APC-Cy7 anti-mouse CD3e (BD Biosciences), and BV605 anti-mouse IFN-γ (eBiosciences). Phorbol Myristate Acetate (PMA) were used as a positive control, and complete medium only as the negative control. Cells were washed, fixed and, cell events were acquired using an FACS CANTO (BD Biosciences), followed by FlowJo software (FlowJo LLC, Ashland, Oreg.) analysis.

Structural Modeling

The structural models for SARS-CoV and MERS-CoV were constructed from PDB IDs 6acc and 5×59 in order to assemble a prefusion model with all three RBDs in the down conformation. The SARS-CoV-2 structural model was built by using SARS-CoV structure (PDB id:6acc) as a template. Rosetta remodel simulations were employed to make the appropriate amino acid mutations and to build de novo models for SARS-CoV-2 loops not structurally defined in the SARS-CoV structure (Huang et al., 2011, PLoS One, 6:e24109). Amino acid positions neighboring the loops were allowed to change backbone conformation to accommodate the new loops. The structural figures were made using PyMOL.

Statistics

All statistical analyses were performed using GraphPad Prism 7 or 8 software (La Jolla, Calif.). These data were considered significant if p<0.05. The lines in all graphs represent the mean value and error bars represent the standard deviation. No samples or animals were excluded from the analysis. Randomization was not performed for the animal studies. Samples and animals were not blinded before performing each experiment.

The Experimental Results are Now Described

Design and Synthesis COVID-19 Synthetic DNA Vaccine Constructs

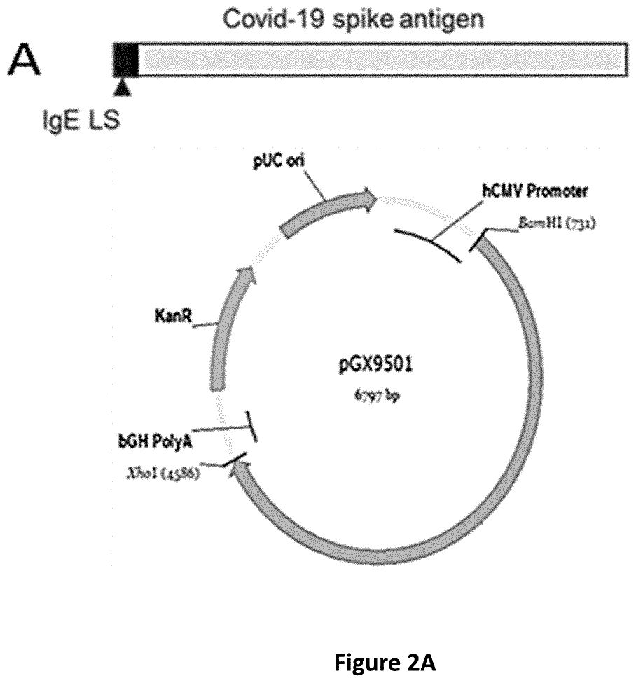

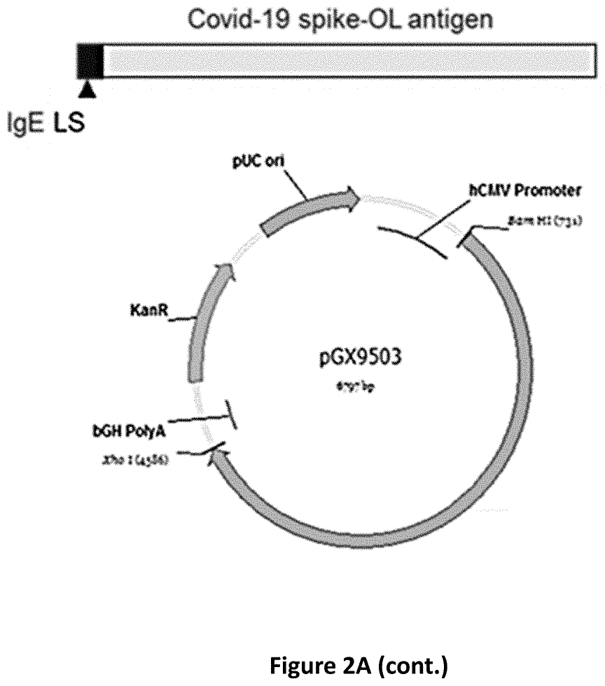

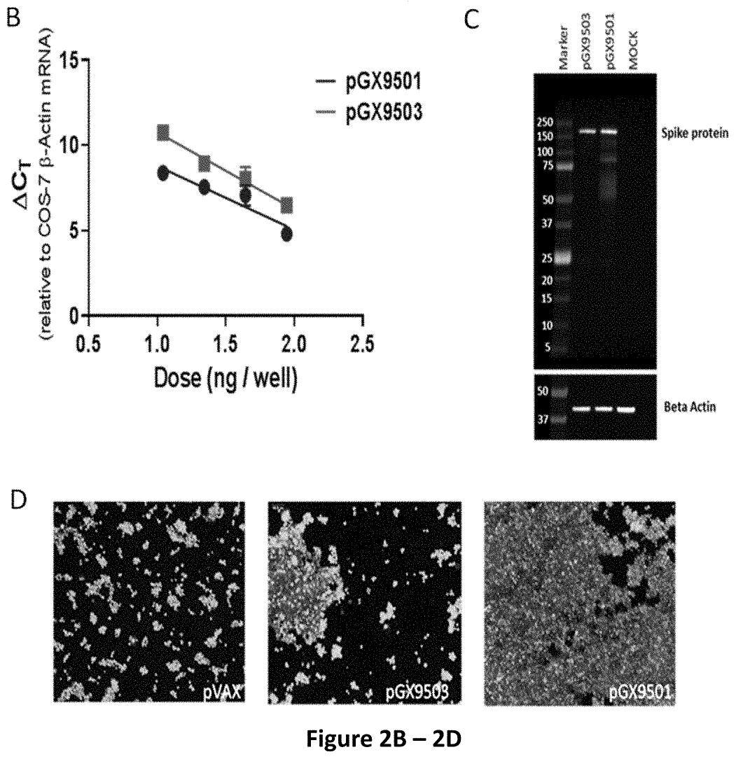

Four spike protein sequences were retrieved from the first four available SARS-CoV-2 full genome sequences published on GISAID (Global Initiative on Sharing All Influenza Data). Three Spike sequences were 100% matched and one was considered an outlier (98.6% sequence identity with the other sequences). After performing a sequence alignment, the SARS-CoV-2 spike glycoprotein sequence was generated and an N-terminal IgE leader sequence was added. The highly optimized DNA sequence encoding SARS-CoV-2 IgE-spike was created using Inovio's proprietary in silico Gene Optimization Algorithm to enhance expression and immunogenicity. The optimized DNA sequence was synthesized, digested with BamHI and XhoI, and cloned into the expression vector pGX0001 under the control of the human cytomegalovirus immediate-early promoter and a bovine growth hormone polyadenylation signal. The resulting plasmids were designated as pGX9501 and pGX9503, designed to encode the SARS-CoV-2 S protein from the 3 matched sequences and the outlier sequence, respectively (FIG. 2A).

In Vitro Characterization of COVID-19 Synthetic DNA Vaccine Constructs

The expression of the encoded SARS-CoV-2 spike transgene was measured at the RNA level in COS-7 cells transfected with pGX9501 and pGX9503. Using the total RNA extracted from the transfected COS-7 cells expression of the spike transgene by RT-PCR was confirmed (FIG. 2B). In vitro spike protein expression in HEK-293T cells was measured by Western blot analysis using a cross-reactive antibody against SARS-CoV S protein on cell lysates. Western blots of the lysates of HEK-293T cells transfected with pGX9501 or pGX9503 constructs revealed bands approximate to the S protein molecular weight of 140-142 kDa (FIG. 2C). In immunofluorescent studies the S protein was detected in 293T cells transfected with pGX9501 or pGX9503 (FIG. 2D). In summary, in vitro studies revealed the expression of the Spike protein at both the RNA and protein level after transfection of cell lines with the candidate vaccine constructs.

Humoral Immune Responses to SARS-CoV-2 S Protein Antigens Measured in Mice Immunized with INO-4800

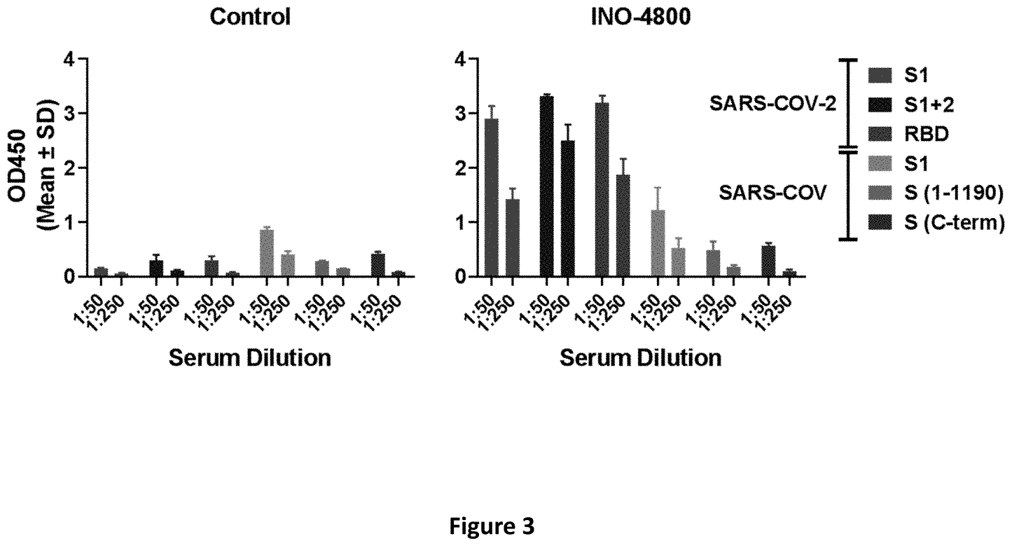

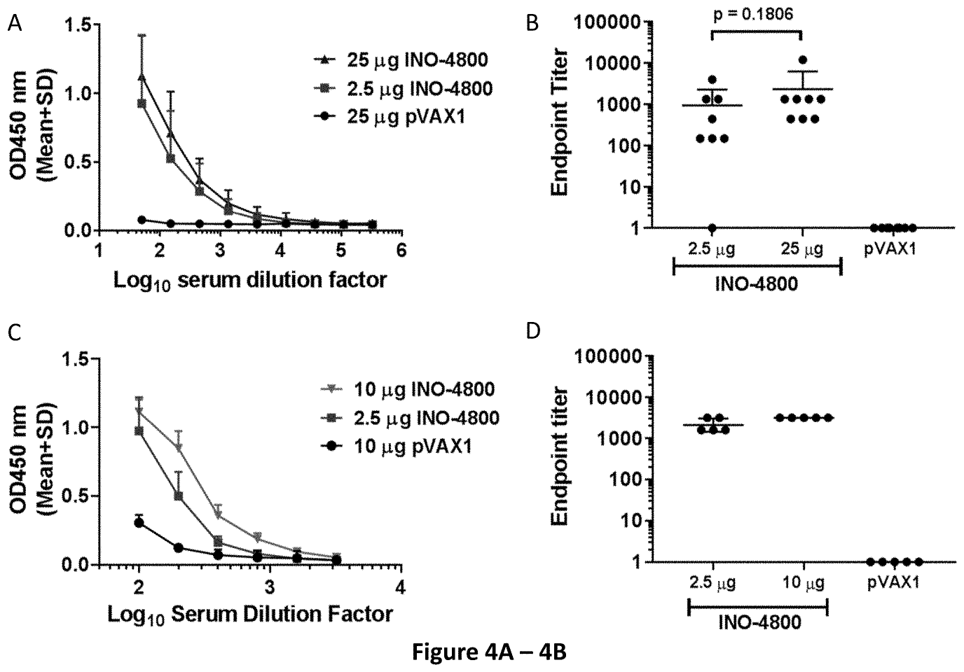

pGX9501 was selected as the vaccine construct to advance to immunogenicity studies, due to the broader coverage it would likely provide compared to the outlier, pGX9503. pGX9501 was subsequently termed INO-4800. The immunogenicity of INO-4800 was evaluated in BALB/c mice, post-administration to the TA muscle followed with CELLECTRA® delivery device. The reactivity of the sera from a group of mice immunized with INO-4800 was measured against a panel of SARS-CoV-2 and SARS-CoV antigens (FIG. 3). Analysis revealed IgG binding against SARS-CoV-2 S protein antigens, with limited cross-reactivity to SARS-CoV S protein antigens, in the serum of INO-4800 immunized mice. The serum IgG binding endpoint titers were measured in mice immunized with pDNA against recombinant SARS-CoV-2 spike protein S1+S2 regions (FIG. 4A and FIG. 4B) and recombinant SARS-CoV-2 spike protein receptor binding domain (RBD) (FIG. 4C and FIG. 4D). Endpoint titers were observed in the serum of mice at day 14 after immunization with a single dose of INO-4800 (FIG. 4B through FIG. 4D).

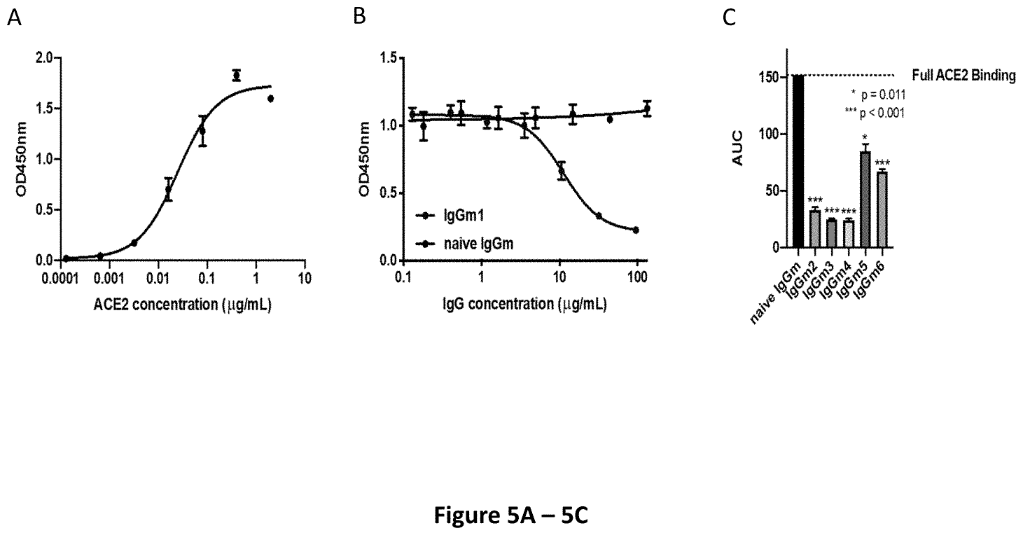

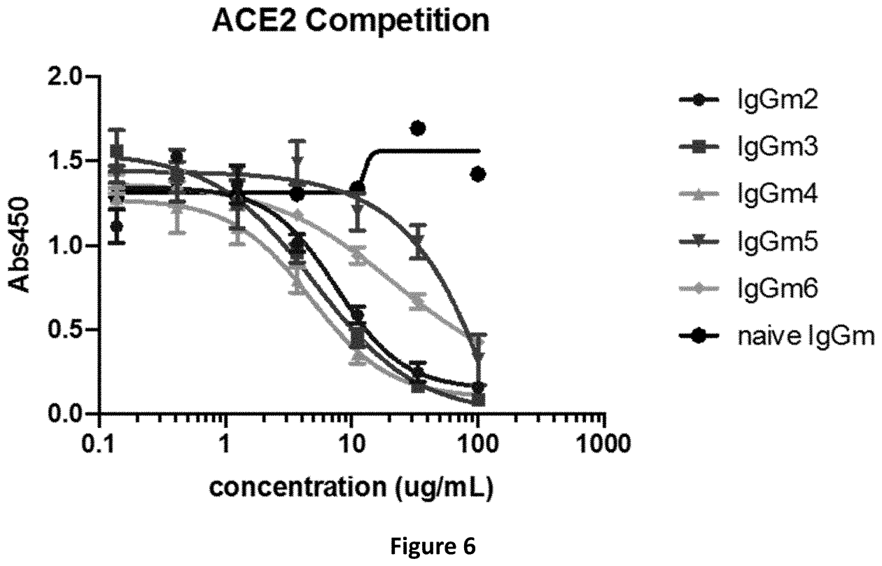

The induction of antibodies capable of inhibiting Spike protein host receptor engagement is a critical goal in SARS-CoV-2 vaccine development. Therefore, experiments were designed to examine the receptor inhibiting functionality of INO-4800-induced antibody responses. Recently an ELISA-based ACE2 inhibition assay was developed as a surrogate for neutralization. The assay is similar in principle to other surrogate neutralization assays which have been validated for coronaviruses (Rosen et al., 2019, J Virol Methods, 265:77-83). As a control in the assay, it was shown that ACE2 can bind to SARS-CoV-2 Spike protein with an EC₅₀ of 0.025 μg/ml (FIG. 5A). BALB/c mice were immunized, on Days 0 and Day 14, with 10 μg of INO-4800, and serum IgG was purified on Day 28 post-immunization to ensure inhibition is antibody mediated. Inhibition of the Spike-ACE2 interaction using serum IgG from a naïve mouse and from an INO-4800 vaccinated mouse were compared (FIG. 5B). The receptor inhibition assay was repeated with a group of five mice immunized, and it was shown that the INO-4800-induced antibodies competed with ACE2 binding to the SARS-CoV-2 Spike protein (FIG. 5C and FIG. 6). ACE2 is considered to be the primary receptor for SARS-CoV-2 cellular entry, blocking this interaction suggests INO-4800-induced antibodies may prevent host infection.

Detection of Humoral Immune Response to SARS-CoV-2 S Protein in Guinea Pigs after Intradermal Delivery of INO-4800

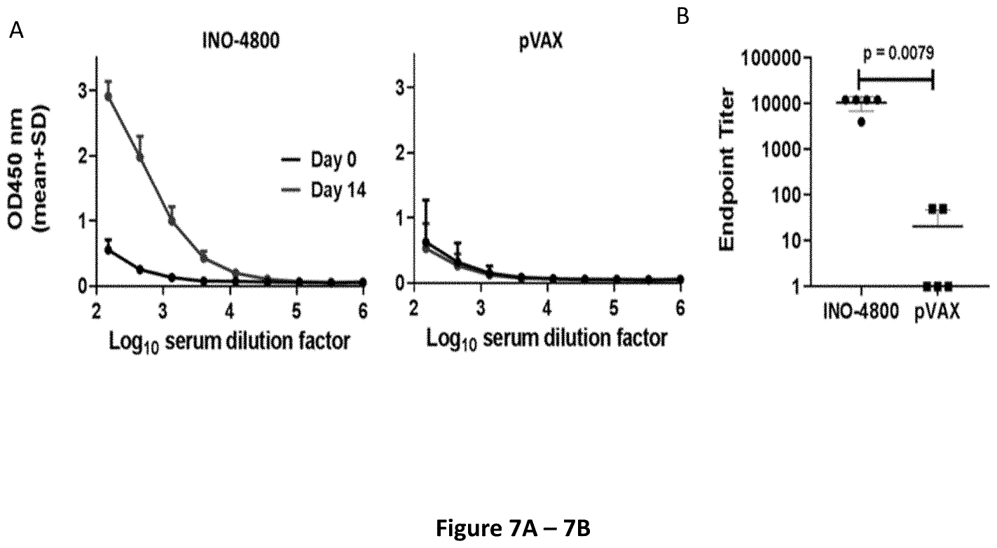

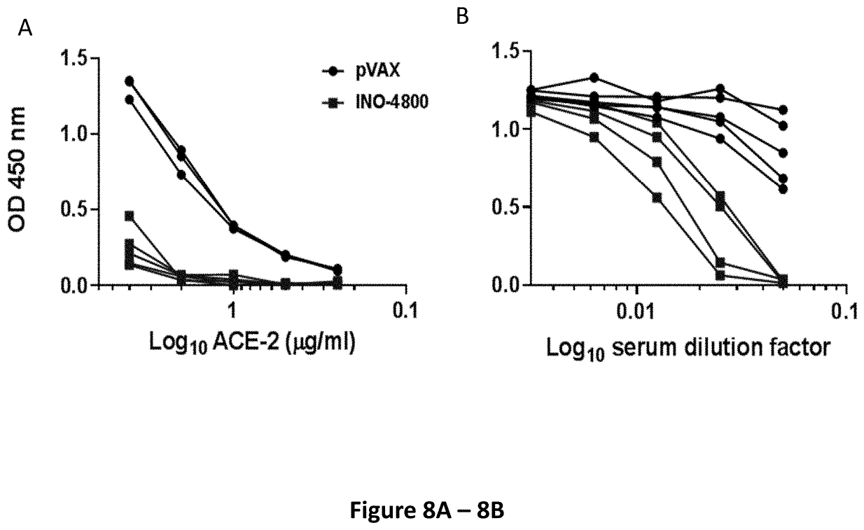

The immunogenicity of INO-4800 was assessed in the Hartley guinea pig model, an established model for intradermal vaccine delivery (Carter et al., 2018, Sci Adv, 4:eaas9930; Schultheis et al., 2017, Vaccine, 35:61-70. 100 μg of pDNA was administered by Mantoux injection to the skin and followed by CELLECTRA® delivery device on day 0 as described in the methods section. On day 14 anti-spike protein binding of serum antibodies was measured by ELISA. Immunization with INO-4800 revealed an immune response in respect to SARS-CoV-2 S1+2 protein binding IgG levels in the serum (FIG. 7A and FIG. 7B). The endpoint SARS-CoV-2 S protein binding titer at day 14 was 10,530 and 21 in guinea pigs treated with 100 μg INO-4800 or pVAX (control), respectively (FIG. 7B). The functionality of the serum antibodies was measured by assessing their ability to inhibit ACE-2 binding to SARS-CoV-2 spike protein. Serum (1:20 dilution) collected from INO-4800 immunized guinea pigs after 2^{nd} immunization inhibited binding of SARS-CoV-2 Spike protein over range of concentrations of ACE-2 (0.25 μg/ml through 4 μg/ml) (FIG. 8A). Furthermore, serum dilution curves revealed serum collected from INO-4800 immunized guinea pigs blocked binding of ACE-2 to SARS-CoV-2 in a dilution-dependent manner (FIG. 8B). Serum collected from pVAX-treated animals displayed negligible activity in the inhibition of ACE-2 binding to the virus protein, the decrease in OD signal at the highest concentration of serum is considered a matrix effect in the assay.

In summary, immunogenicity testing in both mice and guinea pigs revealed the COVID-19 vaccine candidate, INO-4800, was capable of eliciting functional antibody responses to SARS-CoV-2 spike protein.

Biodistribution of SARS-CoV-2 Specific Antibodies to the Lung in INO-4800 Immunized Animals

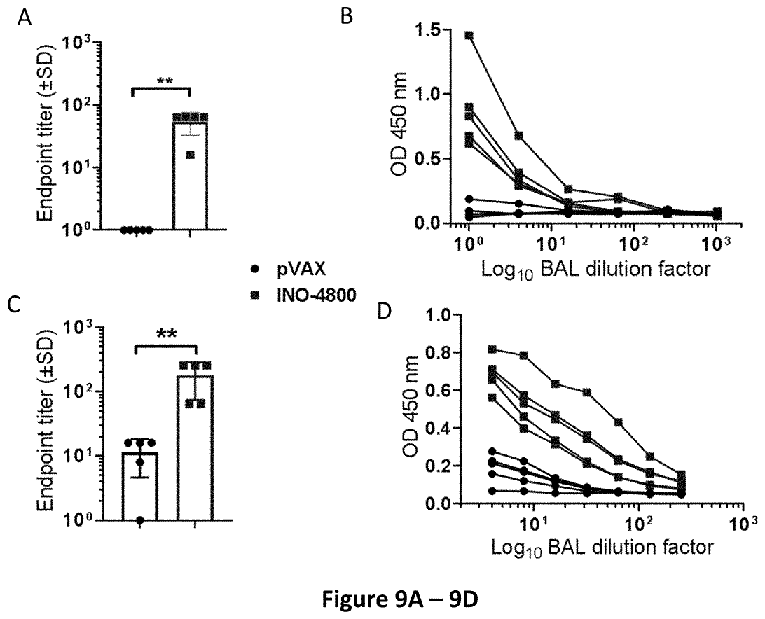

Lower respiratory disease (LRD) is associated with severe cases of COVID-19. The presence of antibodies at the lung mucosa targeting SARS-CoV-2 could potentially mediate protection against LRD. Therefore, the presence of SARS-CoV-2 specific antibody was evaluated in the lungs of immunized mice and guinea pigs. BALB/c mice and Hartley guinea pigs were immunized, on days 0 and 14 or 0, 14 and 28, respectively, with INO-4800 or pVAX control pDNA. Bronchoalveolar lavage (BAL) fluid was collected following sacrifice, and SARS-CoV-2 S protein ELISAs were performed. In both BALB/c and Hartley guinea pigs which received INO-4800 a statistically significant increased SARS-CoV-2 S protein binding IgG was measured in their BAL fluid compared to animals receiving pVAX control (FIG. 9A through FIG. 9D). Taken together, these data demonstrate the presence of anti-SARS-CoV-2 specific antibody in the lungs following immunization with INO-4800.

Early Detection of Cross-Reactive Cellular Immune Responses Against SARS-CoV-2 and SARS-CoV in Mice Immunized with INO-4800

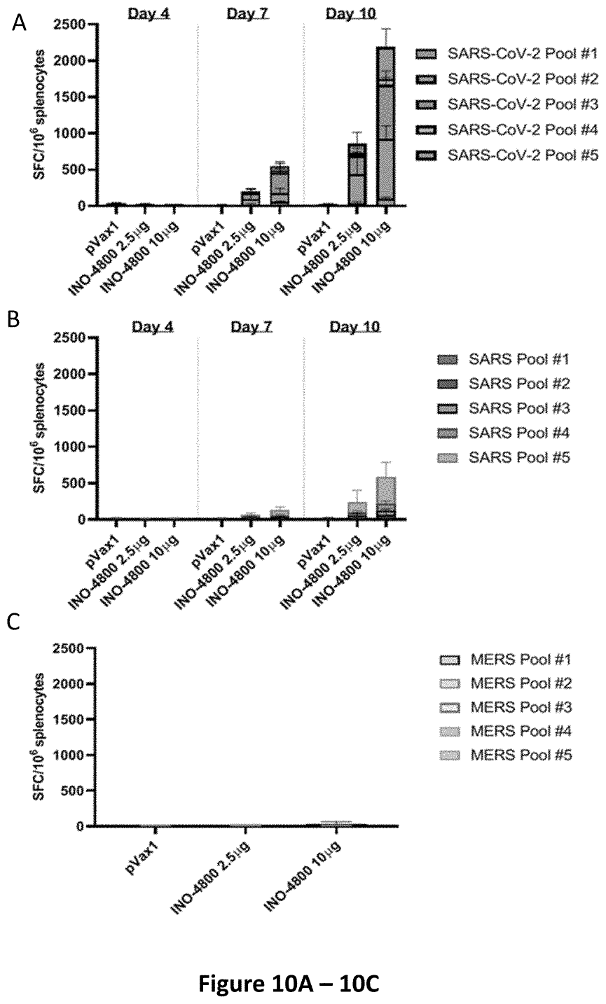

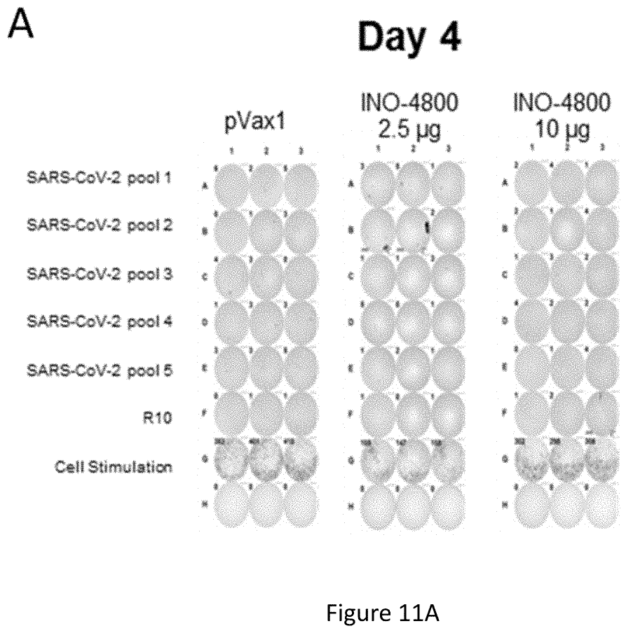

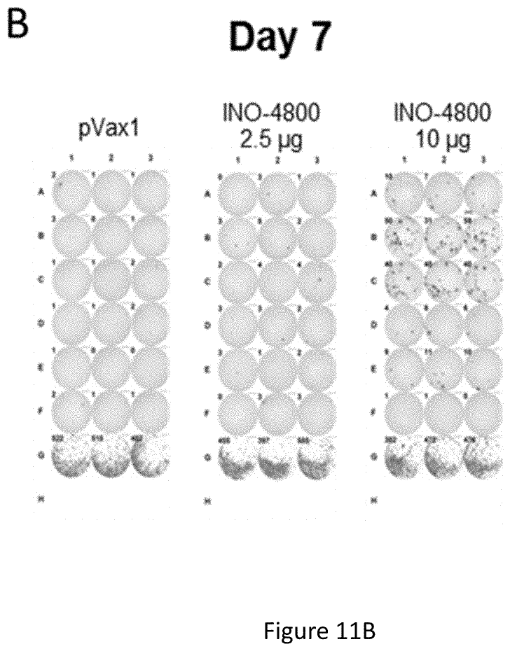

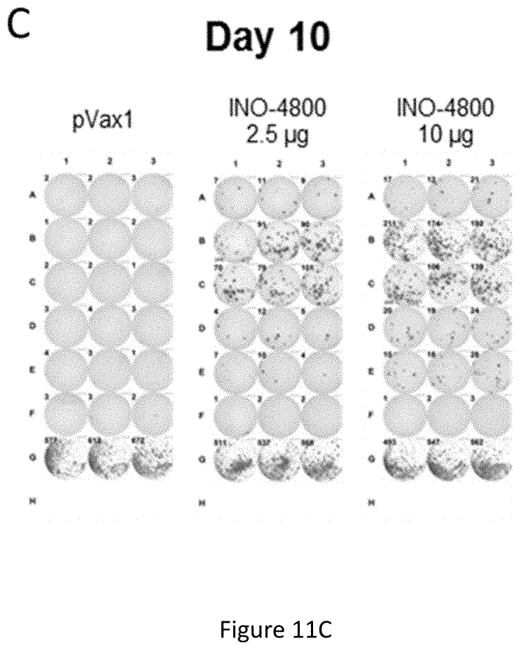

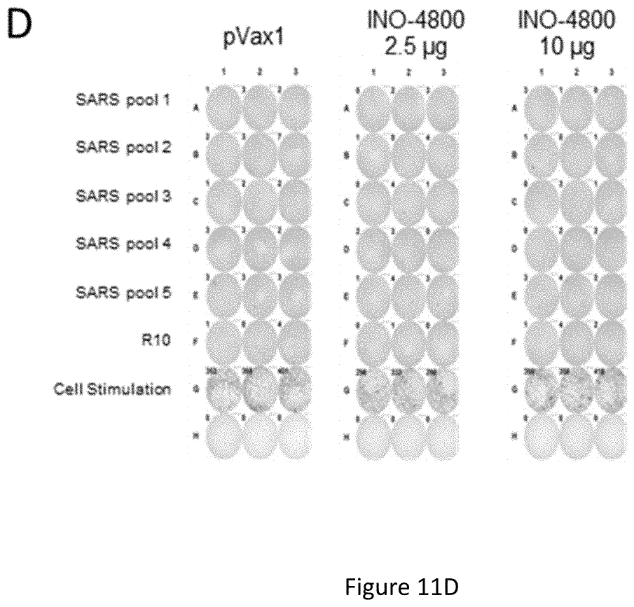

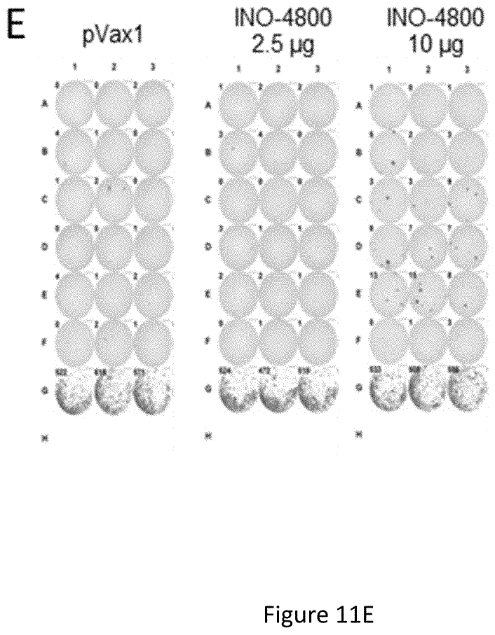

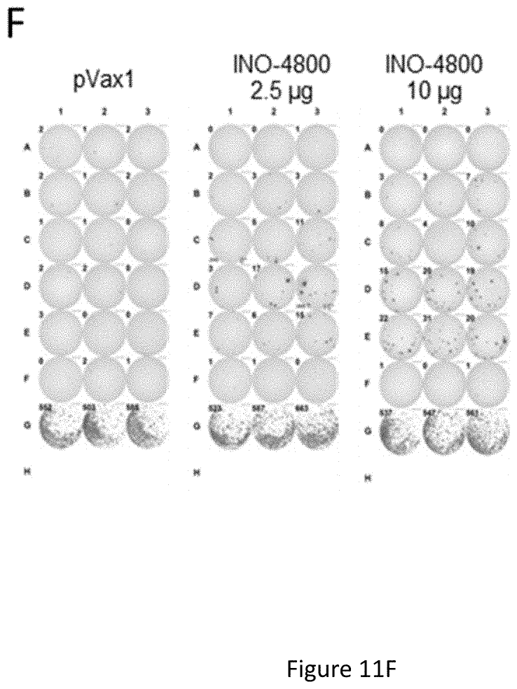

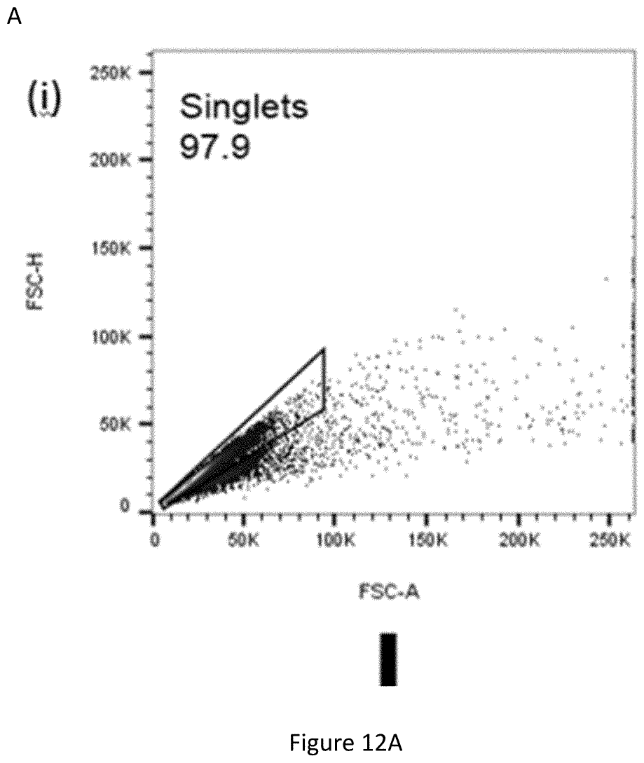

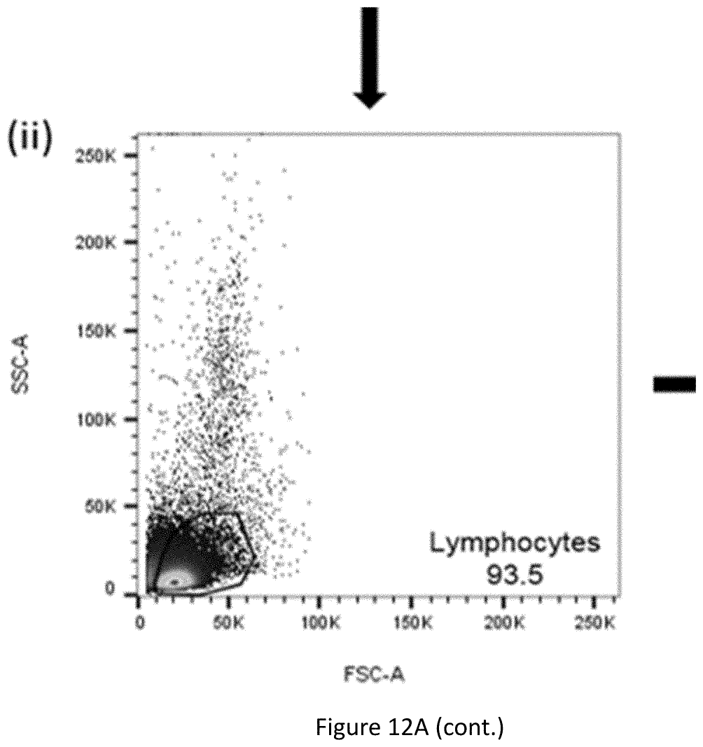

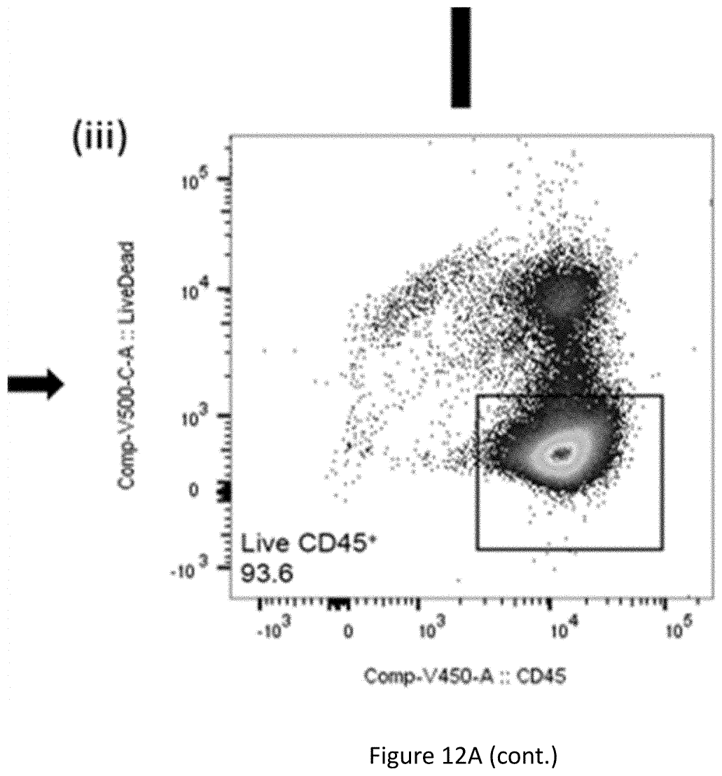

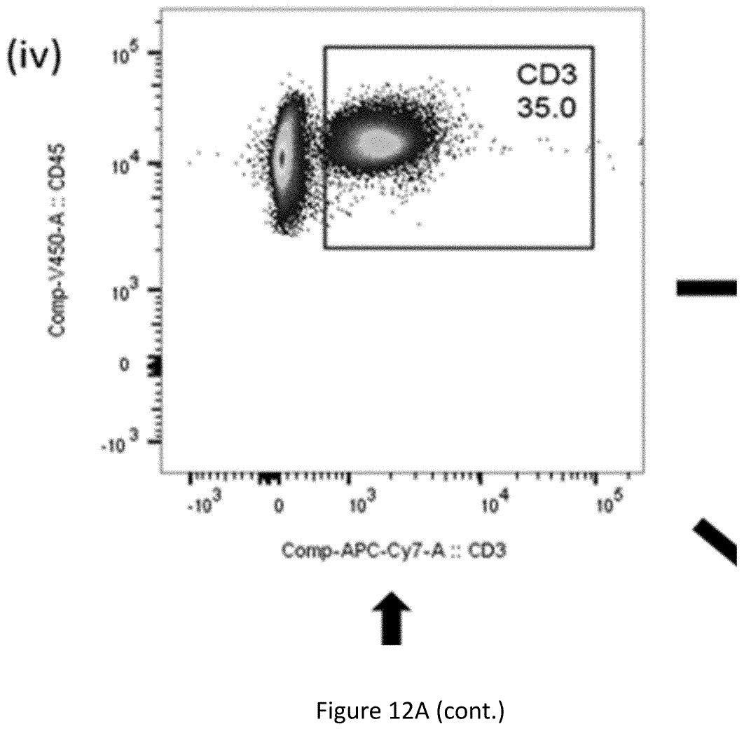

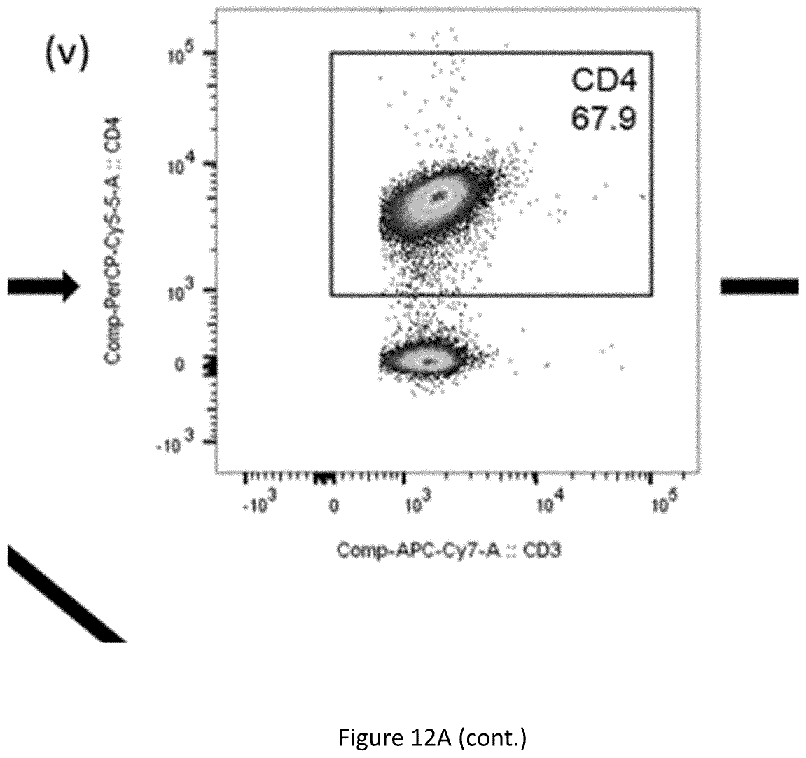

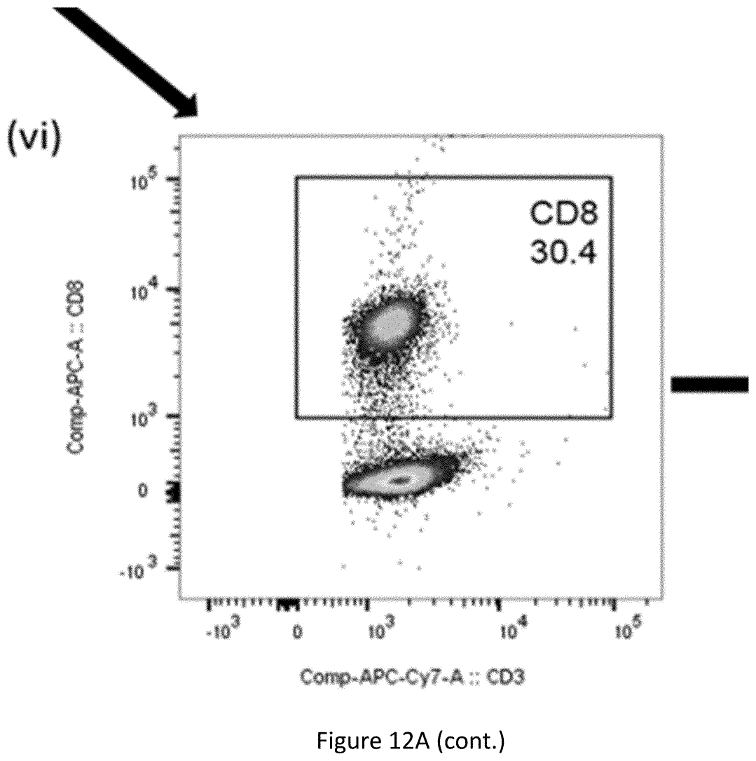

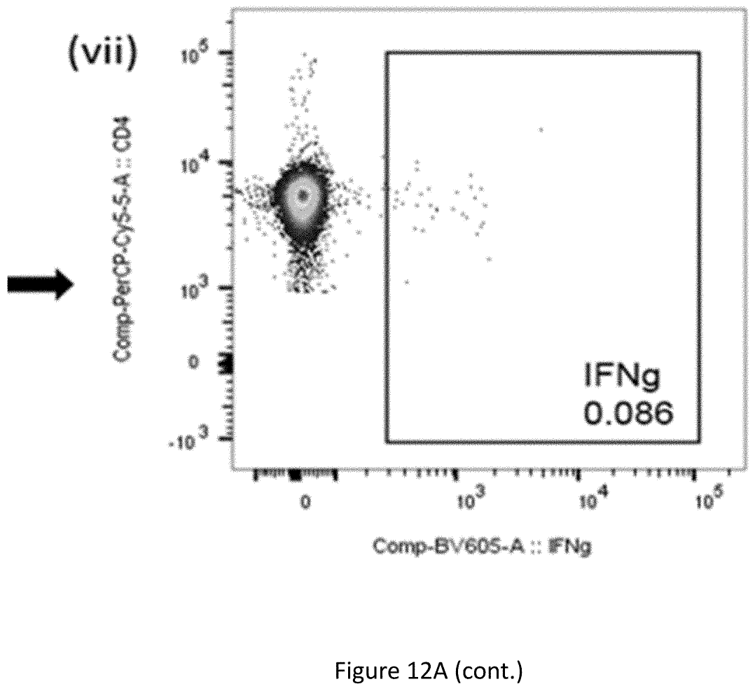

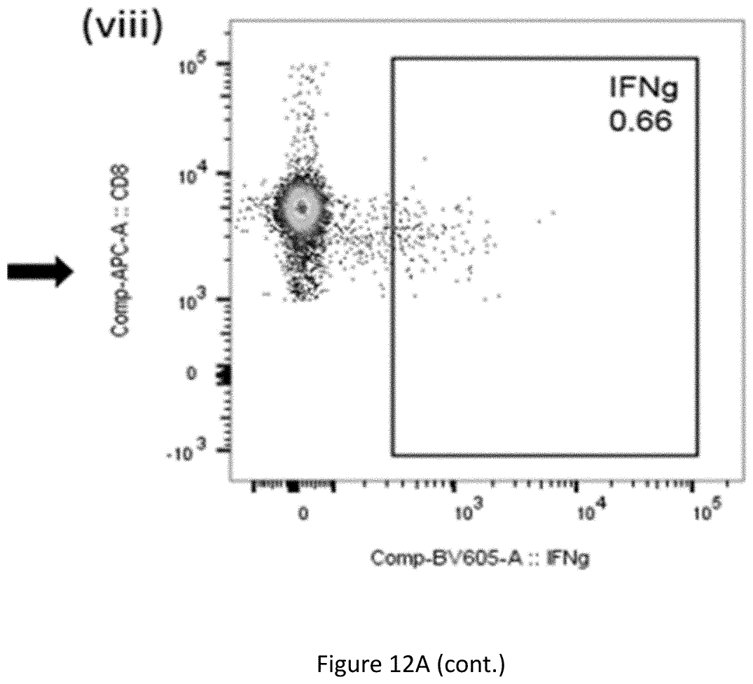

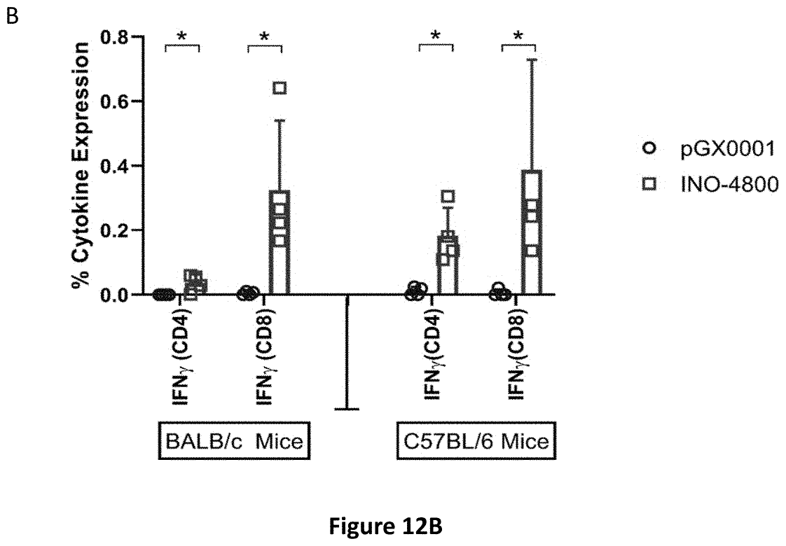

T cell responses against SARS-CoV-2, SARS-CoV, and MERS-CoV S antigens were assayed by IFN-γ ELISpot. Groups of BALB/c mice were sacrificed at days 4, 7, or 10 post-INO-4800 administration (2.5 or 10 μg of pDNA), splenocytes were harvested, and a single-cell suspension was stimulated for 20 hours with pools of 15-mer overlapping peptides spanning the SARS-CoV-2, SARS-CoV, and MERS-CoV spike protein. Day 7 post-INO-4800 administration, T cell responses were measured of 205 and 552 SFU per 10⁶ splenocytes against SARS-CoV-2 for the 2.5 and 10 μg doses, respectively (FIG. 10A). Higher magnitude responses of 852 and 2193 SFU per 10⁶ splenocytes against SARS-CoV-2 were observed on Day 10 post-INO-4800 administration. Additionally, the cross-reactivity of the cellular response elicited by INO-4800 was evaluated against SARS-CoV, and detectable, albeit lower, T cell responses were observed on both Day 7 (74 [2.5 μg dose] and 140 [10 μg dose] SFU per 10⁶ splenocytes) and Day 10 post-administration (242 [2.5 μg dose] and 588 [10 μg dose] SFC per 10⁶ splenocytes) (FIG. 10B). Interestingly, no cross-reactive T cell responses were observed against MERS-CoV peptides (FIG. 10C). Representative images of the IFN-γ ELISpot plates are provided in FIG. 11. The T cell populations which were producing IFN-γ were identified. Flow cytometric analysis on splenocytes harvested from BALB/c mice on Day 14 after a single INO-4800 immunization revealed the T cell compartment to contain 0.0365% CD4+ and 0.3248% CD8+ IFN-γ+ T cells after stimulation with SARS-CoV-2 antigens (FIG. 12).

BALB/c Mouse SARS-CoV-2 Epitope Mapping

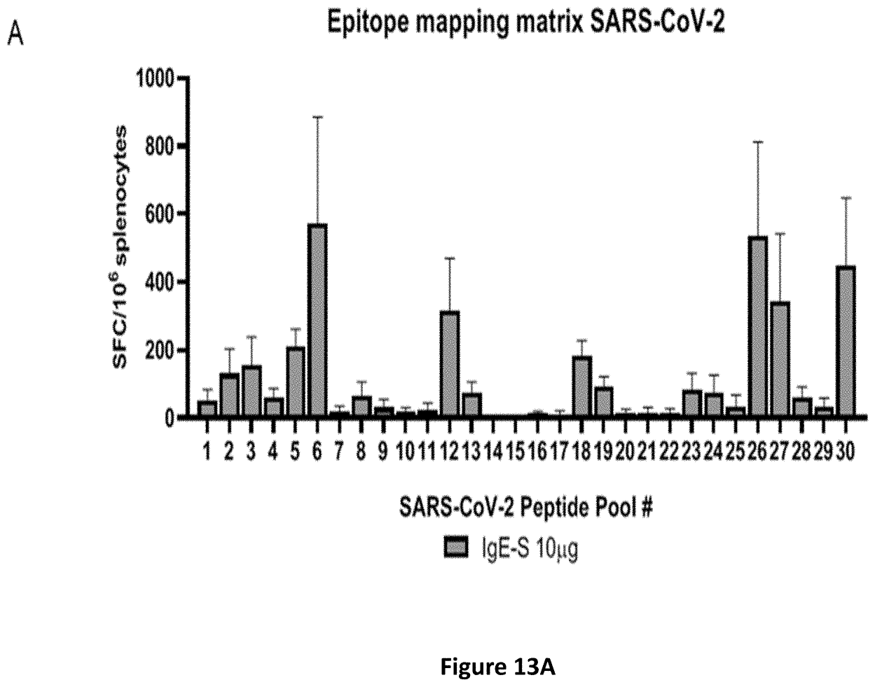

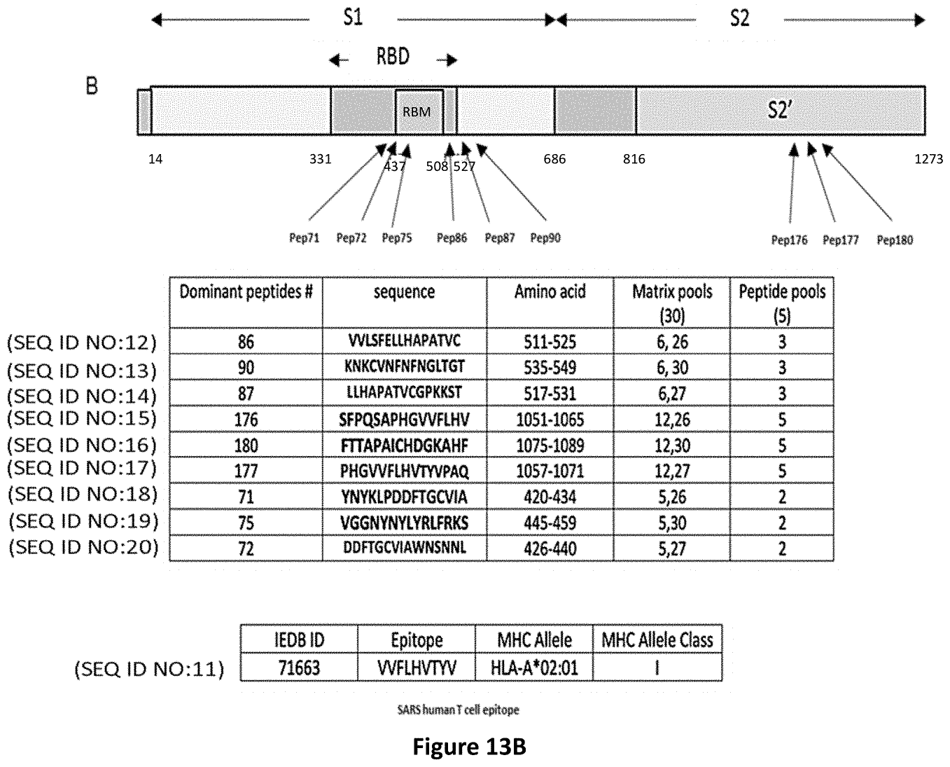

Epitope mapping was performed on the splenocytes from BALB/c mice receiving the 10 μg INO-4800 dose. Thirty matrix mapping pools were used to stimulate splenocytes for 20 hours and immunodominant responses were detected in multiple peptide pools (FIG. 13A). The responses were deconvoluted to identify several epitopes (H2-K^{d}) clustering in the receptor binding domain and in the S2 domain (FIG. 13B). Interestingly, one SARS-CoV-2 H2-K^{d} epitope, PHGVVFLHV (SEQ ID NO:10), was observed to be overlapping and adjacent to the SARS-CoV human HLA-A2 restricted epitope VVFLHVTYV (SEQ ID NO:11) (Ahmed et al., 2020, Viruses 12:254).

In summary, rapid T cell responses against SARS-CoV-2 S protein epitopes were detected in mice immunized with INO-4800.

### Example 2

### Advanced DNA Immunotherapy for Postexposure Protection Against COVID-19

SARS-CoV-2 is the causative agent in COVID-19 that is rapidly developing into a global pandemic. As of May 5, 2020, there are 3,610,006 confirmed cases and 252,346 deaths related to COVID-19 worldwide. COVID-19 represents a direct health threat to the world population, including warfighters, with hundreds of infected U.S. Navy Sailors and infections reported on U.S. Bases, with thousands at-risk. For example, as of May 4, 2020, there are 4,912 cumulative cases in the U.S. military, including 99 hospitalizations and 2 deaths due to COVID-19. Rapid medical countermeasures are urgently needed to allow for more rapid recoveries as well as limiting SARS-CoV-2/COVID-19 transmission in military facilities.

Once infection occurs, the clinical course is variable. Current data suggests that fewer than 2.5% of infected persons show symptoms within 2.2 days (CI, 1.8 to 2.9 days) of exposure, and symptom onset occurs within 11.5 days (CI, 8.2 to 15.6 days) for 97.5% of infected persons. In most (˜80%) cases, COVID-19 presents as a mild-to-moderately severe, self-limited acute respiratory illness with fever, cough, and shortness of breath. It remains unclear exactly what the rate of progression of COVID-19 is and what the predictors are for complications, including pneumonia, acute respiratory distress syndrome (ARDS), kidney failure, and death. Older age, male sex, and comorbidities, including diabetes and hypertension, increase the risk for worse outcomes. Severe COVID-19 disease is characterized by a hyper-inflammatory response and is followed by development of acute respiratory distress syndrome that can require mechanical ventilation. Mortality increased dramatically in the elderly, approximately 15%, however infection did not discriminate with age or physical fitness, therefore all populations are considered at-risk, and a high proportion of younger persons go on to develop severe disease.

Overall, approximately about 16% of COVID-19 diagnosed patients progressed to severe disease. Those that progressed, first posed an increased risk in their community before they moved to require treatment in the ICU. They may be considered super-spreaders who are a risk to many in the ICU and if they require ventilation and have the potential to significantly expose health care workers and other patients. Many intubated patients after recovery can have a long recovery period that limit their productivity and return to active lifestyle. The current data indicated that patients who progressed appear to not develop strong antiviral immunity compared to persons who self-controlled infection. Importantly, high neutralizing antibodies alone can be associated with disease progression.

Cytotoxic T lymphocyte (CTL) are inversely associated with disease progression and is associated with earlier recovery in MERS patients (Zhao et al., 2017, Sci Immunol, 2:eaan5393). These observations indicated that a post-diagnosis therapeutic protocol is likely to be highly beneficial to slow and reverse the course of disease by providing rapid antibody and CTL induction. Importantly, as DNA immunogens are not susceptible neutralization as they are produced inside cells away from serum effects, the present example demonstrated that a combined delivery can deliver antibodies and T cells rapidly.

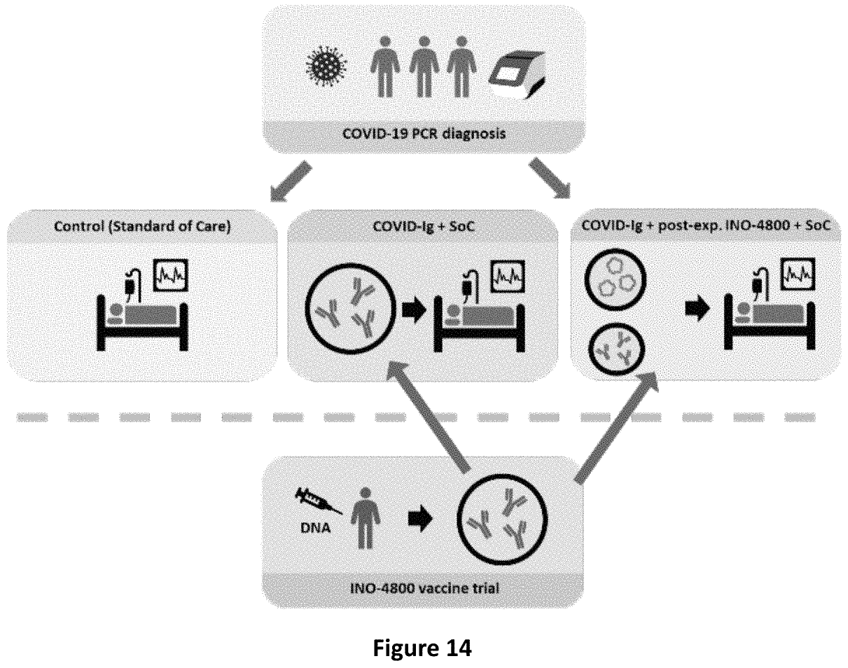

The present example also relates, in part, to the delivery of a combination therapeutic countermeasure built around the development of the SARS-CoV-2 DNA immunogen (INO-4800). This immunotherapy/immunization, named pAI-4800, delivered post-exposure treatment following COVID-19 diagnosis (FIG. 14).

This example also provides significant data demonstrating the safety and efficacy of the herein-described immunotherapy as clinical products for SARS-CoV-2 (INO-4800) as well as MERS have been developed, and describes developed immunotherapeutic approaches for human papillomavirus (HPV) neoplasia studies (NCT01304524, NCT02172911, NCT03185013, NCT03721978), head and neck cancer studies (NCT02163057), glioblastoma studies (NCT03491683), as well as other approaches in this area. The development of a synthetic COVID-19 DNA vaccine was accelerated and moved forward (NCT04336410).

The synthetic DNA vaccine was composed of a full-length the SARS-CoV-2 spike antigen (INO-4800) and delivered intradermally with CELLECTRA smart delivery system. Robust collection of induced antibody data in mice, guinea pigs, rabbits, and non-human primates was generated and published in revision on this immunogen. The DNA immunotherapy drug product was already manufactured, including fill/finish, and CMC has been performed to support initial studies. Based on the strength of herein-described preclinical data, the COVID-19 DNA vaccine pre-exposure prophylaxis safety and immunogenicity study was developed and approved in 10 weeks. The study was fully enrolled in just 20 days. The trial enrolled 40 participants initially and is expanding adding additional subgroups. Current studies are focused on moving into efficacy trials with this approach in thousands of subjects.

pAI-4800 Countermeasure

The herein-described countermeasure is a combination immunotherapy that delivered immunoglobulin (Ig) from healthy volunteers who have previously received INO-4800 as a preferred source of plasma/immunoglobulin and therapeutic immunization with INO-4800.

Part I: Vaccinated persons were easily validated for immune responses and a battery of serology assays was established for selecting potent responders to the DNA vaccine, who developed high neutralizing antibody titer, as the donors for plasmapheresis and the manufacturing of immunoglobulin. The use of vaccine responders as potential donors of plasma offered several advantages: (1) safety—the source of sera for transfer was not potentially contaminated with residual virus from low level PCR positive convalescent individuals; and (2) seropositive after immunization can donate multiple units of plasma, as they can potentially be revaccinated if the antibody titers decrease below the levels that make them susceptible to infection.

For the present study, the COVID intravenous immunoglobulin (COVID-IVIG) is purified and manufactured. The IVIG product that is used in this study is manufactured according to Good Manufacturing Practices (GMP) from human plasma collected under IND at sites that are FDA-registered or licensed to perform this function. Plasma used to manufacture this IVIG product meet the following criteria prior to being used for manufacture: negative Anti-HIV 1/2, negative Anti-HTLV 1/2, negative Anti-HCV, negative HBsAg, negative serologic test for syphilis, negative HIV NAT, negative HCV NAT, negative WNV NAT, negative Hepatitis A virus NAT, negative Hepatitis B virus NAT, and negative Parvovirus B19 NAT. The IVIG that are used in this study have the average SARS-CoV-2 titers of >1:320. The administered immunoglobulin provides an immediate therapeutic benefit potentially neutralizing the initial inoculum and preventing or decrease the risk of developing symptomatic disease.

Part II: The second component of this countermeasure study was to therapeutically immunize the COVID-19 confirmed patients at the time of immune sera transfer. Here, INO-4800 (delivered intradermally with CELLECTRA smart delivery system) is used to drive immune therapeutic T cell as well as expand permanent immunogen driven humoral responses. DNA vaccines were potent inducers of CD8+ T cell responses that were directed to the site of infection. T cells responses correlated with better patient outcome against multiple viruses, including MERS-CoV, a related betacornavirus, and were believed to be important for clearance of SARS and likely for SARS-CoV-2.

The vaccine component also boosts the immune responses induced by the SARS CoV-2 infection and provides long term therapeutic benefits and enhanced durability of immune protection. The study described-herein directly addresses the need for an immune countermeasure to treat COVID-19 for subjects at risk of the disease, including military personnel, health-care workers, and other at-risk or vulnerable populations, such as the elderly or diabetic populations, among others. The immunotherapy described-herein treats infection of military personnel among others and mitigates disease progression.

The herein-described immunotherapy studies rapidly initiate a Phase I study of the post-diagnosis countermeasure, pAI-4800, against COVID-19 and then seeks to expand this study to a Phase II efficacy trial in 2 months. The candidates are advanced into the clinic in less than 3 months using the safety data accrued in the prophylactic human vaccine study.

The scope of the herein-described studies is to prevent the development of advanced COVID-19 disease in the infected individuals, such as at-risk personnel. The objective was to develop a post-exposure immunotherapy that can be rapidly deployed to prevent SARS-CoV-2 infection progression to severe COVID-19 disease. The herein-described study demonstrates the development of a therapy against SARS-CoV-2 infection, or COVID-19.

### Technical Approach

The goal of this study is to develop a post-exposure immunotherapy that can be delivered against SARS-CoV-2 to prevent SARS-CoV-2 transmission in individuals, such as military personnel.

A COVID-19 DNA vaccine, INO-4800, was advanced to the clinic. The trial was allowed to proceed just 10 weeks from project initiation. The safety and immunogenicity of a pre-exposure prophylactic COVID-19 DNA vaccine candidate are being evaluated. In preclinical data, the rapid induction of antibody and cellular immune responses was observed in multiple studies including mice, rabbits, guinea pigs and in nonhuman primates. Additional supportive data was generated from studies targeting another related coronavirus MERS. A countermeasure for MERS (INO-4700) was advanced and it was demonstrated that this vaccine protected NHP from high dose challenge (Muthumani et al., 2015, Sci Transl Med, 7:301ra132). The MERS vaccine induced robust antibody responses, and importantly T cell immunity (Modjarrad et al, 2019, Lancet Infectious Disease, 19: P1013-1022). The antibody responses were similar to those induced in convalescent patient sera, and the T cells induced were greater than those in convalescent patients (Zhao et al., 2017, Sci Immunol, 2:eaan5393). As T cells, are a possible correlate for recovery from MERS-CoV infection, these data indicated applying an immunotherapeutic approach for COVID-19 treatment. Moreover, post-infection administration of INO-4800 rapidly induces protective cellular immune responses as well as humoral responses to reduce disease severity in these infected persons. These immune responses allow infected patients to exhibit immune control faster, lowering the number of patients who progress to severe disease and lowering their viral load and thus limiting transmission of SARS-CoV-2 to other persons including nosocomial transmission. These two outcomes help with the readiness of individuals, such as military personnel, allowing them to return to their posts sooner and impacting (lowering the risk) transmission to other personnel during their therapy and quarantine.

A novel preventative countermeasure vaccine INO-4800 was advanced into the clinic and was in a unique position to rapidly test a unique regime and use of this product as a post-COVID-19 diagnosis therapeutic vaccine to prevent the development of severe COVID disease. The herein-described study is divided into three sections, designed to evaluate and refine this post-exposure immunotherapy platform. In section 1, Phase I study is initiated to evaluate post-exposure immunotherapeutic delivery of passively transferred COVID-specific IgG along with postexposure DNA vaccine delivery as therapy in COVID-19 diagnosed patients. In section 2, regimen optimization studies are performed in non-human primates (NHPs) to further refine the delivery. The human study is rapidly expanded to Phase IIb to further evaluate efficacy of this platform.

### Section 1: Post-Exposure Immunotherapy Phase I Study

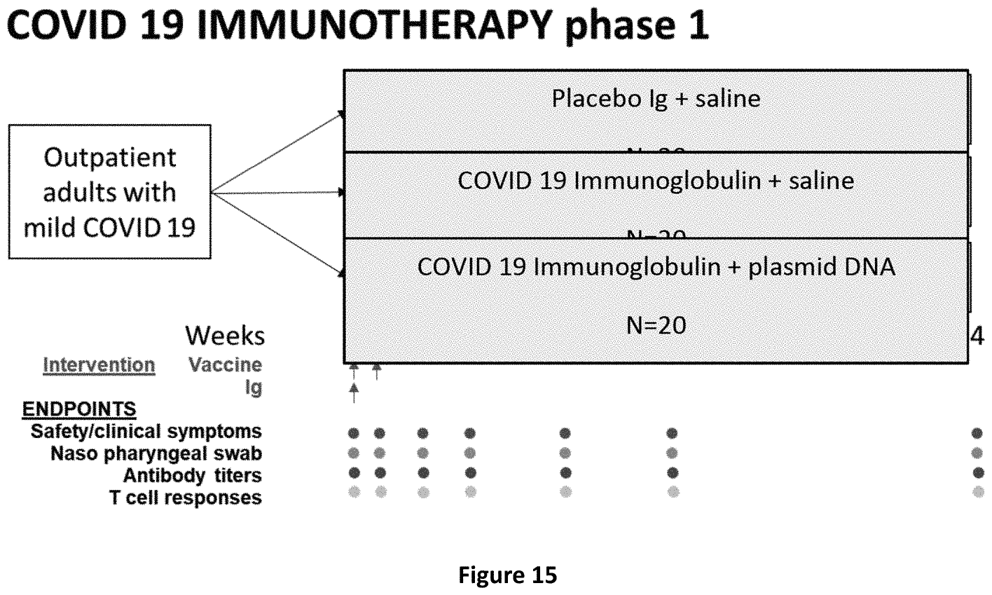

The herein-described study discloses a human clinical study in −60 participants with early COVID-19 symptoms, and PCR positive diagnosis: (1) placebo Ig+placebo plasmid DNA; (2) COVID-19 Ig+placebo plasmid DNA; (3) COVID-Ig+INO-4800 (FIG. 15). All three sections receive current standard-of-care in additional to treatment. The primary endpoint is a prevention of severe COVID-19 disease. Envisioned analytical and research assays include detection of antibody titers, T cell responses (ELISpot assays, flow cytometry, T cell effector assays), cytokine responses, and in vitro function assays (virus neutralization, ACE2 blocking, RBD binding antibodies and flow cytometry-based inhibition assays).

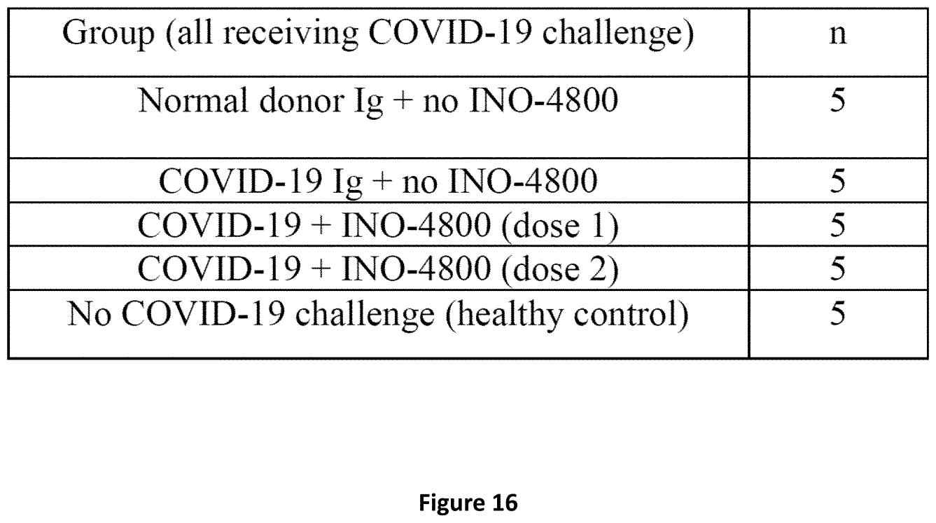

Immunotherapy Criteria: In this double blinded study, patients are diagnosed and then assigned to either 1) standard of care; 2) standard of care+passive immunoglobulin; or 3) immunoglobulin+vaccine (FIG. 16). In the passive immunotherapy arm, patients receive immunotherapy at time of diagnosis. In the vaccine arm, these patients are vaccinated on day 0 and again on day 1. Patients are monitored by standard procedures for progression or recovery.

All patients are bled at day 0, and day 7, and again at day 14, week 4, 8, 12 and 24 to follow immune responses, viral shedding, and durability of the immunological responses. Rigorous immunological assays are performed to follow outcome. On average 4 progressions per arm are likely based on descriptions in the literature and previous experience at the Hospital of the University of Pennsylvania (HUP). Safety are the primary endpoint, with clinical resolution of symptoms, immunogenicity the secondary endpoints. In addition, nasopharyngeal or saliva viral shedding are followed.

### Clinical Diagnostic Assays

Analytical assays include qPCR detection of SARS-CoV-2 virus in nasopharyngeal swabs or saliva following the intervention. Chest x-rays are performed to monitor lung pathology in progressors and in participants that require hospital admission.

### Clinical Research Assays

In parallel, research assays are performed as additionally confirmation assays and to investigate the mode of action of the herein-described immunotherapeutic platform. Additionally,
(1) antibody titers to the SARS-CoV-2 Spike and RBD proteins detected post-immunoglobulin transfer and vaccination with pAI-4800 and
(2) human angiotensin-converting enzyme (ACE2) inhibition assays by surface plasmon resonance (SPR),
(3) ACE2 blocking assays by flow cytometry,
(4) pseudotype neutralization assay,
(5) ELISpot,
(6) flow cytometry detection of cellular immune responses,
(7) cytokine assays are performed by MesoScaleDiscovery (MSD) assays, and
(8) gene expression profiling.

Blood chemistries are also followed for immune cell populations and NK cell function are monitored. Together, these assays are informative to understand the levels of immunity that can be induced in post-exposure vaccinated patients and the correlation of the development of their immune responses temporally to impact on viral shedding. Ultimately the study follows patients towards clinical outcome.

The initial INO-4800 Phase I study received FDA approval and is open as of Apr. 6, 2020. This novel vaccine immunotherapy advances to evaluate the use of post-infection vaccination for mitigation of serious or immediately-life threatening COVID-19 and/or SARS-CoV-2 infections.

It is likely that up to 20% of each cohort might progress to severe disease providing up to 6 persons per arm. While a further, more powered study, are needed to see an impact on disease progression, the efficacy of this approach should be 50% or greater in mitigating progression to severe disease. This provides an important supportive collection of clinical data for a larger efficacy study. If the efficacy is 75% or greater, it should be possible to be observed in this study. In addition, the immune outcomes provide additional supportive data for the ability of such a therapeutic approach to impact viral load. These are exceptional outcomes for this small focused study that are of benefit to the populations.

### Section 2: Regimen Optimization Studies in NHPs

In parallel, regimen optimization studies are performed in a rhesus macaque model (n=25 NHPs, Bioqual, Rockville, Md.). These studies parallel the clinical study to provide additional supportive data for the pAI-4800 platform (FIG. 16).

### Research Assays

For consistency, research assays similar to the clinical assays outlined in Section 1 are performed. These include diagnostic confirmation and analytical assays (qPCR detection of viral load, x-rays, complete blood chemistry analysis) and research assays, including antibody titers, inhibition assays, flow cytometry, neutralization assays, cytokine assays, and additional assay development to support study expansion.

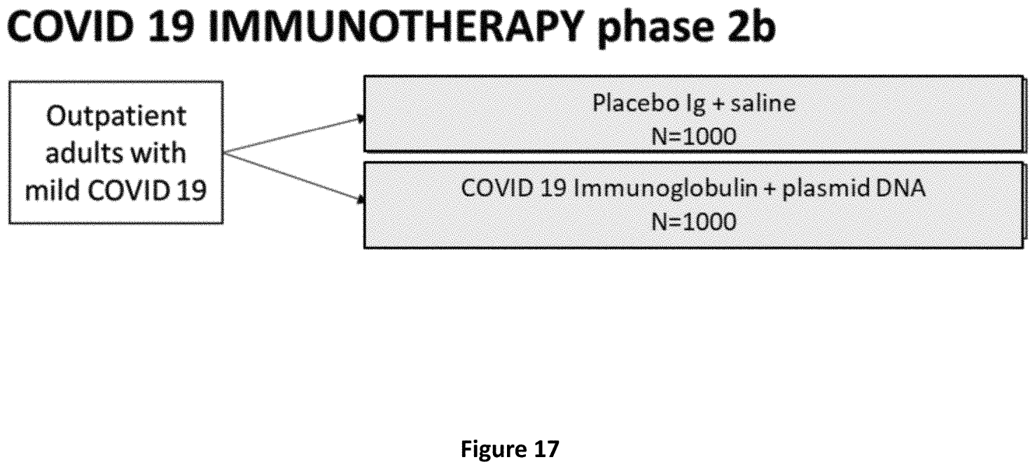

Clinical study expansion: Phase IIb. The results of these studies, in combination with the data from the human phase I study are utilized to support further clinical phase study expansion (FIG. 17). The study was designed to evaluate the efficacy of COVID 19 Immunoglobulin plus INO-4800 vaccination to prevent hospitalizations and death in outpatient adults diagnosed with COVID-19 compared to those receiving placebo. The primary analysis includes testing the equality of proportions hospitalized/dying in the two randomized arms, and construction of 95% confidence intervals (CIs) for the ratio of proportions between the study arms. The primary analysis focuses on comparing the ratio of proportions because of the uncertainty in knowing what the hospitalization/death rate is.

The SARS-CoV-2 genome was made publicly available on Jan. 11, 2020 and the design and development of a COVID-19 DNA vaccine based on the surface Spike glycoprotein was immediately initiated. Animal studies were also initiated to support IND submission and Phase I study.

HPV DNA Vaccine

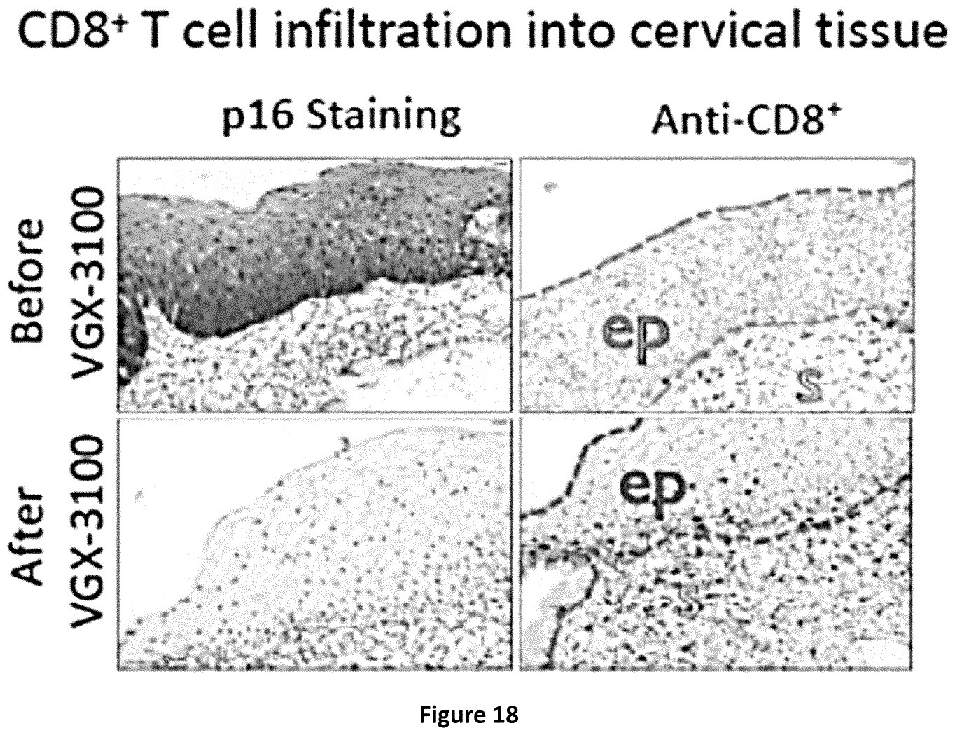

An immunotherapeutic DNA vaccine (VGX-3100) targeting HPV antigens 16/18 has been developed for therapeutic treatment of cervical intraepithelial neoplasia (CIN) (NCT03185013, NCT03721978). In a placebo-controlled phase 2b trial, it was observed that CD8+ T cell infiltration post-DNA vaccination to the site of HPV virus infection. These cellular responses were associated with improved clearance and disease regression (Trimble et al., 2015, Lancet, 386: 2078-2088; FIG. 18). This study demonstrated the anti-viral potential of robust CD8+ T cell responses, generated by DNA vaccination, and provided supportive data for the herein-described post-exposure immunotherapy.

MERS DNA Vaccine

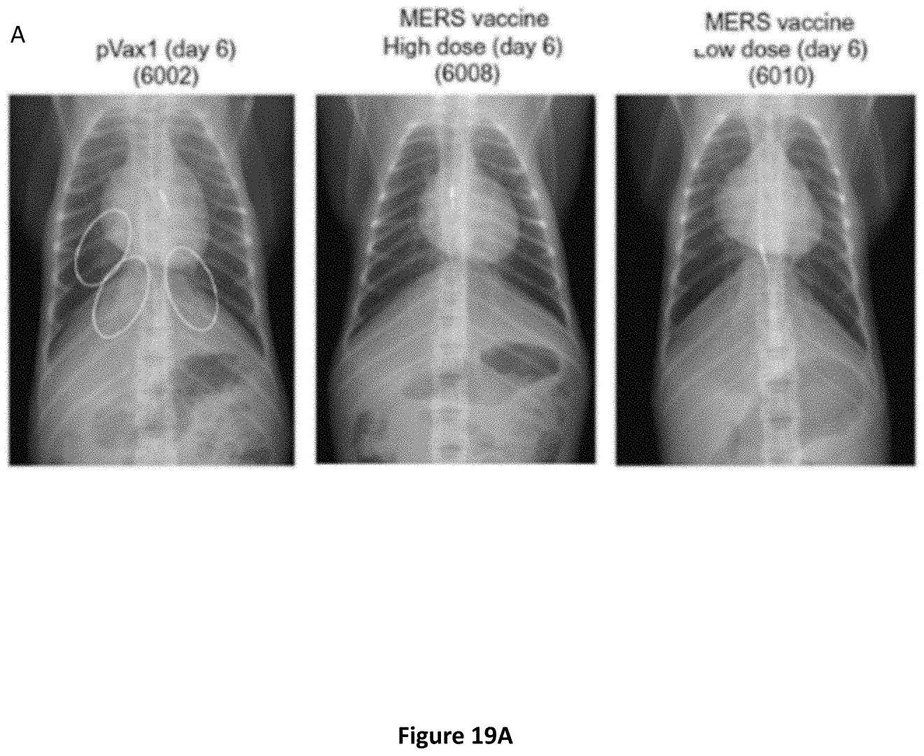

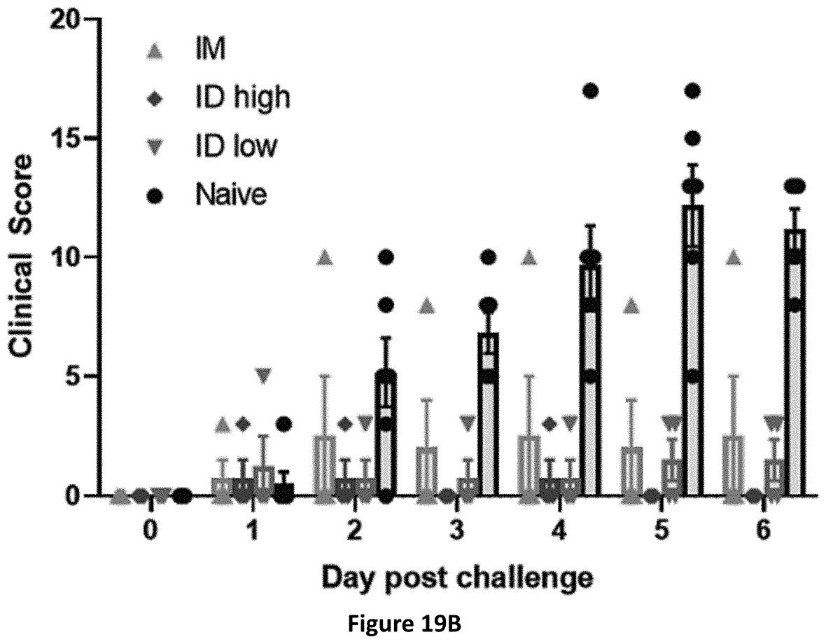

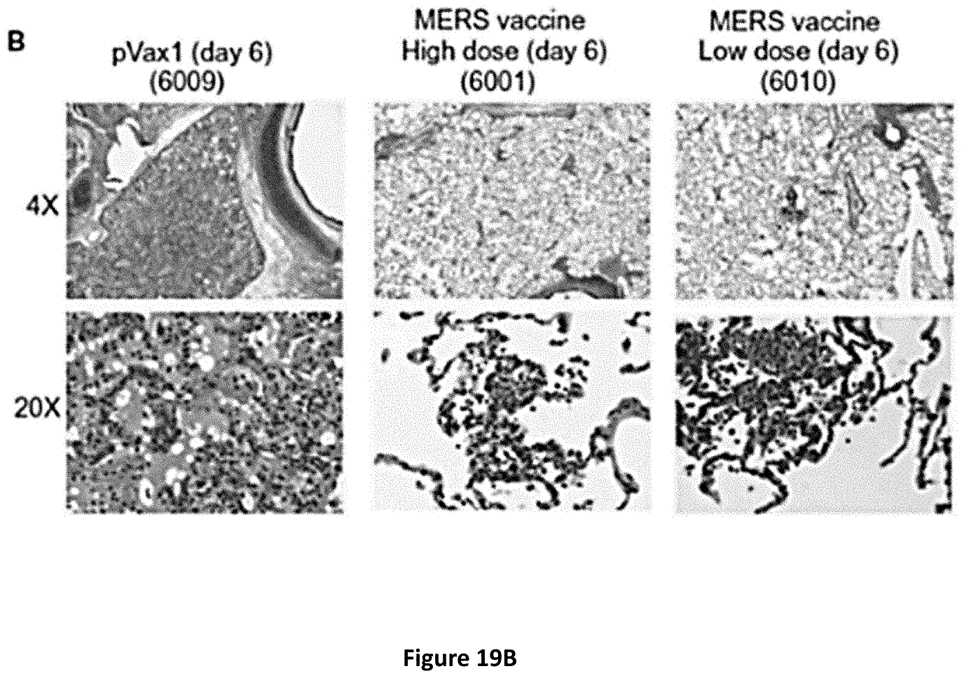

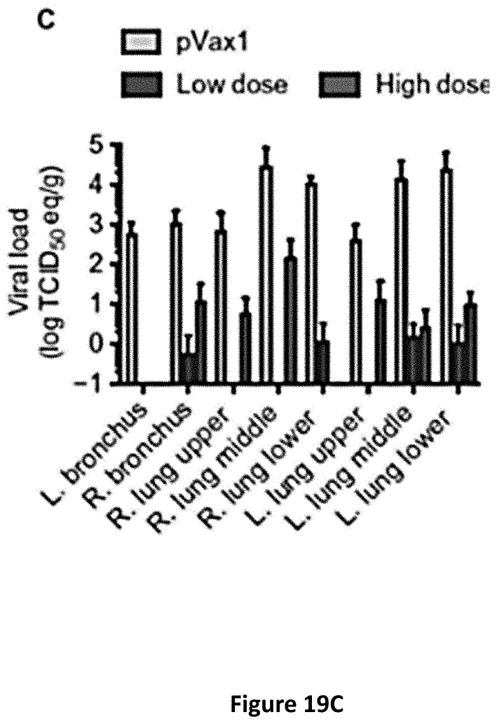

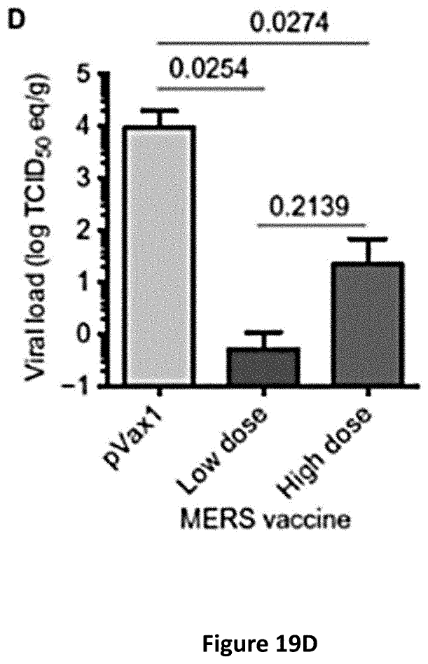

The SARS-CoV-2 vaccine design utilized the experience developing a synthetic DNA vaccine against Middle East Respiratory Syndrome coronavirus (MERS-CoV), a structurally similar betacoronavirus (FIG. 1B). The MERS DNA vaccine was previously demonstrated to induce strong humoral responses, including neutralizing antibodies, and T cell responses in animal models (Muthumani et al., 2015, Sci Transl Med, 7:301ra132). Importantly, the MERS DNA vaccine protected rhesus macaques against a rigorous installation challenge with the MERS-CoV EMC/2012 virus. The protective efficacy of the MERS DNA vaccine was also demonstrated following both intramuscular (IM) delivery and low-dose intradermal (ID) delivery (FIG. 19).

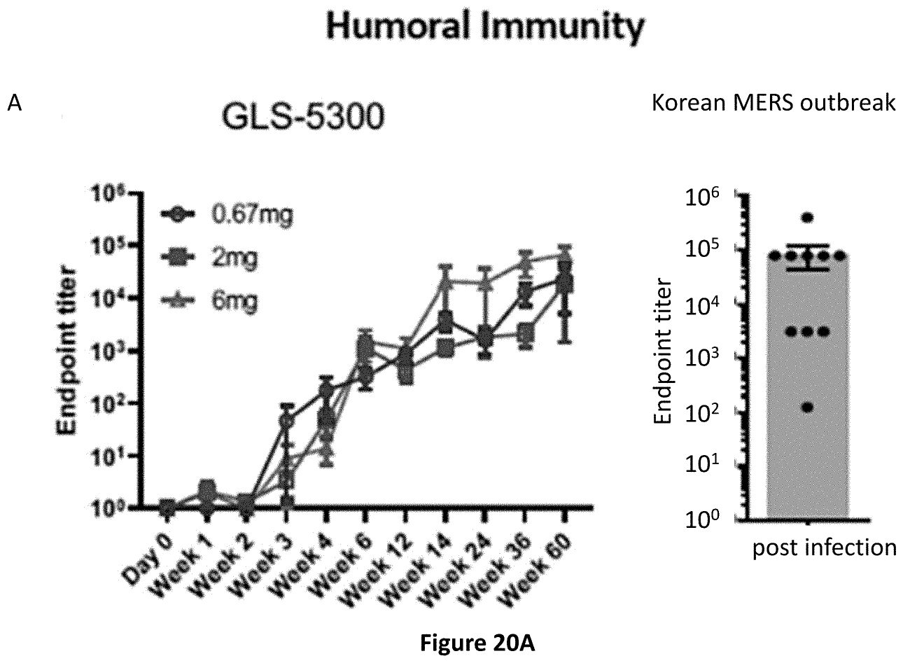

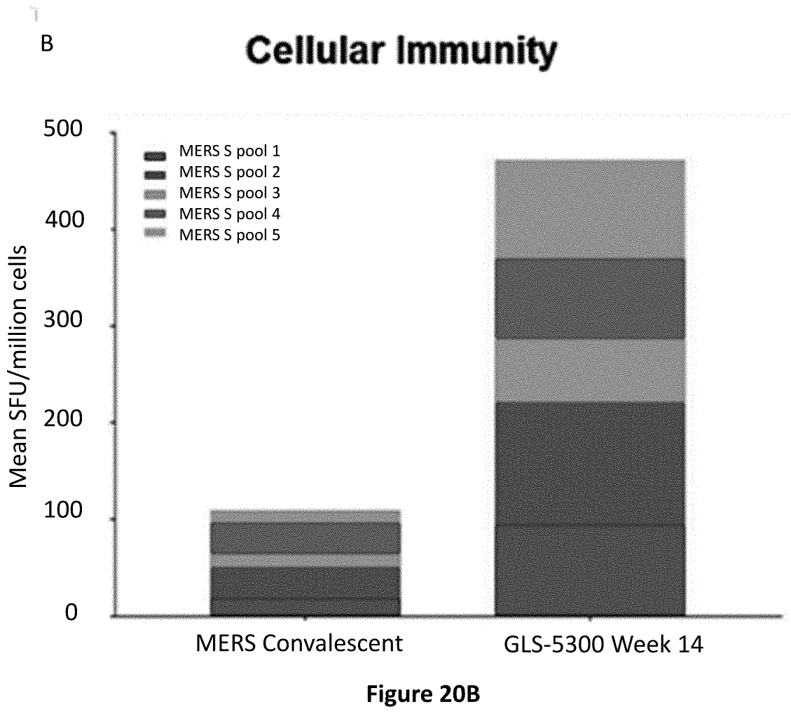

The safety and immunogenicity of the MERS DNA vaccine (GLS-5300) was evaluated in a Phase I study in healthy volunteers (Modjarrad et al, 2019, Lancet Infectious Disease, 19: P1013-1022). It was shown that the levels of neutralizing antibodies in MERS DNA vaccinated participants was not significantly different to those observed in convalescent patients, supporting the passive immunotherapy approach from DNA vaccinated individuals (FIG. 20A). Cellular immune responses were reported to be important for protection against severe MERS disease (Zhao et al., 2017, Sci Immunol, 2:eaan5393). The MERS DNA vaccine induced robust T cell responses compared to MERS convalescent patients (FIG. 20B).

SARS-CoV-2

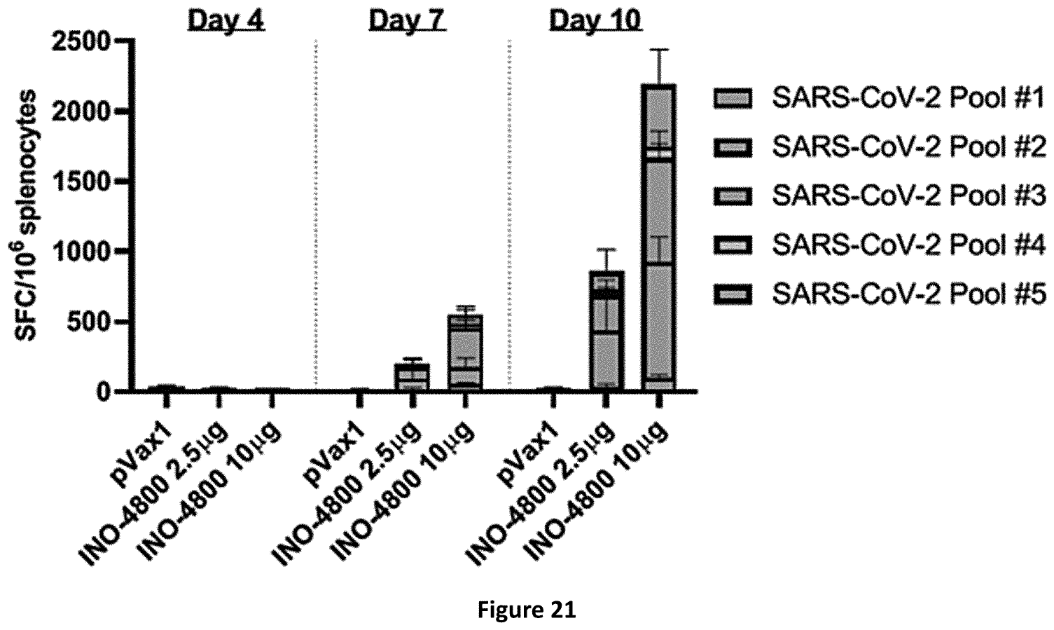

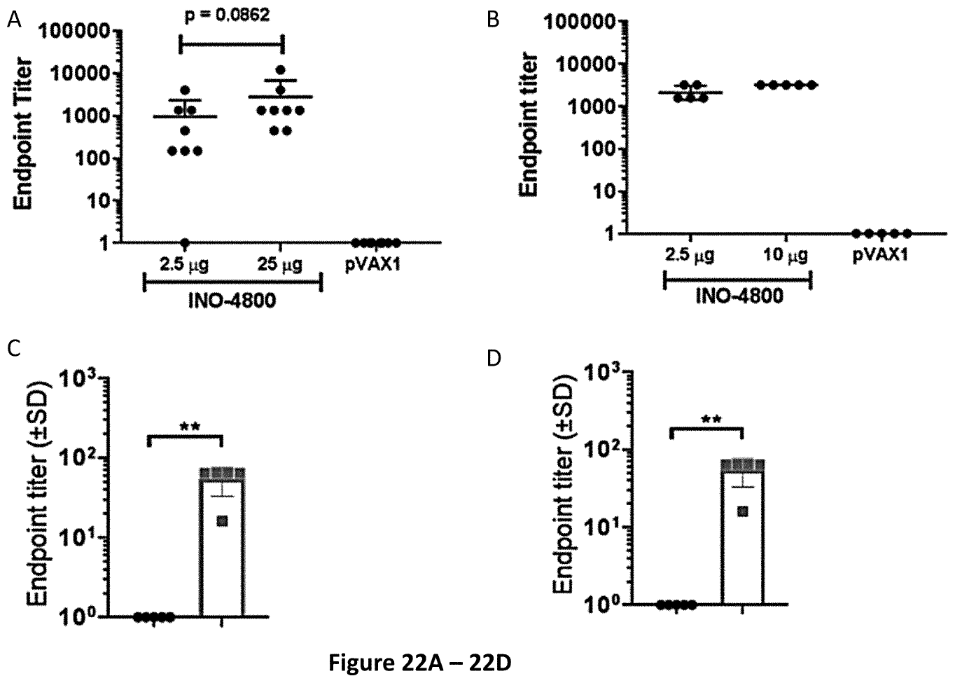

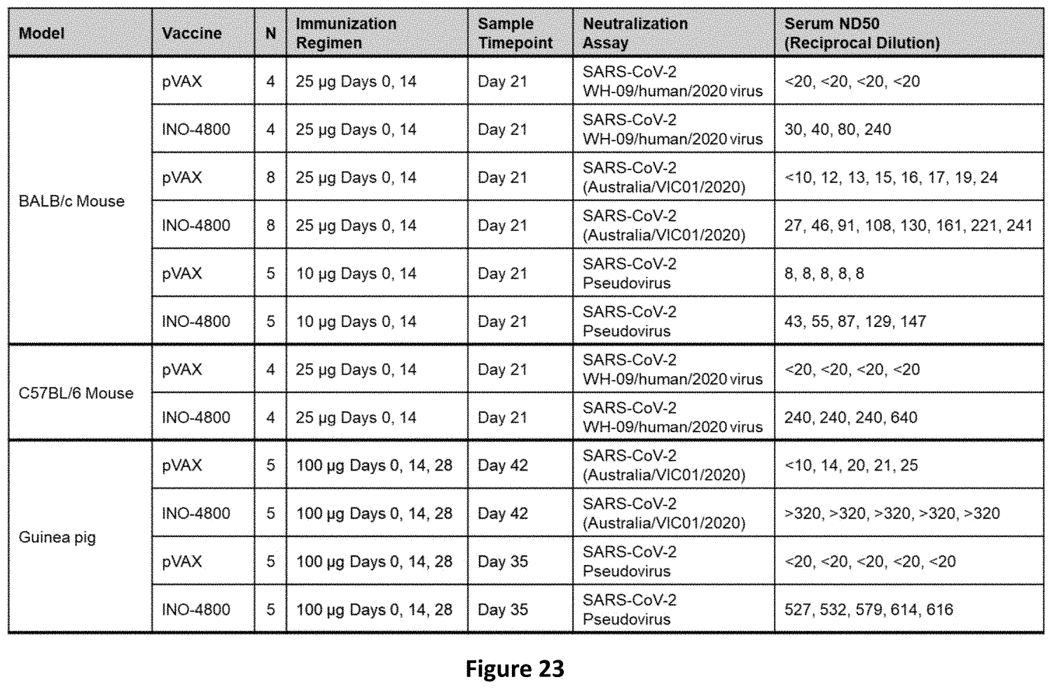

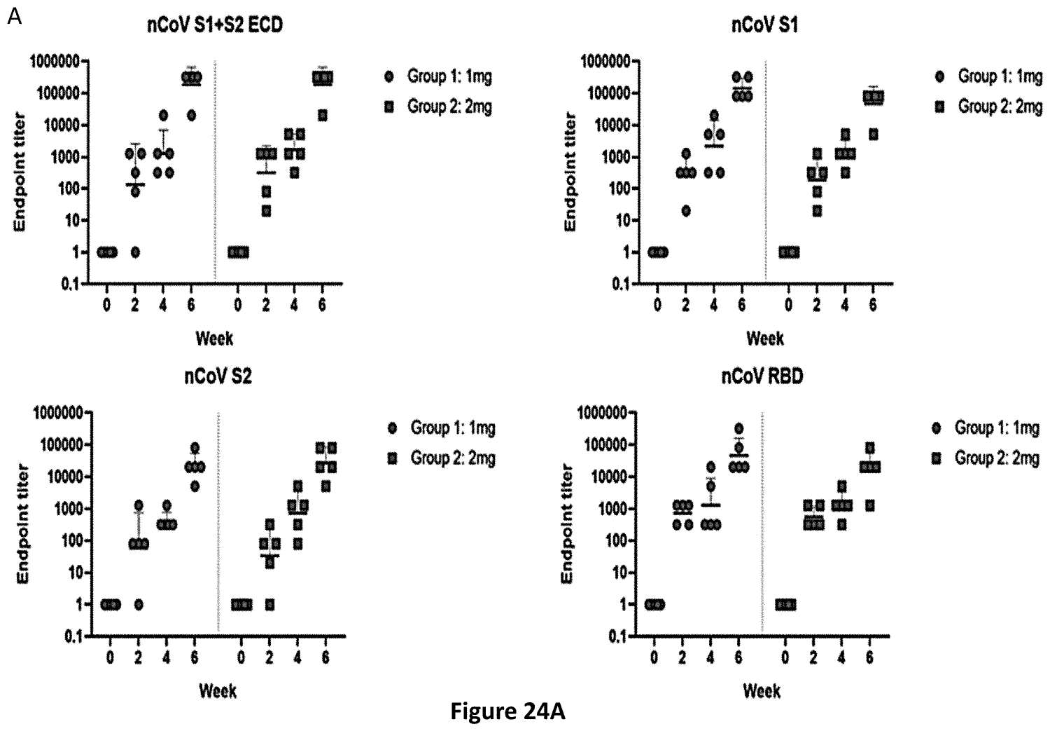

The SARS-CoV-2 DNA vaccine encoded a modified full-length Spike antigen that was optimized for mammalian expression. This vaccine candidate was named INO-4800. Mouse immunogenicity studies were initiated and accelerated studies were performed in guinea pigs, rabbits, and rhesus macaques. The INO-4800 vaccine rapidly induced T cell responses in mice (FIG. 21). INO-4800 induced robust humoral responses against the full-length Spike and RBD in both mice (FIG. 22A and FIG. 22B) and guinea pigs (FIG. 22C and FIG. 22D). Serum neutralizing antibodies (FIG. 23) and blocking of ACE2 were detected.

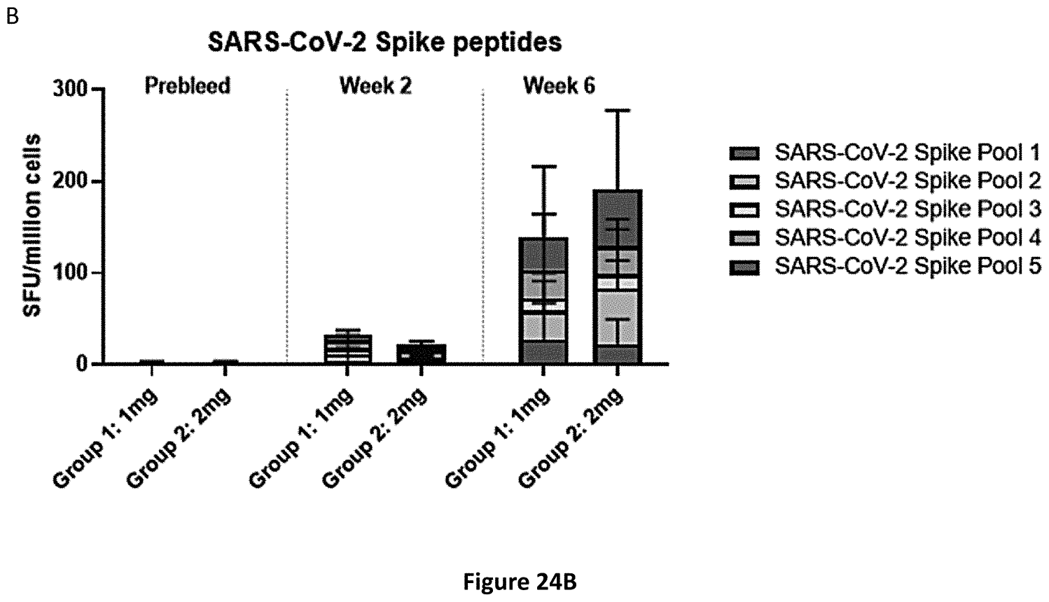

Next, immunogenicity in rhesus macaques was demonstrated, detecting robust antibody titers against the full-length Spike, 51, S2, and RBD (FIG. 24A) and cellular immune responses against SARS-CoV-2 Spike peptides (FIG. 24B). Cross-reactive antibody and T cell responses against SARS-CoV were also observed. Animal challenge models are developed or obtained and are used to assess protective efficacy of the COVID-19 DNA vaccine candidate once these models are available.

### Test/Evaluation

The INO-4800 COVID-19 DNA vaccine was manufactured with thousands of doses available. It is vialed (10 mg/mL vial dose) and 2 mg dosing per treatment time point are suggested.

The present example also includes an additional study in which healthy volunteers are administered an INO-4800 vaccination regimen with INO-4800, which demonstrates the safety of INO-4800 in a larger population. The plasmapheresis samples from these volunteers are also used to purify and manufacture the COVID-19 specific IgG to be utilized in the Phase IIb study.

### Regulatory Strategy

The herein-described technology is highly amenable to accelerated developmental timelines due to the ability to rapidly design the candidate DNA vaccine, manufacture large quantities of the drug product, and leverage previously established regulatory pathways into Phase I clinical evaluation. Furthermore, VGX-3100, a similarly designed DNA medicine to treat HPV 16- and/or HPV-18-related high-grade cervical dysplasia has shown efficacy in humans in a Phase IIb trial.

To facilitate the rapid advancement of INO-4800 into first in human clinical evaluation, the following approach was used:

(1) Utilization of a standard plasmid construction and DNA platform manufacturing process as employed in all previously reviewed IND applications. Plasmid construction and manufacturing process, release, and stability information for 20 Inovio bulk plasmids and 12 plasmid drug products are included in CBER Type II Master File 18468: Platform Manufacturing of DNA Plasmid Products at VGXI Inc.;

(2) Utilization of supportive GLP nonclinical and clinical safety data from similarly designed plasmids detailed in other INDs are cross referenced in the INO-4800 IND application to support the clinical evaluation of INO-4800 as no additional product-specific GLP toxicology or biodistribution studies are planned in line with previous development programs. Clinical data from the Middle East Respiratory Syndrome (MERS), Zika, Lassa Fever, and Ebola vaccine programs also serve as a supportive basis for first in human evaluation of INO-4800;

(3) Utilization of the CELLECTRA® 2000 device for EP which has been used in combination with investigational plasmid DNA products since 2008 in over thirty-four (34) Phase I and Phase II studies worldwide. Over 6900 EP treatments have been delivered using the CELLECTRA® 2000 device following injection of drug product in 1940 subjects. Information on the CELLECTRA® 2000 device is on file with FDA/CBER in Master File 17158;

(4) Conduct of nonclinical immunogenicity studies in mice, guinea pigs, and rabbits. The highest proposed human dose and dosing regimen are evaluated in the rabbit model; and,

(5) Conduct of a first in human Phase I clinical safety and immunogenicity study with features to carefully assess and monitor safety in a first in human setting.

In summary, this platform technology, the same electroporation delivery system, and supportive nonclinical and clinical safety profile has been successfully leveraged to rapidly advance a variety of infectious disease vaccine and immunotherapy candidates into Phase I and II clinical testing.

### Example 3

### Therapies and Prophylaxes to Treat COVID-19

The present example relates, in part, to delivery of a post-exposure combination immune therapy to prevent clinical progression of COVID-19 in patients with a positive diagnosis. COVID-19 and/or SARS-CoV-2 infection has a high risk of significant spread as well as morbidity and mortality. Once patients are diagnosed as PCR (test) positive they must recover-in-place until they are clear of symptoms and are deemed to be COVID-19 negative. Over 80% of such patients recover-in-place over a period of 7-14 days. However, close to 20% eventually progress over a two-week period and require ICU care. A vaccine countermeasure for COVID-19 (INO-4800) has been rapidly developed and moved to the clinic in just 10 weeks. Preclinically, this vaccine induced strong functional antibody responses as well as potent T cell responses. Relationship of the induction of immune responses to viral control is followed. The generation of rapid cellular and antibody responses are likely to improve recovery and reduce progression to severe disease. The herein-described immune therapy, pAI-4800, is comprised of two components (1) passive delivery of immunoglobulin (Ig) from INO-4800 vaccinated participants to COVID-19 positive patients and (2) post-exposure, therapeutic, DNA vaccination with INO-4800.

The scope of the herein-described effort is to develop a platform therapy that can be delivered post-COVID-19 exposure, that prevents the development of advanced disease COVID-19 in the infected individuals.

This study directly addresses the therapies and prophylaxes to treat COVID-19. The objective is to develop a post-exposure immune platform that can be rapidly deployed to prevent SARS-CoV-2 progression to severe COVID-19. The herein-described platform is a combined passive immune sera and post-exposure DNA vaccine approach that is delivered up on COVID-19 diagnosis. More specifically, the contractor develops an immunotherapy to evaluate technical feasibility of immune sera from INO-4800 DNA vaccinated individuals and post-exposure delivery of INO-4800 as therapy in COVID-19 diagnosed patients. This immunotherapy prevents progression to severe COVID-19 disease, with the additional benefit of lowering viral load and also reducing likelihood of virus transmission. Upon a determination that the immunotherapy platform for this transaction has been successfully completed, this platform results in a follow-on production.

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. An immunogenic composition comprising intravenous immunoglobulin (IVIG) generated from one or more host subject vaccinated against Coronavirus disease 2019 (COVID-19) or diagnosed as having COVID-19 and subsequently cured of COVID-19.

2. The composition of claim 1, wherein the host subject was vaccinated with a nucleic acid molecule comprising a nucleotide sequence selected from the group consisting of:
(a) a nucleotide sequence encoding a peptide comprising an amino acid sequence having at least about 90% identity over an entire length of the amino acid sequence set forth in SEQ ID NO:2;
(b) a nucleotide sequence encoding a peptide comprising an amino acid sequence having at least about 90% identity over an entire length of the amino acid sequence set forth in SEQ ID NO:4;
(c) a nucleotide sequence encoding a peptide comprising an amino acid sequence having at least about 90% identity over an entire length of the amino acid sequence set forth in SEQ ID NO:6; and
(d) a nucleotide sequence encoding a peptide comprising an amino acid sequence having at least about 90% identity over an entire length of the amino acid sequence set forth in SEQ ID NO:8.

3. The immunogenic composition of claim 1, wherein the host subject was vaccinated with a nucleic acid molecule comprising a nucleotide sequence encoding the amino acid sequence selected from the group consisting of SEQ ID NO:2, SEQ ID NO:4, SEQ ID NO:6 and SEQ ID NO:8.

4. The immunogenic composition of claim 1, wherein the host subject was vaccinated with a nucleic acid molecule comprising a nucleotide sequence selected from the group consisting of:
(a) the nucleotide sequence having at least about 90% identity over an entire length of the nucleic acid sequence set forth in SEQ ID NO:1;
(b) the nucleotide sequence having at least about 90% identity over an entire length of the nucleic acid sequence set forth in SEQ ID NO:3;
(c) the nucleotide sequence having at least about 90% identity over an entire length of the nucleic acid sequence set forth in SEQ ID NO:5; and
(d) the nucleotide sequence having at least about 90% identity over an entire length of the nucleic acid sequence set forth in SEQ ID NO:7.

5. The immunogenic composition of claim 1, wherein the nucleic acid molecule comprises the nucleic acid sequence selected from the group consisting of SEQ ID NO:1, SEQ ID NO:3, SEQ ID NO:5 and SEQ ID NO:7.

6. The immunogenic composition of claim 1, wherein the immunoglobulin is extracted from a biological sample.

7. The immunogenic composition of claim 6, wherein the biological sample is a plasma, blood, or a combination thereof.

8. The immunogenic composition of claim 1, further comprising a nucleic acid molecule comprising a nucleotide sequence selected from the group consisting of:
(a) a nucleotide sequence encoding a peptide comprising an amino acid sequence having at least about 90% identity over an entire length of the amino acid sequence set forth in SEQ ID NO:2;
(b) a nucleotide sequence encoding a peptide comprising an amino acid sequence having at least about 90% identity over an entire length of the amino acid sequence set forth in SEQ ID NO:4;
(c) a nucleotide sequence encoding a peptide comprising an amino acid sequence having at least about 90% identity over an entire length of the amino acid sequence set forth in SEQ ID NO:6; and
(d) a nucleotide sequence encoding a peptide comprising an amino acid sequence having at least about 90% identity over an entire length of the amino acid sequence set forth in SEQ ID NO:8.

9. The immunogenic composition of claim 8, wherein the nucleic acid molecule comprises a nucleotide sequence encoding the amino acid sequence selected from the group consisting of SEQ ID NO:2, SEQ ID NO:4, SEQ ID NO:6 and SEQ ID NO:8.

10. The immunogenic composition of claim 8, wherein the nucleic acid molecule comprises a nucleotide sequence selected from the group consisting of:
(a) the nucleotide sequence having at least about 90% identity over an entire length of the nucleic acid sequence set forth in SEQ ID NO:1;
(b) the nucleotide sequence having at least about 90% identity over an entire length of the nucleic acid sequence set forth in SEQ ID NO:3;
(c) the nucleotide sequence having at least about 90% identity over an entire length of the nucleic acid sequence set forth in SEQ ID NO:5; and
(d) the nucleotide sequence having at least about 90% identity over an entire length of the nucleic acid sequence set forth in SEQ ID NO:7.

11. The immunogenic composition of claim 8, wherein the nucleic acid molecule comprises the nucleic acid sequence selected from the group consisting of SEQ ID NO:1, SEQ ID NO:3, SEQ ID NO:5 and SEQ ID NO:7.

12. The immunogenic composition of claim 1, wherein the nucleic acid molecule comprises an expression vector.

13. The immunogenic composition of claim 1, wherein the nucleic acid molecule is incorporated into a viral particle.

14. The immunogenic composition of claim 1, further comprising a pharmaceutically acceptable excipient.

15. The immunogenic composition of claim 1, further comprising an adjuvant.

16. A method of generating an immunogenic composition comprising intravenous immunoglobulin (IVIG) from one or more host subject, the method comprising:
a) administering a nucleic acid molecule encoding a SARS-CoV-2 antigen to the host subject; and
b) isolating a biological sample comprising one or more immunoglobulin molecule from the host subject.

17. The method of claim 16, wherein the subject has been diagnosed as having SARS-CoV-2 infection or COVID-19, or has is considered cured of SARS-CoV-2 infection or COVID-19.

18. The method of claim 16, wherein the nucleic acid molecule comprises a nucleotide sequence selected from the group consisting of:
(a) a nucleotide sequence encoding a peptide comprising an amino acid sequence having at least about 90% identity over an entire length of the amino acid sequence set forth in SEQ ID NO:2;
(b) a nucleotide sequence encoding a peptide comprising an amino acid sequence having at least about 90% identity over an entire length of the amino acid sequence set forth in SEQ ID NO:4;
(c) a nucleotide sequence encoding a peptide comprising an amino acid sequence having at least about 90% identity over an entire length of the amino acid sequence set forth in SEQ ID NO:6; and
(d) a nucleotide sequence encoding a peptide comprising an amino acid sequence having at least about 90% identity over an entire length of the amino acid sequence set forth in SEQ ID NO:8.

19. The method of claim 16, wherein the nucleic acid molecule comprises a nucleotide sequence encoding the amino acid sequence selected from the group consisting of SEQ ID NO:2, SEQ ID NO:4, SEQ ID NO:6 and SEQ ID NO:8.

20. The method of claim 16, wherein the nucleic acid molecule comprises a nucleotide sequence selected from the group consisting of:
(a) the nucleotide sequence having at least about 90% identity over an entire length of the nucleic acid sequence set forth in SEQ ID NO:1;
(b) the nucleotide sequence having at least about 90% identity over an entire length of the nucleic acid sequence set forth in SEQ ID NO:3;
(c) the nucleotide sequence having at least about 90% identity over an entire length of the nucleic acid sequence set forth in SEQ ID NO:5; and
(d) the nucleotide sequence having at least about 90% identity over an entire length of the nucleic acid sequence set forth in SEQ ID NO:7.

21. The method of claim 16, wherein the nucleic acid molecule comprises the nucleic acid sequence selected from the group consisting of SEQ ID NO:1, SEQ ID NO:3, SEQ ID NO:5 and SEQ ID NO:7.

22. The method of claim 16, wherein the biological sample is a plasma, blood, or a combination thereof.

23. A method of inducing an immune response against SARS-CoV-2 in a subject in need thereof, the method comprising administering an immunogenic composition of claim 1 to the subject.

24. The method of claim 23, further comprising administering a nucleic acid molecule encoding a SARS-CoV-2 antigen to the subject.

25. The method of claim 24, wherein the nucleic acid molecule comprises a nucleotide sequence selected from the group consisting of:
(a) a nucleotide sequence encoding a peptide comprising an amino acid sequence having at least about 90% identity over an entire length of the amino acid sequence set forth in SEQ ID NO:2;
(b) a nucleotide sequence encoding a peptide comprising an amino acid sequence having at least about 90% identity over an entire length of the amino acid sequence set forth in SEQ ID NO:4;
(c) a nucleotide sequence encoding a peptide comprising an amino acid sequence having at least about 90% identity over an entire length of the amino acid sequence set forth in SEQ ID NO:6; and
(d) a nucleotide sequence encoding a peptide comprising an amino acid sequence having at least about 90% identity over an entire length of the amino acid sequence set forth in SEQ ID NO:8.

26. The method of claim 24, wherein the nucleic acid molecule comprises a nucleotide sequence encoding the amino acid sequence selected from the group consisting of SEQ ID NO:2, SEQ ID NO:4, SEQ ID NO:6 and SEQ ID NO:8.

27. The method of claim 24, wherein the nucleic acid molecule comprises a nucleotide sequence selected from the group consisting of:
(a) the nucleotide sequence having at least about 90% identity over an entire length of the nucleic acid sequence set forth in SEQ ID NO:1;
(b) the nucleotide sequence having at least about 90% identity over an entire length of the nucleic acid sequence set forth in SEQ ID NO:3;
(c) the nucleotide sequence having at least about 90% identity over an entire length of the nucleic acid sequence set forth in SEQ ID NO:5; and
(d) the nucleotide sequence having at least about 90% identity over an entire length of the nucleic acid sequence set forth in SEQ ID NO:7.

28. The method of claim 24, wherein the nucleic acid molecule comprises the nucleic acid sequence selected from the group consisting of SEQ ID NO:1, SEQ ID NO:3, SEQ ID NO:5 and SEQ ID NO:7.

29. The method of claim 24, wherein administering includes at least one of electroporation and injection.

30. A method of protecting a subject in need thereof from infection with SARS-CoV-2 or COVID-19, the method comprising administering an immunogenic composition of claim 1 to the subject.

31. The method of claim 30, further comprising administering a nucleic acid molecule encoding a SARS-CoV-2 antigen to the subject.

32. The method of claim 30, wherein the nucleic acid molecule comprises a nucleotide sequence selected from the group consisting of:
(a) a nucleotide sequence encoding a peptide comprising an amino acid sequence having at least about 90% identity over an entire length of the amino acid sequence set forth in SEQ ID NO:2;
(b) a nucleotide sequence encoding a peptide comprising an amino acid sequence having at least about 90% identity over an entire length of the amino acid sequence set forth in SEQ ID NO:4;
(c) a nucleotide sequence encoding a peptide comprising an amino acid sequence having at least about 90% identity over an entire length of the amino acid sequence set forth in SEQ ID NO:6; and
(d) a nucleotide sequence encoding a peptide comprising an amino acid sequence having at least about 90% identity over an entire length of the amino acid sequence set forth in SEQ ID NO:8.

33. The method of claim 30, wherein the nucleic acid molecule comprises a nucleotide sequence encoding the amino acid sequence selected from the group consisting of SEQ ID NO:2, SEQ ID NO:4, SEQ ID NO:6 and SEQ ID NO:8.

34. The method of claim 30, wherein the nucleic acid molecule comprises a nucleotide sequence selected from the group consisting of:
(a) the nucleotide sequence having at least about 90% identity over an entire length of the nucleic acid sequence set forth in SEQ ID NO:1;
(b) the nucleotide sequence having at least about 90% identity over an entire length of the nucleic acid sequence set forth in SEQ ID NO:3;
(c) the nucleotide sequence having at least about 90% identity over an entire length of the nucleic acid sequence set forth in SEQ ID NO:5; and
(d) the nucleotide sequence having at least about 90% identity over an entire length of the nucleic acid sequence set forth in SEQ ID NO:7.

35. The method of claim 30, wherein the nucleic acid molecule comprises the nucleic acid sequence selected from the group consisting of SEQ ID NO:1, SEQ ID NO:3, SEQ ID NO:5 and SEQ ID NO:7.

36. The method of claim 30, wherein administering includes at least one of electroporation and injection.


SEQUENCE LISTING (20 sequences)

SEQ ID NO: 1 - DNA, 3783 bp, Artificial Sequnce
  Chemically Synthesized, Spike
agccagtgtgtgaacctgaccaccagaacacagctgcctcctgcctacaccaacagcttcaccagaggagtctactacccagacaaagtcttcagaagctctgtgctgcacagcacccaggacctgttcctgcctttcttcagcaacgtgacctggttccacgccatccacgtgtctggcaccaacggcaccaagagatttgacaaccctgttcttcctttcaatgatggcgtgtactttgccagcacagagaagagcaacatcatccgaggctggatctttggcaccaccctggacagcaaaacccagagcctgctgatcgtgaacaacgccaccaacgtggtcatcaaggtgtgtgagttccagttctgcaatgaccctttcctgggcgtgtactaccacaagaacaacaagtcctggatggagtctgagttcagagtctacagctctgccaacaactgcacatttgaatatgtgtcccagcctttcctgatggacctggagggcaagcagggcaactttaagaacctgagagaatttgtgttcaagaacatcgatggctacttcaagatctacagcaagcacacacccatcaacctggtgagagacctgcctcagggcttctctgccctggagcctctggtggacctgcccatcggcatcaacatcaccagattccagaccctgctggccctgcacagaagctacctgaccccaggagacagcagcagcggctggacagctggagctgctgcctactacgtgggctacctgcagcccaggaccttcctgctgaagtacaacgaaaatggcaccatcacagatgctgttgactgtgccctggaccctcttagcgagaccaagtgcaccctgaagtccttcacagtggagaaaggcatctaccagaccagcaacttccgagtgcagccaacagagagcatcgtgagatttccaaacatcaccaacctgtgcccttttggagaagtcttcaatgccaccagatttgcttctgtgtacgcctggaacagaaaaagaatcagcaactgtgtggctgactactctgtgctgtacaactctgcctccttctccaccttcaagtgctatggagtctctccaaccaagctgaatgacctgtgcttcaccaacgtgtatgctgacagctttgtgatcagaggagatgaagtgcggcagattgctcctggccagacaggcaagattgctgactacaactacaagctgcctgatgacttcacaggctgtgtcatcgcctggaacagcaacaacctggacagcaaggtgggcggcaactacaactacctgtacagacttttcaggaagagcaacctgaagccttttgaaagagacatctccacagagatctaccaggctggcagcacaccctgcaatggtgtggaaggcttcaactgctacttccctctgcagagctacggcttccagccaacaaatggcgtgggctaccagccttacagagtggtggtgctgtcctttgagctgctgcacgcccctgccacagtgtgtggccccaagaagagcaccaacctggtgaagaacaaatgtgtgaacttcaatttcaatggcctgacaggcacaggagtgctgacagagagcaacaagaagtttcttcctttccagcagtttggaagagacattgctgacaccacagatgctgtgagagatcctcagaccctggagatcctggatatcacaccctgctcctttggaggagtttctgtcatcacacctggcaccaataccagcaaccaagtggctgtgctgtaccaagatgtgaattgcacagaagtgcctgtggccatccacgctgaccagctgacacccacctggagagtgtacagcacaggcagcaatgttttccagacaagagctggctgcctgattggagcagagcacgtgaacaacagctatgaatgtgacatccctattggagctggcatctgtgccagctaccagacccaaaccaacagcccaagaagagccagatctgtggccagccagagcatcatcgcctacaccatgagcctgggagctgagaactctgtggcctacagcaacaacagcatcgccatccccaccaacttcaccatctctgtgaccacagagatcctgcctgtgtccatgaccaagacatctgtggactgcaccatgtacatctgtggagacagcacagaatgcagcaacctgctgctgcagtacggctccttctgcacccagctgaacagagccctgacaggcatcgctgtggagcaggacaagaacacacaggaagtgtttgcccaggtgaagcagatctacaaaacaccacccatcaaggactttggaggcttcaatttctcccaaatcctgcctgaccccagcaagccttccaagagaagcttcattgaagacctgctgttcaacaaagtgaccctggctgatgctggcttcatcaagcagtatggagactgcctgggagacattgctgccagagacctgatctgtgcccagaagtttaatggcctgactgtgctgcctcctctgctgacagatgaaatgatcgcccagtacacatctgccctgctggctggcaccatcaccagtggctggacatttggagctggagctgccctgcagatcccttttgccatgcagatggcctacagatttaatggcatcggcgtgacccagaacgtgctgtacgagaaccagaagctgatcgccaaccagttcaactctgccatcggcaagatccaggacagcctgagcagcacagcctctgccctgggcaagctgcaggatgtggtgaaccaaaacgcccaggccctgaacaccctggtgaagcagctgagcagcaactttggagccatctcctctgtgctgaatgacatcctgagccggctggacaaggtggaagcagaagtgcagatcgacagactcatcacaggccgcctgcagagcctgcagacctacgtgacccagcagctgatcagagctgctgagatccgggcctctgccaacctggctgccaccaagatgtcagaatgtgtgctgggccagagcaaaagagtggacttctgtggcaaaggctaccacctgatgtccttccctcagtctgctcctcacggcgtggtgttcctgcacgtgacctacgtgcctgcccaggagaagaacttcaccacagctcctgccatctgccacgatggcaaggcccacttcccaagagaaggtgtctttgtgtccaatggcacccactggttcgtgacccagagaaacttctacgagcctcagatcatcaccacagacaacacatttgtgtctggcaactgtgatgtggtcatcggcatcgtgaacaacacagtttatgaccctctgcagcctgagctggacagcttcaaagaagagctggacaagtacttcaagaaccacacatctccagatgtggacctgggagacatctctggcatcaatgcctctgtggtgaacatccagaaggaaattgacaggctgaacgaagtggccaagaacctgaacgaaagcctcatcgacctgcaggagctgggcaagtacgagcagtacatcaagtggccttggtacatctggctgggcttcatcgctggcctcatcgccatcgtgatggtgaccatcatgctgtgctgcatgaccagctgctgctcttgcctgaagggctgctgcagctgtggcagctgctgcaagtttgatgaagatgactctgagcctgtgctgaagggcgtgaagctgcactacaca

SEQ ID NO: 2 - protein, 1261 aa, Artificial Sequnce
  Chemically Synthesized, Spike
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - DNA, 3843 bp, Artificial Sequnce
  Chemically Synthesized, Spike with IgE leader
atggattggacttggattctctttctcgttgctgcagccacacgcgttcatagcagccagtgtgtgaacctgaccaccagaacacagctgcctcctgcctacaccaacagcttcaccagaggagtctactacccagacaaagtcttcagaagctctgtgctgcacagcacccaggacctgttcctgcctttcttcagcaacgtgacctggttccacgccatccacgtgtctggcaccaacggcaccaagagatttgacaaccctgttcttcctttcaatgatggcgtgtactttgccagcacagagaagagcaacatcatccgaggctggatctttggcaccaccctggacagcaaaacccagagcctgctgatcgtgaacaacgccaccaacgtggtcatcaaggtgtgtgagttccagttctgcaatgaccctttcctgggcgtgtactaccacaagaacaacaagtcctggatggagtctgagttcagagtctacagctctgccaacaactgcacatttgaatatgtgtcccagcctttcctgatggacctggagggcaagcagggcaactttaagaacctgagagaatttgtgttcaagaacatcgatggctacttcaagatctacagcaagcacacacccatcaacctggtgagagacctgcctcagggcttctctgccctggagcctctggtggacctgcccatcggcatcaacatcaccagattccagaccctgctggccctgcacagaagctacctgaccccaggagacagcagcagcggctggacagctggagctgctgcctactacgtgggctacctgcagcccaggaccttcctgctgaagtacaacgaaaatggcaccatcacagatgctgttgactgtgccctggaccctcttagcgagaccaagtgcaccctgaagtccttcacagtggagaaaggcatctaccagaccagcaacttccgagtgcagccaacagagagcatcgtgagatttccaaacatcaccaacctgtgcccttttggagaagtcttcaatgccaccagatttgcttctgtgtacgcctggaacagaaaaagaatcagcaactgtgtggctgactactctgtgctgtacaactctgcctccttctccaccttcaagtgctatggagtctctccaaccaagctgaatgacctgtgcttcaccaacgtgtatgctgacagctttgtgatcagaggagatgaagtgcggcagattgctcctggccagacaggcaagattgctgactacaactacaagctgcctgatgacttcacaggctgtgtcatcgcctggaacagcaacaacctggacagcaaggtgggcggcaactacaactacctgtacagacttttcaggaagagcaacctgaagccttttgaaagagacatctccacagagatctaccaggctggcagcacaccctgcaatggtgtggaaggcttcaactgctacttccctctgcagagctacggcttccagccaacaaatggcgtgggctaccagccttacagagtggtggtgctgtcctttgagctgctgcacgcccctgccacagtgtgtggccccaagaagagcaccaacctggtgaagaacaaatgtgtgaacttcaatttcaatggcctgacaggcacaggagtgctgacagagagcaacaagaagtttcttcctttccagcagtttggaagagacattgctgacaccacagatgctgtgagagatcctcagaccctggagatcctggatatcacaccctgctcctttggaggagtttctgtcatcacacctggcaccaataccagcaaccaagtggctgtgctgtaccaagatgtgaattgcacagaagtgcctgtggccatccacgctgaccagctgacacccacctggagagtgtacagcacaggcagcaatgttttccagacaagagctggctgcctgattggagcagagcacgtgaacaacagctatgaatgtgacatccctattggagctggcatctgtgccagctaccagacccaaaccaacagcccaagaagagccagatctgtggccagccagagcatcatcgcctacaccatgagcctgggagctgagaactctgtggcctacagcaacaacagcatcgccatccccaccaacttcaccatctctgtgaccacagagatcctgcctgtgtccatgaccaagacatctgtggactgcaccatgtacatctgtggagacagcacagaatgcagcaacctgctgctgcagtacggctccttctgcacccagctgaacagagccctgacaggcatcgctgtggagcaggacaagaacacacaggaagtgtttgcccaggtgaagcagatctacaaaacaccacccatcaaggactttggaggcttcaatttctcccaaatcctgcctgaccccagcaagccttccaagagaagcttcattgaagacctgctgttcaacaaagtgaccctggctgatgctggcttcatcaagcagtatggagactgcctgggagacattgctgccagagacctgatctgtgcccagaagtttaatggcctgactgtgctgcctcctctgctgacagatgaaatgatcgcccagtacacatctgccctgctggctggcaccatcaccagtggctggacatttggagctggagctgccctgcagatcccttttgccatgcagatggcctacagatttaatggcatcggcgtgacccagaacgtgctgtacgagaaccagaagctgatcgccaaccagttcaactctgccatcggcaagatccaggacagcctgagcagcacagcctctgccctgggcaagctgcaggatgtggtgaaccaaaacgcccaggccctgaacaccctggtgaagcagctgagcagcaactttggagccatctcctctgtgctgaatgacatcctgagccggctggacaaggtggaagcagaagtgcagatcgacagactcatcacaggccgcctgcagagcctgcagacctacgtgacccagcagctgatcagagctgctgagatccgggcctctgccaacctggctgccaccaagatgtcagaatgtgtgctgggccagagcaaaagagtggacttctgtggcaaaggctaccacctgatgtccttccctcagtctgctcctcacggcgtggtgttcctgcacgtgacctacgtgcctgcccaggagaagaacttcaccacagctcctgccatctgccacgatggcaaggcccacttcccaagagaaggtgtctttgtgtccaatggcacccactggttcgtgacccagagaaacttctacgagcctcagatcatcaccacagacaacacatttgtgtctggcaactgtgatgtggtcatcggcatcgtgaacaacacagtttatgaccctctgcagcctgagctggacagcttcaaagaagagctggacaagtacttcaagaaccacacatctccagatgtggacctgggagacatctctggcatcaatgcctctgtggtgaacatccagaaggaaattgacaggctgaacgaagtggccaagaacctgaacgaaagcctcatcgacctgcaggagctgggcaagtacgagcagtacatcaagtggccttggtacatctggctgggcttcatcgctggcctcatcgccatcgtgatggtgaccatcatgctgtgctgcatgaccagctgctgctcttgcctgaagggctgctgcagctgtggcagctgctgcaagtttgatgaagatgactctgagcctgtgctgaagggcgtgaagctgcactacacataatga

SEQ ID NO: 4 - protein, 1279 aa, Artificial Sequnce
  Chemically Synthesized, Spike with IgE leader
MDWTWILFLVAAATRVHSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 5 - DNA, 3783 bp, Artificial Sequnce
  Chemically Synthesized, Spike OL
agccagtgtgtgaacctgaccaccagaacacagctgcctcctgcctacaccaacagcttcaccagaggagtctactacccagacaaggtgttcagaagctctgtgctgcacagcacccaggacctcttcctgcctttcttcagcaacgtgacctggttccacgccatccacgtgtctggcaccaacggcaccaagagatttgacaaccctgtgctgcctttcaatgatggtgtgtactttgccagcacagagaagagcaacatcatccgaggctggatctttggcaccaccctggacagcaaaacacagagcctgctgatcgtgaataatgccaccaacgtggtcatcaaggtgtgtgagttccagttctgcaatgaccctttcctgggcgtgtactaccacaagaacaacaagtcctggatggagtctgagttccgagtgtacagctctgccaacaactgcacatttgaatatgtgtcccagcctttcctgatggacctggagggcaagcagggcaatttcaagaacctgagagaatttgtgttcaagaacatcgatggctacttcaagatctacagcaagcacacacccatcaacctggtgagagatcttcctcagggcttctctgccctggagcctctggtggacctgcccatcggcatcaacatcacccgctttcagaccctgctggccctgcacagaagctacctgaccccaggagacagcagcagcggctggacagctggagctgctgcctactacgtgggctacctgcagccaagaaccttcctgctgaagtacaacgaaaatggcaccatcactgtggctgtggcctgtgccctggaccctctttctgagaccaagtgcaccctgaagtccttcacagtggagaaaggcatctaccagaccagcaacttcagagttcagccaacagagagcatcgtgagatttccaaacatcaccaacctgtgtccttttggagaagtcttcaatgccaccagatttgcttctgtgtacgcctggaacagaaaaagaatcagcaactgtgtggctgactactctgtgctgtacaactctgcctccttctccaccttcaagtgctacggtgtgtctcctaccaagctgaatgacctgtgcttcaccaacgtgtatgctgacagctttgtcatcagaggagatgaagtgcggcagatcgcccctggccagacaggcaagattgctgactacaactacaagctgcctgatgacttcacaggctgtgtcatcgcctggaacagcaacaacctggacagcaaggtgggcggcaactacaactacctgtacagacttttcaggaagagcaacctgaagccttttgaaagagacatctccacagagatctaccaggctggcagcacaccctgcaatggagtggaaggcttcaactgctacttccctctgcagagctacggcttccagcccaccaatggcgtgggctaccagccttacagagtggtggtgctgtcctttgagctgctgcacgcccctgccacagtgtgtggccccaagaagagcaccaacctggtgaagaacaaatgtgtgaacttcaatttcaatggcctgacaggcacaggagtgctgacagagagcaacaagaagttcctgcctttccagcagtttggaagagacattgctgacaccacagatgctgtgagagatcctcagaccctggagatcctggacatcacaccctgctcctttggaggagtttctgtcatcacacctggagccaacaccagcaaccaagtgacagtgctgtaccaagatgtgaactgcacagaagttcctgtggccatccacgctgaccagctgaccccaacctggagagtctacagcacaggcagcaacgtgtttaaaacaagagctggctgcctgattggagcagagcacgtgaacaacagctatgaatgtgacatccctattggagctggcatctgtgccagctaccagacccaaaccaacagcccaagaagagccaggagcacagccagccagagcatcatcgcctacaccatgagcctgggagcagagaactctgtggcctacagcaacaacagcatcgtcatccccaccaacttcaccatctctgtgaccacagagatcctgcctgtgtccatgaccaagacatctgtggactgcaccatgtacatctgcagtgacagcacagaatgcagcaaccctctgctgcagtacggctccttctgcacccagctgaacagagccctgacaggcatcgctgtggagcaggacaagaacacacaggaagtgtttgcccaggtgaagcagatctacaaaacaccacccatcaaggactttggaggcttcaacttctcccagatcctgcctgaccccagcaagcccagcaagagaagcttcattgaagacctgctgttcaacaaagtgaccctggctgatgctggcttcatcaaacaatatggagactgcctgggagacattgctgccagagacctgatctgtgcccagaagtttaatggcctgactgtgctgcctcctctgctgacagatgaaatgatcgcccagtacacatctgccctgctggctggcaccatcacatctggctggacatttggagctggagctgccctgcagatcccttttgccatgcagatggcctacagatttaatggcatcagagtgacccagaacgtgctgtatgaaaaccagaagctgatcgccaaccagttcaactctgccatcggcaagatccaggacagcctgagcagcacagcctctgccctgggcaagctgcaggatgtggtgaaccaaaatgcccaggccctgaacaccctggtgaagcagctgagcagcaccttctccaccatctccagcgtgctgaatgacatcctgagccggctggacaaggtggaagctgaggtgcagatcgacagactcatcacaggccggctgcagagcctgcagacctacgtgacccagcagctgatcagagctgctgagatcagagcttctgccaacctgaaggccaccaagatgtcagaatgtgtgctgggccagagcaagagagtggacttctgtggcaaaggctaccacctgatgtccttccctcagtctgctcctcacggcgtggtgttcctgcacgtgacctacgtgcctgcccaggagaagaacttcaccacagctcctgccacctgccacgatggcaaagcccacttcccaagagaaggcgtctttgtgtccaatggcacccactggttcgtgacccagagaaactttgatgagcctcagatcatcaccacagacaacacatttgtttctggcaactgtgatgtggtcatcggcatcgtgaacaacacagtttatgaccctctgcagcctgagctggacagcttcaaagaagagctggacaagtacttcaagaaccacacatctccagatgtggacctgggagacatctctggcatcaatgcctctgtggtgaacatccagaaggaaattgacaggctgaacgaagtggccaagaacctgaacgaaagcctcatcgacctgcaggagctgggcaagtacgagcagtacatcaagtggccttggtacatctggctgggcttcattgctggcctcatcgccatcgtgatggtgaccatcatgctgtgctgcatgaccagctgctgctcttgcctgaagggctgctgcagctgtggcagctgctgcaagtttgatgaagatgactctgagcctgtgctgaagggcgtgaagctgcactacaca

SEQ ID NO: 6 - protein, 1261 aa, Artificial Sequnce
  Chemically Synthesized, Spike OL
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITVAVACALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGANTSNQVTVLYQDVNCTEVPVAIHADQLTPTWRVYSTGSNVFKTRAGCLIGAEHVNNSYECDIPIGAGICASYQTQTNSPRRARSTASQSIIAYTMSLGAENSVAYSNNSIVIPTNFTISVTTEILPVSMTKTSVDCTMYICSDSTECSNPLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIRVTQNVLYENQKLIANQFNSAIGKIQDSLSSTASALGKLQDVVNQNAQALNTLVKQLSSTFSTISSVLNDILSRLDKVEAEVQIDRLITGRLQSLQTYVTQQLIRAAEIRASANLKATKMSECVLGQSKRVDFCGKGYHLMSFPQSAPHGVVFLHVTYVPAQEKNFTTAPATCHDGKAHFPREGVFVSNGTHWFVTQRNFDEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 7 - DNA, 3843 bp, Artificial Sequnce
  Chemically Synthesized, Spike OL with IgE leader
atggattggacctggattctttttctcgttgcagctgctacacgcgttcatagcagccagtgtgtgaacctgaccaccagaacacagctgcctcctgcctacaccaacagcttcaccagaggagtctactacccagacaaggtgttcagaagctctgtgctgcacagcacccaggacctcttcctgcctttcttcagcaacgtgacctggttccacgccatccacgtgtctggcaccaacggcaccaagagatttgacaaccctgtgctgcctttcaatgatggtgtgtactttgccagcacagagaagagcaacatcatccgaggctggatctttggcaccaccctggacagcaaaacacagagcctgctgatcgtgaataatgccaccaacgtggtcatcaaggtgtgtgagttccagttctgcaatgaccctttcctgggcgtgtactaccacaagaacaacaagtcctggatggagtctgagttccgagtgtacagctctgccaacaactgcacatttgaatatgtgtcccagcctttcctgatggacctggagggcaagcagggcaatttcaagaacctgagagaatttgtgttcaagaacatcgatggctacttcaagatctacagcaagcacacacccatcaacctggtgagagatcttcctcagggcttctctgccctggagcctctggtggacctgcccatcggcatcaacatcacccgctttcagaccctgctggccctgcacagaagctacctgaccccaggagacagcagcagcggctggacagctggagctgctgcctactacgtgggctacctgcagccaagaaccttcctgctgaagtacaacgaaaatggcaccatcactgtggctgtggcctgtgccctggaccctctttctgagaccaagtgcaccctgaagtccttcacagtggagaaaggcatctaccagaccagcaacttcagagttcagccaacagagagcatcgtgagatttccaaacatcaccaacctgtgtccttttggagaagtcttcaatgccaccagatttgcttctgtgtacgcctggaacagaaaaagaatcagcaactgtgtggctgactactctgtgctgtacaactctgcctccttctccaccttcaagtgctacggtgtgtctcctaccaagctgaatgacctgtgcttcaccaacgtgtatgctgacagctttgtcatcagaggagatgaagtgcggcagatcgcccctggccagacaggcaagattgctgactacaactacaagctgcctgatgacttcacaggctgtgtcatcgcctggaacagcaacaacctggacagcaaggtgggcggcaactacaactacctgtacagacttttcaggaagagcaacctgaagccttttgaaagagacatctccacagagatctaccaggctggcagcacaccctgcaatggagtggaaggcttcaactgctacttccctctgcagagctacggcttccagcccaccaatggcgtgggctaccagccttacagagtggtggtgctgtcctttgagctgctgcacgcccctgccacagtgtgtggccccaagaagagcaccaacctggtgaagaacaaatgtgtgaacttcaatttcaatggcctgacaggcacaggagtgctgacagagagcaacaagaagttcctgcctttccagcagtttggaagagacattgctgacaccacagatgctgtgagagatcctcagaccctggagatcctggacatcacaccctgctcctttggaggagtttctgtcatcacacctggagccaacaccagcaaccaagtgacagtgctgtaccaagatgtgaactgcacagaagttcctgtggccatccacgctgaccagctgaccccaacctggagagtctacagcacaggcagcaacgtgtttaaaacaagagctggctgcctgattggagcagagcacgtgaacaacagctatgaatgtgacatccctattggagctggcatctgtgccagctaccagacccaaaccaacagcccaagaagagccaggagcacagccagccagagcatcatcgcctacaccatgagcctgggagcagagaactctgtggcctacagcaacaacagcatcgtcatccccaccaacttcaccatctctgtgaccacagagatcctgcctgtgtccatgaccaagacatctgtggactgcaccatgtacatctgcagtgacagcacagaatgcagcaaccctctgctgcagtacggctccttctgcacccagctgaacagagccctgacaggcatcgctgtggagcaggacaagaacacacaggaagtgtttgcccaggtgaagcagatctacaaaacaccacccatcaaggactttggaggcttcaacttctcccagatcctgcctgaccccagcaagcccagcaagagaagcttcattgaagacctgctgttcaacaaagtgaccctggctgatgctggcttcatcaaacaatatggagactgcctgggagacattgctgccagagacctgatctgtgcccagaagtttaatggcctgactgtgctgcctcctctgctgacagatgaaatgatcgcccagtacacatctgccctgctggctggcaccatcacatctggctggacatttggagctggagctgccctgcagatcccttttgccatgcagatggcctacagatttaatggcatcagagtgacccagaacgtgctgtatgaaaaccagaagctgatcgccaaccagttcaactctgccatcggcaagatccaggacagcctgagcagcacagcctctgccctgggcaagctgcaggatgtggtgaaccaaaatgcccaggccctgaacaccctggtgaagcagctgagcagcaccttctccaccatctccagcgtgctgaatgacatcctgagccggctggacaaggtggaagctgaggtgcagatcgacagactcatcacaggccggctgcagagcctgcagacctacgtgacccagcagctgatcagagctgctgagatcagagcttctgccaacctgaaggccaccaagatgtcagaatgtgtgctgggccagagcaagagagtggacttctgtggcaaaggctaccacctgatgtccttccctcagtctgctcctcacggcgtggtgttcctgcacgtgacctacgtgcctgcccaggagaagaacttcaccacagctcctgccacctgccacgatggcaaagcccacttcccaagagaaggcgtctttgtgtccaatggcacccactggttcgtgacccagagaaactttgatgagcctcagatcatcaccacagacaacacatttgtttctggcaactgtgatgtggtcatcggcatcgtgaacaacacagtttatgaccctctgcagcctgagctggacagcttcaaagaagagctggacaagtacttcaagaaccacacatctccagatgtggacctgggagacatctctggcatcaatgcctctgtggtgaacatccagaaggaaattgacaggctgaacgaagtggccaagaacctgaacgaaagcctcatcgacctgcaggagctgggcaagtacgagcagtacatcaagtggccttggtacatctggctgggcttcattgctggcctcatcgccatcgtgatggtgaccatcatgctgtgctgcatgaccagctgctgctcttgcctgaagggctgctgcagctgtggcagctgctgcaagtttgatgaagatgactctgagcctgtgctgaagggcgtgaagctgcactacacataatga

SEQ ID NO: 8 - protein, 1279 aa, Artificial Sequnce
  Chemically Synthesized, Spike OL with IgE leader
MDWTWILFLVAAATRVHSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITVAVACALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGANTSNQVTVLYQDVNCTEVPVAIHADQLTPTWRVYSTGSNVFKTRAGCLIGAEHVNNSYECDIPIGAGICASYQTQTNSPRRARSTASQSIIAYTMSLGAENSVAYSNNSIVIPTNFTISVTTEILPVSMTKTSVDCTMYICSDSTECSNPLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIRVTQNVLYENQKLIANQFNSAIGKIQDSLSSTASALGKLQDVVNQNAQALNTLVKQLSSTFSTISSVLNDILSRLDKVEAEVQIDRLITGRLQSLQTYVTQQLIRAAEIRASANLKATKMSECVLGQSKRVDFCGKGYHLMSFPQSAPHGVVFLHVTYVPAQEKNFTTAPATCHDGKAHFPREGVFVSNGTHWFVTQRNFDEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 9 - protein, 18 aa, Artificial Sequnce
  Chemically Synthesized, IgE leader
MDWTWILFLVAAATRVHS

SEQ ID NO: 10 - protein, 9 aa, Artificial Sequnce
  Chemically Synthesized, H2-Kd epitope
PHGVVFLHV

SEQ ID NO: 11 - protein, 9 aa, Artificial Sequnce
  Chemically Synthesized, peptide 71663
VVFLHVTYV

SEQ ID NO: 12 - protein, 15 aa, Artificial Sequnce
  Chemically Synthesized, peptide 86
VVLSFELLHAPATVC

SEQ ID NO: 13 - protein, 15 aa, Artificial Sequnce
  Chemically Synthesized, peptide 90
KNKCVNFNFNGLTGT

SEQ ID NO: 14 - protein, 15 aa, Artificial Sequnce
  Chemically Synthesized, peptide 87
LLHAPATVCGPKKST

SEQ ID NO: 15 - protein, 15 aa, Artificial Sequnce
  Chemically Synthesized, peptide 176
SFPQSAPHGVVFLHV

SEQ ID NO: 16 - protein, 15 aa, Artificial Sequnce
  Chemically Synthesized, peptide 180
FTTAPAICHDGKAHF

SEQ ID NO: 17 - protein, 15 aa, Artificial Sequnce
  Chemically Synthesized, peptide 177
PHGVVFLHVTYVPAQ

SEQ ID NO: 18 - protein, 15 aa, Artificial Sequnce
  Chemically Synthesized, peptide 71
YNYKLPDDFTGCVIA

SEQ ID NO: 19 - protein, 15 aa, Artificial Sequnce
  Chemically Synthesized, peptide 75
VGGNYNYLYRLFRKS

SEQ ID NO: 20 - protein, 15 aa, Artificial Sequnce
  Chemically Synthesized, peptide 72
DDFTGCVIAWNSNNL